(19) United States
(12) Patent Application Publication
(10) Pub. No.: US 2004/0071787 A1
(43) Pub. Date: Apr. 15, 2004

(54) Pharmacologic, therapeutic and diagnostic regulation of FGF-1 export

(75) Inventors: Thomas Maciag, Freeport, ME (US); Brian Hampton, Germantown, MD (US); Wilson H. Burgess, Clifton, VA (US); Susan M. Gamble, Rockville, MD (US); Francesca Tarantini, Portland, ME (US); Anthony Q. Jackson, Landover, MD (US)

Correspondence Address: MORGAN, LEWIS & BOCKIUS LLP, 1701 MARKET STREET, PHILADELPHIA, PA, 19103-2921, US

(73) Assignee: Maine Medical Center Research Institute

(21) Appl. No.: 10/383,860

(22) Filed: Mar. 6, 2003

Related U.S. Application Data
(63) Continuation of application No. 09/137,713, filed on Aug. 21, 1998, now abandoned.
Application No. 09/137,713 is a continuation-in-part of application No. 08/848,911, filed on May 1, 1997, now abandoned.
Application No. 08/848,911 is a continuation-in-part of application No. 08/640,711, filed on May 1, 1996, now abandoned.

(51) Int. Cl. A61K 31/195; A61K 33/24

(52) U.S. Cl. 424/617; 514/566

(57) ABSTRACT

This invention relates to the regulation of FGF-1 export by cells. In addition, this invention provides methods of regulating the export of FGF-1 from the cell membrane, which methods include regulating the Syn-1 molecule to affect the formation of the FGF-Syn-1 complex, regulating the enzyme or protease which cleaves Syn-1 in the FGF-1 :Syn-1 complex to effect the release of FGF-1 from the cell membrane, or regulating the FGF-1:Syn-1 complex itself at or near the cell membrane. S100A13 is identified as an additional component of the FGF-1 export complex. Syn-1 truncation deletion mutants are also provided and functionally characterized. The invention also provides compositions related to the regulation of FGF-1 export.

### RELATED APPLICATIONS

This patent application is a continuation-in-part of U.S. application Ser. No. 08/848,911, filed May 1, 1997, which is a continuation-in-part of U.S. application Ser. No. 08/640,711 (abandoned), filed May 1, 1996, the entire contents of both of which are herein incorporated by reference.

### STATEMENT OF GOVERNMENT RIGHTS IN THE INVENTION

Part of the work performed during the development of this invention utilized U.S. Government funds under NIH grants HL44336 and HL32348. The U.S. Government may have certain rights in this invention.

### FIELD OF THE INVENTION

The present invention relates to the regulation of FGF-1 export.

### BACKGROUND OF THE INVENTION

Members of the fibroblast growth factor (FGF) family are potent regulators of developmental, physiological and pathophysiologic events in mammals. Therefore, it is important to understand the mechanism by which these factors regulate diverse biological processes such as mesoderm formation, atherogenesis, angiogenesis and neurogenesis.

The FGF family has been the subject of numerous comprehensive reviews. (See, e g., Burgess and Maciag, Annu. Rev. Biochem. 58: 575-606 (1989); Friesel and Maciag, FASEB J. 9: 919-925 (1995); Rifkin and Moscatelli, J. Cell Biol. 109: 1-6 (1989); Klagsbrun and Baird, Cell 67: 229-231 (1991).) The best characterized members of the FGF family are FGF-1 (acidic FGF) and FGF-2 (basic FGF). These two proteins are unusual growth factors in that they lack a classical signal sequence to direct their secretion through the conventional endoplasmic reticulum (ER)-Golgi apparatus. Ligation of a signal sequence to the FGF prototypes yields functional extracellular ligands with potent transforming potential in vitro and the ability to induce exaggerated angiogenic and neurotropic phenomena in vivo including the generation of atheroma-like lesions.

Because the mitogenic activities of FGF-1 and FGF-2 are mediated by a high affinity receptor at the plasma membrane surface, it has been proposed that a tightly regulated, yet unconventional, export pathway exists to regulate the export of these growth factors. Consequently, the regulation of human endothelial cell growth and the role of FGF-1 in mediating this process in vivo has been the subject of a number of studies. See, e.g., Maciag et al., Proc. Natl. Acad. Sci. USA 76: 5674-5678 (1979); Maciag et al., J. Cell Biol. 91: 420-426 (1981); Maciag et al., J. Cell Biol. 94: 511-520 (1982); Maciag et al., Science 225: 932-935 (1984).

The addition of a signal sequence (ss) to either FGF-1 or FGF-2 established the function of genes encoding these modified FGF proteins as transforming genes in a variety of target cells. See, e.g., Rogelj et al. Nature 331: 173-175 (1988); Blam et al., Oncogene. 3: 129-136 (1988); Jouanneau et al., Proc. Natl. Acad. Sci. USA 88: 2893-2897 (1991); Talarico and Basilico, Mol. Cell. Biol. 11:1138-1145 (1991); Forough et al., J. Biol. Chem. 268: 2960-2968 (1993). In vivo expression studies in the porcine iliac artery using a liposome:vector gene transfer method demonstrate an exaggerated hypertrophic response to the presence of extracellular FGF-1 including the formation of a prominent neointima containing numerous capillary and aorta-like structures (Nabel et al., Nature 362: 844-846 (1993)). In a similar manner, a transgene expressing a FGF-4₍ₛₛ₎:FGF-1 chimera under control of the α-crystallin promoter, which is active during lens development in the eye, induces a hypertrophic response that includes inappropriate formation of blood vessels and nerve bundles in the lens (Overbeek et al., Development (1994)).

In both studies, the transfer of the wild-type FGF-1 cDNA failed to yield pathologic consequences. Prior studies (e.g., Thompson et al., Science 241: 1349-1352 (1988)) have suggested that FGF-1 may have evolved without a functional signal sequence because expression would be accompanied by inappropriate export during certain situations, resulting in the formation of exaggerated vascular and neuronal structures. Thus, it appears that the pathway for FGF-1 export is tightly regulated in order to control the angiogenic and neurogenic activities of the protein.

In view of the detrimental effects that may ensue as a result of FGF-1 export, effective regulation is needed. The present invention exploits the discovery that FGF-1 is exported in response to temperature stress as a FGF-1 homodimer:synaptotagmin (Syn)-1 complex, or as a cofactor-mediated FGF-1:Syn-1 complex, and the existence of a Syn-1 cleavage enzyme or protease (Syn-1-CE) to regulate export and further provides other related advantages.

### SUMMARY OF THE INVENTION

The present invention generally provides methods of regulating FGF-1 export. In addition, the invention provides methods of regulating the formation of an FGF-1:Syn-1 complex or a complex comprising FGF-1:Syn-1, comprising inhibiting, blocking or binding FGF-1:Syn-1 or a complex comprising FGF-1:Syn-1, thereby regulating the export of FGF-1. It also provides methods comprising stimulating or enhancing the formation of an FGF-1:Syn1 complex or a complex comprising FGF-1:Syn-1, thereby regulating the export of FGF-1. Therapeutic agents useful in accordance with the aforementioned methods are also disclosed.

In one aspect, the present invention provides a method of regulating FGF-1 export from a cell by administering a therapeutic agent in an amount sufficient to inhibit the export of FGF-1, wherein the therapeutic agent inhibits the formation of a complex comprising FGF-1, and wherein the formation of this complex is a prerequisite to FGF-1 export. In one embodiment, the complex further comprises Syn-1. In another embodiment, the therapeutic agent inhibits the release of FGF-1 from a complex comprising FGF-1 and Syn-1. In another embodiment, the therapeutic agent inhibits the release of FGF-1 from, or the association of FGF-1 with, an intracellular vesicle. In another embodiment, the therapeutic agent may be an antibody, an immunologically active fragment of an antibody or an anti-inflammatory agent. In another embodiment, the therapeutic agent may be a Syn-1 antisense molecule or a Syn-1 antisense molecule encoding agent. In yet another embodiment, the therapeutic agent may be a ribozyme that specifically cleaves a Syn-1 encoding nucleic acid or a ribozyme encoding agent wherein the encoded ribozyme specifically cleaves a Syn-1 encoding nucleic acid.

It is another aspect of the invention to provide a method of regulating FGF-1 export from a cell by administering a therapeutic agent in an amount sufficient to promote the export of FGF-1, wherein the therapeutic agent promotes the formation of a complex comprising FGF-1, and wherein the formation of the complex is a prerequisite for FGF-1 export. In one embodiment, the complex further comprises Syn-1 and in another embodiment the therapeutic agent promotes the release of FGF-1 from Syn-1 at or near the plasma membrane. According to another embodiment, the therapeutic agent promotes the release of a complex comprising FGF-1 from an intracellular vesicle. In yet another embodiment, the therapeutic agent promotes the association of a complex comprising FGF-1 with an intracellular vesicle.

Turning to another aspect, the invention provides a method of regulating FGF-1 export, comprising inhibiting a protease that cleaves Syn-1 in an FGF-1:Syn-1 complex, thereby inhibiting the export of the FGF-1:Syn-1 complex.

Another aspect of the invention provides a method of regulating FGF-1 export, comprising increasing the activity of a protease that cleaves Syn-1 in an FGF-1:Syn-1 complex, thereby promoting the release of FGF-1 at or near the plasma membrane.

In another aspect, the invention also provides a therapeutic agent which regulates the export of FGF-1 from a cell, wherein the agent inhibits export of FGF-1, does not inhibit secretion of a leader sequence-containing protein, and does inhibit the binding between the FGF-1 and an intracellular vesicle. In certain embodiments, the agent inhibits the formation of an FGF-1:Syn-1 complex. In certain other embodiments, the agent inhibits the release of FGF-1 from a complex comprising FGF-1. In still other embodiments, the agent inhibits the association of FGF-1 with an intracellular vesicle, while in other embodiments the agent inhibits the release of FGF-1 from an intracellular vesicle. In some embodiments, the therapeutic agent may be an antibody, an immunologically active fragment of an antibody or an anti-inflammatory agent. In some other embodiments the therapeutic agent may be a Syn-1 antisense molecule or a Syn-1 antisense molecule encoding agent. In certain other embodiments the therapeutic agent may be a ribozyme that specifically cleaves a Syn-1 encoding nucleic acid; or a ribozyme encoding agent, wherein the ribozyme specifically cleaves a Syn-1 encoding nucleic acid. In still other embodiments the invention provides a therapeutic agent that is a protein derived from a recombinant molecule.

It is another aspect of the invention to provide an isolated, purified protease that cleaves Syn-1 in an FGF-1:Syn-1 complex and which thereby regulates the export of FGF-1. In another aspect, the invention provides an FGF-1 homodimer:Syn-1 complex.

Turning to another aspect, the invention provides a nucleic acid molecule encoding a truncated Syn-1 molecule, comprising a truncated nucleotide sequence of SEQ ID NO:1. In certain embodiments, the truncated Syn-1 molecule lacks a site for cleavage by a protease that cleaves Syn-1 in an FGF:Syn-1 complex. In certain other embodiments, the truncated Syn-1 molecule lacks a membrane binding site, and in certain further embodiments the membrane binding site binds to the plasma membrane. In certain further embodiments the membrane binding site binds to a cytoplasmically accessible component of a plasma membrane. In certain embodiments the truncated Syn-1 molecule lacks a membrane binding site that is a phosphatidylserine binding site.

In another aspect, the invention provides a truncated Syn-1 molecule, comprising a truncated amino acid sequence of SEQ ID NO:2. In one embodiment, the truncated Syn-1 molecule lacks a site for cleavage by a protease that cleaves Syn-1 in an FGF:Syn-1 complex. In another embodiment, the truncated Syn-1 molecule lacks a membrane binding site. In a further embodiment, the membrane binding site binds to the plasma membrane. In a further embodiment, the membrane binding site binds to a cytoplasmically accessible component of a plasma membrane. In a further embodiment, the membrane binding site comprises a phosphatidylserine molecule.

In addition, it is an aspect of the present invention to provide a nucleic acid molecule encoding a truncated Syn-1 molecule, comprising a nucleotide sequence of SEQ ID NO:1 having a deletion such that the truncated Syn-1 molecule lacks at least one of a lipid binding domain, a domain containing a site for cleavage by a protease that cleaves Syn-1 in an FGF:Syn-1 complex, a calcium binding domain or a domain containing a casein kinase II phosphorylation site. Another aspect of the invention is to provide a truncated Syn-1 molecule, comprising an amino acid sequence of SEQ ID NO:2 having a deletion such that the truncated Syn-1 molecule lacks at least one of a lipid binding domain, a domain containing a site for cleavage by a protease that cleaves Syn-1 in an FGF:Syn-1 complex, a calcium binding domain or a domain containing a casein kinase II phosphorylation site.

In another aspect, the invention provides a nucleic acid molecule encoding a truncated Syn-1 molecule, comprising a nucleic acid sequence of SEQ ID NO:3 or portion thereof. It is another aspect of the invention to provide a truncated Syn-1 molecule, comprising an amino acid sequence of SEQ ID NO:4 or portion thereof. Yet another aspect of the invention provides a nucleic acid molecule encoding a truncated Syn-1 molecule, comprising a nucleic acid sequence of SEQ ID NO:5 or portion thereof, and in another aspect the invention provides a truncated Syn-1 molecule, comprising an amino acid sequence of SEQ ID NO:6 or portion thereof.

Turning to another aspect, the invention provides a nucleic acid molecule encoding a Syn-1 molecule that lacks a site for cleavage by a protease that cleaves Syn-1 in an FGF:Syn-1 complex.

It is another aspect of the invention to provide a method of regulating FGF-1 export from a cell comprising administering a therapeutic agent in an amount sufficient to inhibit the export of FGF-1, wherein the therapeutic agent inhibits the binding of a complex comprising FGF-1 to a cellular membrane, and wherein the formation of the complex is a prerequisite to FGF-1 export. In certain embodiments, the cellular membrane is a plasma membrane or a cytoplasmically accessible component thereof. In certain embodiments the cytoplasmically accessible component comprises a phosphatidylserine molecule. In another embodiment the cellular membrane is an intracellular vesicle membrane or a cytoplasmically accessible component thereof, and in some embodiments the cytoplasmically accessible component comprises a phosphatidylserine molecule. In other embodiments of this aspect of the invention, the complex further comprises Syn-1.

It is also an aspect of the invention to provide a method of regulating FGF-1 export from a cell comprising administering a therapeutic agent in an amount sufficient to inhibit the export of FGF-1, wherein the therapeutic agent inhibits the release of a complex comprising FGF-1 from a cellular membrane, wherein the formation of the complex is a prerequisite to FGF-1 export. In one embodiment the cellular membrane is a plasma membrane or a cytoplasmically accessible component thereof, and in certain further embodiments the cytoplasmically accessible component comprises a phosphatidylserine molecule. In another embodiment, the cellular membrane is an intracellular vesicle membrane or a cytoplasmically accessible component thereof and in certain further embodiments the cytoplasmically accessible component comprises a phosphatidylserine molecule. In another embodiment, the complex further comprises Syn-1.

Turning to another aspect, the invention provides a method of regulating FGF-1 export from a cell comprising administering a therapeutic agent in an amount sufficient to promote the export of FGF-1, wherein the therapeutic agent promotes the binding of a complex comprising FGF-1 to a cellular membrane, wherein the formation of the complex is a prerequisite to FGF-1 export. In certain embodiments the cellular membrane is a plasma membrane or a cytoplasmically accessible component thereof, and in certain further embodiments the cytoplasmically accessible component comprises a phosphatidylserine molecule. In other embodiments the cellular membrane is an intracellular vesicle or a cytoplasmically accessible component thereof, and in certain further embodiments the cytoplasmically accessible component comprises a phosphatidylserine molecule. In another embodiment, the complex further comprises Syn-1.

In another aspect the invention provides a method of regulating FGF-1 export from a cell comprising administering a therapeutic agent in an amount sufficient to promote the export of FGF-1, wherein the therapeutic agent promotes the release of a complex comprising FGF-1 from a cellular membrane, wherein the formation of the complex is a prerequisite to FGF-1 export. In certain embodiments, the cellular membrane is a plasma membrane or a cytoplasmically accessible component thereof, and in certain further embodiments the cytoplasmically accessible component comprises a phosphatidylserine molecule. In other embodiments, the cellular membrane is an intracellular vesicle or a cytoplasmically accessible component thereof, and in certain further embodiments the cytoplasmically accessible component comprises a phosphatidylserine molecule. In another embodiment, the complex further comprises Syn-1.

In other embodiments of the several aspects of the invention just described that are methods of regulating FGF-1 export from a cell, the complex further comprises S100A 13. In further embodiments, the therapeutic agent binds to S100A13, and in certain further embodiments, the therapeutic agent is an amlexanox compound. In certain embodiments, the amlexanox compound is amlexanox AA673, amlexanox derivative AA617 or amlexanox derivative AA648.

Turning to another aspect, the invention provides a method of identifying a component in a complex the formation of which is a prerequisite to FGF-1 export, comprising isolating from a cell capable of expressing and exporting FGF-1 a complex comprising FGF-1; and determining the presence in the isolated complex of one or more molecular species, and therefrom identifying a component of the complex.

In still another aspect, the invention provides a method of identifying a component in a complex, the formation of which is a prerequisite to FGF-1 export, comprising isolating from a cell capable of expressing and exporting FGF-1 a complex comprising FGF-1, determining whether formation of the isolated complex is a prerequisite to FGF-1 export, and detecting the presence, in isolated complexes the formation of which is determined to be a prerequisite to FGF-1 export, of one or more molecular species, and therefrom identifying a component of the complex.

In still another aspect, the invention provides a method of identifying a component in a complex the formation of which is a prerequisite to FGF-1 export, comprising isolating from a cell capable of expressing and exporting FGF-1 a complex comprising FGF-1 and Syn-1, and detecting the presence in the isolated complex of one or more molecular species, and therefrom identifying a component of said complex. In certain embodiments of this aspect of the invention, Syn-1 is a truncated Syn-1. In other embodiments of the several aspects of the invention just described that are methods of identifying a component in a complex the formation of which is a prerequisite to FGF-1 export, the molecular species preferentially associates with FGF-1, and in other embodiments the molecular species associates indirectly with FGF-1. In one embodiment, isolation of the complex comprising FGF-1 follows export of the complex and in another embodiment isolation of the complex comprising FGF-1 precedes export of the complex. In another embodiment the complex comprising FGF-1 further comprises dimeric FGF-1. In another embodiment, the complex comprising FGF-1 is isolated from a source that is cell conditioned medium, cultured cells or a biological tissue, which biological tissue is, according to a further embodiment, brain. In another embodiment of the aspect of the invention that is a method of identifying a component in a complex the formation of which is a prerequisite to FGF-1 export, the complex comprising FGF-1 further comprises Syn-1. In another embodiment, the complex comprising FGF-1 further comprises S100A13. In another embodiment, FGF-1 in an isolated complex is a product of an endogenous gene, while in yet another embodiment FGF-1 in an isolated complex is a product of a transfected gene. In certain embodiments, the transfected gene encodes full length FGF-1, and in certain other embodiments the transfected gene encodes truncated FGF-1. In certain further embodiments, the transfected gene encodes FGF-1₍₂₁₋₁₅₄₎. In another embodiment, the transfected gene encodes FGF-1 having an N-terminal deletion of at least 14 amino acids.

In other embodiments of the aspect of the invention directed to a method of identifying a component in a complex, the formation of which is a prerequisite to FGF-1 export, as described above, the complex comprising FGF-1 is isolated by an affinity technique. In certain embodiments the affinity technique is an immunological technique and in certain further embodiments the immunological technique is immunoaffinity chromatography, immunoprecipitation or solid phase immunoadsorption. In certain other embodiments the affinity technique is heparin binding.

Turning to yet another aspect of the invention, a method is provided of identifying an agent that inhibits formation of a complex comprising FGF-1 wherein the formation of the complex is a prerequisite to FGF-1 export, comprising exposing to a candidate agent a cell capable of expressing FGF-1 and of forming a complex which is a prerequisite to exporting FGF-1, the complex including FGF-1; and determining the presence or absence of the complex, and therefrom identifying an agent that inhibits the formation of the complex that is a prerequisite to FGF-1 export.

In still another aspect, the invention provides a method of identifying an agent that promotes formation of a complex comprising FGF-1 wherein the formation of the complex is a prerequisite to FGF-1 export, comprising exposing to a candidate agent a cell capable of expressing FGF-1 and of forming a complex which is a prerequisite to expressing and exporting FGF-1, the complex including FGF-1; and determining the presence or absence of the complex, and therefrom identifying an agent that promotes the formation of the complex that is a prerequisite to FGF-1 export. In certain embodiments of either of these aspects of the invention that provide a method of identifying an agent that inhibits formation of a complex comprising FGF-1 and wherein the formation of the complex is a prerequisite to FGF-1 export, the step of determining the presence or absence of the complex comprises determining the presence or absence of at least one molecular species that preferentially associates with FGF-1. In certain other embodiments of either of these aspects of the invention, the step of determining the presence or absence of the complex comprises determining the presence or absence of at least one molecular species that associates indirectly with FGF-1.

It is another aspect of the invention to provide a nucleic acid molecule encoding a truncated FGF-1, comprising a nucleotide sequence of SEQ ID NO:7 having a deletion. In one embodiment, the truncated FGF-1 binds Syn-1. In another embodiment, the truncated FGF-1 binds phosphatidylserine.

Another aspect of the invention provides a truncated FGF-1 molecule, comprising an amino acid sequence of SEQ ID NO:8 having a deletion. In one embodiment, the truncated FGF-1 binds Syn-1 and in another embodiment the truncated FGF-1 binds phosphatidylserine.

It is yet another aspect of the invention to provide a nucleic acid molecule encoding a fusion protein comprising a nucleic acid sequence encoding a truncated FGF-1 that binds Syn-1 and a nucleic acid sequence encoding a desired polypeptide. In still another aspect, the invention provides a nucleic acid molecule encoding a fusion protein comprising a nucleic acid sequence encoding a truncated FGF-1 that binds phosphatidylserine and a nucleic acid sequence encoding a desired polypeptide.

These and other aspects of the present invention will become evident upon reference to the following detailed description and attached drawings. In addition, various references are set forth herein which describe in more detail certain aspects of this invention, and are therefore incorporated by reference in their entireties.

### BRIEF DESCRIPTION OF THE FIGURES

FIG. 1 illustrates a working model of the FGF-1 export pathway as a diagrammatic flow chart of the events involved.

FIG. 2 is an immunoblot showing how a Syn-1 fragment is released in response to temperature stress. The immunoblot analysis shown used an antibody to Syn-1 on medium conditioned by heat shock (2 hr, 42° C.; lane 2) as compared with media that was not heat-shock conditioned (2 hr, 37° C.; lane 1) from FGF-1 NIH 3T3 transfectants, activated by addition of (NH₄)₂S0₄ and absorbed/eluted from heparin Sepharose™. Recombinant p40 Syn-1 (100 ng) is shown in lane 3 (positive control); it has an apparent Mᵣ of about 42 kDa as shown in the immunoblot. In lane 2, a Syn-1 fragment—i.e., a p40 heparin-binding protein—having an apparent Mᵣ of about 40 kDa is shown.

FIG. 3 is a graph showing the binding of phosphatidylserine (pS) by recombinant p40 Syn-1. Recombinant human p40 Syn-1 was expressed in the pET3 expression system, purified by heparin affinity and RP-HPLC, iodinated using lactoperoxidase methods, and added to plastic wells coated with phospholipids as described in Perin et al., Nature 345: 260-3 (1990). Across the horizontal axis, the following abbreviations appear: Negative control, no phospholipid; pS, phosphotidylserine; pC, pl choline; pE, pL ethanol-amine; pI, pL inositol; and pG, pL glycerol. Results were reported as CPMs bound (±std deviation) after three washes with 0.1M Tris, pH 7.4, containing 0.15M NaCl ; cpm's are shown on the vertical axis.

FIGS. 4A and 4B depict the release of FGF-1 as high Mᵣ complexes in response to temperature stress as shown by an immunoblot analysis of conditioned medium from heat shocked FGF-1 NIH 3T3 transfectants, activated by addition of either 0.1% DTT (FIG. 4A) or 90% (sat.) ammonium sulfate ((NH₄)₂S0₄) (FIG. 4B) adsorbed to heparin-SEPHAROSE™, gradient eluted with NaCl. A non-reduced, limited SDS-PAGE FGF-1 immunoblot was performed using antiserum raised to FGF-1 on the samples as described by Jackson et al., PNAS 89: 10691-5 (1992). Mᵣs were assigned for comparative purposes and appear on the vertical axis. All samples shown are over heparin-SEPHAROSE™.

FIGS. 5A and 5B show the results of native PAGE gel shift analysis of FGF-1 binding to phospholipids (pL). Native PAGE (pH 6.8) analysis was performed with varying amounts of phosphatidylserine (pS) in FIG. 5A and phosphatidylethanolamine (pEA) in FIG. 5B, but with a constant amount of [¹²⁵I]-FGF-1. Amounts of pS and pEA are expressed in nM increments across the top of each Figure. The pL were premixed with cold FGF-1, followed by addition of [¹²⁵I]-FGF-1 probe. The entire sample was subjected to electrophoresis, except the last lane in each gel. The last lane contained either pS or pEA containing cold FGF-1, loaded without probe; [¹²⁵I]-FGF-1 was added to the top of the lane immediately prior to electrophoresis.

FIGS. 6A and 6B are immunoblots showing Cu²⁺ induction of FGF-1 and Syn1 heterodimers. In FIG. 6A, purified FGF-1 and p40 Syn-1 were incubated with and without Cu²⁺ and were then resolved by non-reduced SDS-PAGE. In FIG. 6B, procedures were identical to those used in 6A, except immunoblotting was performed with FGF-1 antibody. In FIGS. 6A and 6B, the results using FGF-1, FGF-1+p40 Syn, and p40 Syn (with and without Cu²⁺) are indicated across the horizontal axes; Mᵣ are shown on the vertical axes. Use of the designation “p40” herein refers to p40 Syn-1, which has also been previously referred to as “p45” or “p40/45”.

FIGS. 7A and 7B illustrate the resolution of FGF-1 and Syn-1 as a low-affinity heparin affinity high Mᵣ complex using non-reduced limited SDS-PAGE FGF-1/Syn-1 immunoblot analysis. The immunoblots shown in FIGS. 7A and B were prepared as described in FIG. 4. The immunoblot shown in FIG. 7A was probed with anti-FGF-1; the immunoblot shown in FIG. 7B was probed with anti-Syn-1. All samples shown are over heparin-SEPHAROSE™. In both FIGS. 7A and 7B, NaCl concentration (M) is shown on the horizontal axes and Mᵣ on the vertical axes. Reference samples of FGF-1-alpha (FIG. 7A) and Syn (FIG. 7B) are also indicated.

FIG. 8 shows that antisense (γ) Syn-1 represses the release of FGF-1 in response to heat shock. Immunoblot analysis of conditioned medium from heat shocked FGF-1 NIH 3T3 transfectants cotransfected with antisense (γ) Syn-1 cDNA, as compared with control conditioned media; and cell lysates under comparable conditions. pXZ38 cells=control transfectants; #10 Syn-1 and #19 Syn-1 are low and high FGF-1 expressing γ-Syn-1, FGF-1 NIH 3T3 cell co-transfectants, respectively.

FIGS. 9A and 9B illustrate that antisense (γ) Syn-1 represses the growth of NIH 3T3 cells but does not attenuate the ability of exogenous FGF-1 to stimulate growth. FIG. 9A shows the results of a growth assay in which pXZ28 (FGF-1 transfected NIH 3T3 cells) are compared to pXZ38-Syn-1 antisense transfected cells. FIG. 9B shows results of a growth assay in which Syn-1 antisense transfected cells in response to FGF-1. The solid (closed) bars represent the addition of FGF-1 in FIG. 9B. Graphs representing the fetal bovine serum (FBS) growth response curves of FGF-1 and γ-Syn-1, FGF-1 co-transfectants as a function of FBS (FIG. 9A), and the growth response of γ-Syn-1, FGF-1 co-transfectants (FIG. 9B) as a function of FBS in the presence and absence of FGF-1 are shown. In both FIGS. 9A and 9B, serum (%, v/v) is illustrated on the horizontal axes, while cells/well (×10⁻⁴ and 10⁻⁵, respectively) are plotted on the vertical axes.

FIGS. 10A and 10B show that organelle restriction of intracellular FGF-1 attenuates the FGF-1 export pathway which is able to recognize cytosolic FGF-1 as a high Mᵣ β-gal chimera. 3T3 cell transfectants expressing FGF-1 either as a COOH-terminal β-gal chimera or as an NH₂-terminal SV4OT NLS COOH-terminal β-gal chimera were examined for the cytosolic and nuclear presence of the reporter gene using immunofluorescence microscopy with either an anti-β-gal antibody or X-gal staining (FIG. 10A). Total cell lysate processed for β-gal immunoblot analysis is shown at FIG. 10B.

FIG. 11 shows the results of putative FGF-1:β-Gal chimera associated proteins resolved by β-gal immunoprecipitation of metabolically labeled FGF-1:β-Gal NIH 3T3 cell transfectants. Cells were radiolabeled with (³⁵S)-met/cys, heat shocked (at 42° C., for 2 hr.), conditioned media was collected, cells were lysed, immunoprecipitated with anti-β-gal antibody and resolved by SDS-PAGE. Arrows mark the co-precipitated bands of interest. Times, temperature conditions, and media vs. lysates are indicated on the horizontal axis, while Mᵣ are indicated on the vertical axis.

FIG. 12 shows FGF-1 mutants and chimeric constructs prepared by recombinant circle PCR (RC-PCR; see Friedman, et al., Biochem. Biophys. Res. Commun. 198: 1203-8 (1994)). Stable 3T3 cell transfectants for each of these constructs have been isolated, and the cytosolic level of the individual FGF-I₂₁₋₁₅₄ mutant/chimeric proteins are similar as measured by FGF-1 immunoblot analysis. The traffic of the individual constructs are shown; these data are derived from cell fractionation/immunoblot analysis, X-gal staining and β-gal immunohistochemical analysis as described in FIG. 10. Export (indicated as “secretion”) of the individual construct was measured as described in FIG. 10. NTS =Nuclear Translocation Signal.

FIGS. 13A and 13B show the results of an FGF-1/Syn-1 immunoprecipitation/Syn-1 immunoblot analysis of ovine brain samples resolved by low heparin-™ affinity and Sephadex G-100 gel exclusion chromatography. Western blots of samples were obtained from a Sephadex™ G-100 column. The gel in FIG. 13A shows FGF-1 immunoprecipitation followed by Syn-1 immunoblot analysis. The gel in FIG. 13B shows Syn-1 immunoprecipitation followed by Syn-1 immunoblot analysis.

FIG. 14 illustrates a compilation of many Syn-1 and FGF-1 immunoblot and RP-HPLC analyses of the FGF-1:Syn-1 complex. The top panel represents RP-HPLC resolution, following immunoblot analysis of a heparin affinity chromatography identified fraction of ovine brain extract containing both FGF-1 and Syn-1. The middle panel shows a single peak representing the RP-HPLC resolution of the stable, pure protein complex comprising FGF-1 and Syn-1. The bottom panel, showing at least six (6) significant protein peaks, represents RP-HPLC resolution of the FGF-1:Syn-1 complex following chaotropic denaturation with 8M guanidinium HCl. The multiple peaks shown in the lower panel represent proteins having retention times that are different from that of either the FGF-1 or Syn-1 components of the protein complex.

FIG. 15 illustrates a deletion analysis of p40 Syn-1. The domain structure for p65 Syn-1 is shown, and the domains include: the intravesicular domain, transmembrane domain and the extravesicular domain (facing cytosol) comprising the multimerization (M) domain, Ca²⁺-pS-binding (pS) domain, clatharin-binding (C) domain and the neurexin (N) domain. Individual domain deletion (Δ) Syn-1 mutants are shown as β-gal chimeric structures.

FIG. 16 depicts the release of the extravesicular domain of Syn-1 from p40 Syn-1 and FGF-1:β-gal NIH 3T3 cell co-transfectants. Panels A and C are immunoblots developed with antibodies specific for Syn-1, and panels B and D are immunoblots developed with antibodies specific for FGF-1. Panel A: Lanes 1 and 2 are the lysates from p40 Syn-1 and FGF-1:β-gal NIH 3T3 cell co-transfectants at 37° C. untreated or treated with brefeldin A, respectively. Lane 3 is 50 ng of recombinant rat Syn-1 extravesicular domain. Lanes 4 and 5 are conditioned medium collected from untreated cells at 37° C. and 42° C., respectively. Lanes 6 and 7 are conditioned medium collected from cells treated with brefeldin A at 37 and 42° C., respectively. Arrow indicates p40 Syn-1. Panel B: Lanes 1 and 2 are the lysates from p40 Syn-1 and FGF-1:β-gal NIH 3T3 cell transfectants at 37° C. untreated or treated with brefeldin A, respectively. Lanes 3 and 4 are conditioned medium collected from untreated cells at 37 and 42° C., respectively. Lanes 5 and 6 are conditioned medium collected from cells treated with brefeldin A at 37 and 42° C., respectively. Arrow indicates FGF-1:βgal. Panel C: Lanes 1 and 2 are the lysates from p40 Syn-1 and FGF-1:β-gal NIH 3T3 cell transfectants at 37° C. untreated or treated with 2-dOG, respectively. Lane 3 is 50 ng of recombinant rat Syn-1 extravesicular domain. Lanes 4 and 5 are conditioned medium collected from untreated cells at 37 and 42° C., respectively. Lanes 6 and 7 are conditioned medium collected from cells treated with 2-dOG at 37 and 42° C., respectively. Arrow indicates p40 Syn-1. Panel D: Lanes 1 and 2 are the lysates from p40 Syn-1 and FGF1:β-gal NIH 3T3 cell transfectants at 37° C. untreated or treated with 2-dOG, respectively. Lanes 3 and 4 are conditioned medium collected from untreateed cells at 37 and 42° C., respectively. Lanes 5 and 6 are conditioned medium collected from cells treated with 2-dOG at 37 and 42° C., respectively. Arrow indicates FGF-1:β-gal.

FIG. 17 depicts amlexanox inhibition of FGF-1:β-gal and Syn-1 release from NIH 3T3 and FGF-1:β-gal:p65 Syn-1 co-transfectants.

FIG. 18 depicts effects of amlexanox on NIH 3T3 FGF-1 :β-gal:p40 Syn-1 co-transfectants.

FIG. 19 depicts effects of amlexanox and amlexanox derivatives on NIH 3T3 FGF-1 transfectants.

### DETAILED DESCRIPTION OF THE INVENTION

Definitions

An understanding of the present invention may be aided by reference to the following definitions and explanation of conventions used herein. In accordance with the present invention and as used herein, the following terms are defined to have following meanings, unless explicitly stated otherwise:

As used herein, “export” of a protein refers to a metabolically active process of transporting a translated cellular product to the extracellular spaces or to the cell membrane by a mechanism other than by a leader sequence.

As used herein, “purified” means that the desired purified protein or fusion protein produces a single band on a conventional assay using an appropriate label, e.g., the protein stain, Coomassie blue.

As used in the present context, although the hallmark of “stress” in the cell is the “heat shock response,” the term includes a broad variety of stressors which may induce the response; e.g., amino acid analogs, puromycin, ethanol, heavy metal ions, arsenicals, tissue explantation, infection, etc. The reference to “heat shock” or “temperature stress,” therefore, is meant to include any stress that results in the export of FGF-1 or the formation of the FGF-1:Syn-1 complex.

As used herein, amlexanox has its standard meaning in the art, and refers to the compound of the formula (Ia)

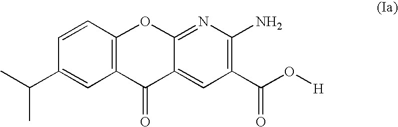

As used herein, an “amlexanox compound” includes amlexanox itself as well as derivatives and analogs thereof, including the salts (acid and base addition salts), solvates, isolated enantiomers, isolated diastereomers, and isolated tautomers of amlexanox or a derivative or analog thereof. The term also includes mixtures of any the proceeding compounds.

In general, an amlexanox compound as used in the methods of the present invention has the formula (I)

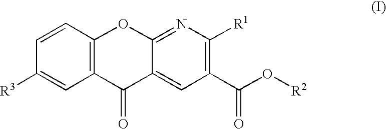

wherein each of R¹, R² and R³ are organic or inorganic moieties, including halogen and hydrogen. Each of R¹, R² and R³ preferably has less than 100 atoms, more preferably less than 50 atoms, and still more preferably less than 25 atoms. Preferred organic and inorganic moieties are formed from one or more of bromine, carbon, chlorine, fluorine, hydrogen, iodine, nitrogen, oxygen, phosphorus and sulfur. R³ preferably contains at least 2 carbon atoms.

Exemplary compounds of formula (I) have, independently at each location or occurrence,

R¹ and R³ selected from amino, halogen, hydrazino, hydrocarbyl, hydrocarbylcarbonyloxy, hydrocarbylcarbonylsulfide, hydrocarbyloxy, hydrocarbylsulfide, hydrogen, hydroxyl, nitro, oxime, oxo, substituted amino, substituted hydrocarbyl, thiol, and groups of the formulae: —S(O)ₙH, —S(O)ₙL, —S(O)ₘOH, —S(O)ₘOL, —OS(O)ₘOL, and —O(S)ₘOH, wherein L is a hydrocarbyl group, m is independently 1 or 2, and n is independently 0, 1 or 2;

with the proviso that R³ contains at least 2 carbon atoms; and

R² selected from hydrogen, hydrocarbyl and substituted hydrocarbyl.

In a preferred embodiment, the amlexanox compound of formula (I) has

R¹ selected from C₁-C₅alkoxy; amino, di(C₁-C₅alkyl)amino, di(C₁-C₅alkoxy)-amino; C₁-C₅carboxylic ester; carboxylic acid or salt thereof; halogen, C₁-C₆hydrocarbyl; C₁-C₆hydrocarbyl substituted with one or more of carboxylic acid, and C₁-C₅carboxylic ester; hydrogen, mono(C₁-C₅alkyl)amino, and mono(C₁-C₅alkoxy)amino;

R² selected from hydrogen and hydrocarbyl; and

R³ selected from hydrocarbyl of at least 2 carbon atoms, and substituted hydrocarbyl where the hydrocarbyl group has at least 2 carbon atoms.

In a more preferred embodiment, the amlexanox compound of formula (I) has

R¹ selected from amino and C₁-C₅alkyl;

R² selected from hydrogen and C₁-C₅alkyl; and

R³ selected from C₂-C₅alkyl.

The amlexanox compounds useful in the present invention may contain two or more asymmetric carbon atoms and thus exist as enantiomers and diastereomers. Unless otherwise noted, the amlexanox compounds include all enantiomeric and diastereomeric forms of the compounds. Pure stereoisomers, mixtures of enantiomers and/or diastereomers, and mixtures of different amlexanox compounds may be used in the methods of the present invention. Thus, the amlexanox compounds may be used in the form of racemates, racemic mixtures and as individual diastereomers, or enantiomers. A racemate or racemic mixture does not imply only a 50:50 mixture of stereoisomers. The compounds of formula (I) may also exist in tautomeric forms and the invention allows for the use of both mixtures and separate individual tautomers.

The phrase “independently at each location or occurrence” is intended to mean (i) when any variable occurs more than one time in a compound, the definition of that variable at each occurrence is independent of its definition at every other occurrence; and (ii) the identity of any one of two different variables is selected without regard the identity of the other member of the set. However, combinations of substituents and/or variables are permissible only if such combinations result in stable compounds.

“Acid addition salts” refers to those salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Base addition salts” refers to those salts which retain the biological effectiveness and properties of the free aicds and which are not biologically or otherwise undesirable, formed with inorganic bases such as sodium hydroxide and potassium hydoxide, or organic bases such as amines.

“Hydrocarbyl” refers to a mono- or poly-valent radical formed entirely from carbon and hydrogen. Exemplary hydrocarbyl groups include, without limitation, alkyl, alkenyl, alkynyl, aryl, aralkyl, alkylaryl, alkenyl-substituted aryl, aryl-substituted alkenyl, alkynyl-substituted aryl, aryl-substituted alkynyl, biaryl, cycloalkyl, cycloalkenyl, bicycloalkyl, bicycloalkenyl, alkylcycloalkyl, alkenylcycloalkyl, alkynylcycloalkyl, aryl-substituted cycloalkyl, cycloalkyl-substituted aryl, aryl-substituted cycloalkenyl, cycloalkenyl-substituted aryl, and aryl-fused cycloalkyl.

“Hydrazino” refers to a group having the formula —N═N-(hydrocarbyl or substituted hydrocarbyl).

“Oxime” refers to a group having the formula —N═ (hydrocarbyl or substituted hydrocarbyl).

“Oxo” refers to a carbonyl group, such that when a substituent, e.g., R¹, is oxo, then R¹ represents the oxygen atom of a carbonyl group, where the carbon atom of the carbonyl group is the carbon atom to which R¹ is identified as being attached.

“Substituted Amino” refers to an amino group wherein one or both of the amino hydrogen atoms are replaced with a non-hydrogen atom. The non-hydrogen atom may be, for example, a carbon or a non-carbon atom, where oxygen is an exemplary non-carbon atom. The carbon atom may form part of a hydrocarbyl or substituted hydrocarbyl group as defined herein. The oxygen or other non-carbon atom may be bonded to hydrogen, a hydrocarbyl or substituted hydrocarbyl group. Substituted amino moieties include, without limitation, hydrocarbylamino, dihydrocarbylamino, acylamino, acylhydrocarbylamino, hydrocarbyloxyamino,

“Substituted Hydrocarbyl” refers to a hydrocarbyl moiety having one or more (including all) hydrogens thereof replaced with an atom other than carbon or hydrogen. For example, the substituted hydrocarbyl may contain a single carbon atom, as in cyano (—CN), carboxy (—COOH), hydrocarbyl-oxycarbonyl (i.e., carboxylate esters, —COOR) and aminocarbonyl (i.e., amido, CO—NH,). The atom other than carbon or hydrogen as referred to above may be bonded to a carbon of the hydrocarbyl group by any of a single, double or triple bond. In addition, the atom other than carbon or hydrogen as referred to above may, simultaneously, be bonded to more than one carbon atom of the hydrocarbyl moiety, so as to form, for example, a heterocyclic structure. As further examples, one or more hydrogen atoms of the hydrocarbyl group may be substituted with an equal number of halogen atoms. In addition, substituted hydrocarbyl specifically includes —C(═N—OH)(C(═O)Ohydrocarbyl); —CHCl—C(═O)—O(hydrocarbyl); and —C(Me₂)—C(═O)—O(hydrocarbyl).

Amlexanox compounds, as that term is used herein, may be prepared according to procedures set forth in the following publications: Ibrahim et al., Indian J. Heterocycl. Chem., 4(1):15-18, 1994; Isoda et al., JP 03086884 A2 (91.04.11); Kubo et al., Chem. Pharm. Bull. 34(3):1108-17, 1986; Hayashi, N. and Imanishi M., J. Labeled Compd. Radiopharm. 23(3):339-41, 1986; Nohara, A., EP 191568 A1 86.08.20); Ukawa et al., Chem. Pharm. Bull. 33(10):4432-7, 1985; Nohara et al., JP 61010588 A2 (86.01.18); Nohara et al., JP 61010587 A2 (86.01.18); Ihara et al., Yakuri to Chiryo 13(9):4909-22, 1985; Nohara et al., J. Med. Chem. 28(5):559-68, 1985; Nohara, A. and Kuzuna S., EP 102175 A1 (84.03.07); Ishiguro et al., EP 26675 (81.04.08); Ishiguro et al., Heterocycles 16(5):733-40, 1981; Nohara et al., U.S. Pat. No. 4,255,576; Nohara et al., South African Patent ZA 7805054; Ghosh et al., J. Chem. Soc., Perkin Trans. 1(8):1964-8, 1979; Nohara et al., JP 54088298 (79.07.13); Nohara et al., German Patent DE 2841644 (79.04.05); Nohara et al., German Patent DE 2809720 (78.09.14).

The amlexanox compounds are preferably part of a pharmaceutical composition when used in the methods of the present invention. The pharmaceutical composition will include at least one of a pharmaceutically acceptable carrier, diluent or excipient, in addition to one or more amlexanox compoundx and, optionally, other components.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The pharmaceutical compositions that contain one or more amlexanox compounds may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred composition contain, in addition to one or more amlexanox compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of amlexanox compound such that a suitable dosage will be obtained. Typically, this amount is at least 0.01 wt % of an amlexanox compound in the composition. When intended for oral administration, this amount may be varied to be between 0.1 and about 70% of the weight of the composition. Preferred oral compositions contain between about 4% and about 50% of amlexanox compound(s). Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the amlexanox compound of from about 0.1 to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

In the methods of the invention, the amlexanox compound(s) may be administered through use of insert(s), bead(s), timed-release formulation(s), patch(es) or fast-release formulation(s).

It will be evident to those of ordinary skill in the art that the optimal dosage of the amlexanox compound(s) may depend on the weight and physical condition of the patient; on the severity and longevity of the physical condition being treated; on the particular form of the active ingredient, the manner of administration and the composition employed. It is to be understood that use of an amlexanox compound in a chemotherapy can involve such a compound being bound to an agent, for example, a monoclonal or polyclonal antibody, a protein or a liposome, which assist the delivery of said compound.

I. Description of Various Preferred Embodiments

The present invention relates to the discovery that FGF-1 is exported in response to various stimuli, including stressors (such as temperature stress), as an FGF-1 homodimer:synaptotagmin (Syn)-1 complex. This FGF-1:Syn-1 complex may also be cofactor-mediated, as described further hereinbelow. The present invention further discloses that complexation of FGF-1 with Syn-1 may occur under “non-stress” conditions as well and that complexes including FGF-1 and Syn-1 may be involved in processes of FGF-1 export and regulation.

Characterization of FGF-1 as a potent promoter of angiogenesis when elaborated into the extracellular milieu in vivo links FGF-1 to physiologic regulation of FGF-1 export (see, e.g., Folkman et al., Science 235:442-447 (1987)). The present invention provides compositions and methods that are useful for regulating the FGF-1:Syn-1 complex before FGF-1 is released from the cell membrane, or for regulating the protease(s) that allow release of FGF-1 from its complex with Syn-1, thus permitting the controlled export of FGF-1.

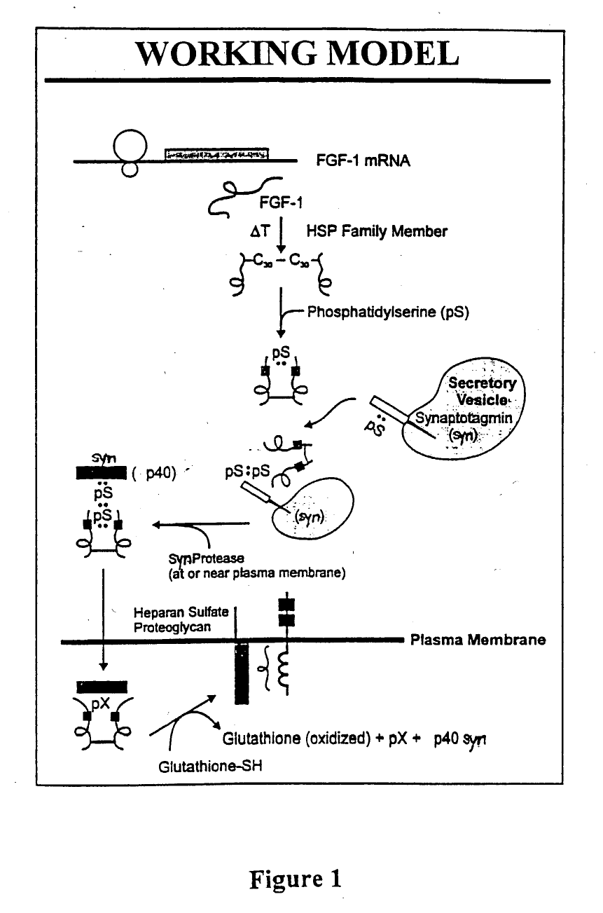

It should be appreciated that the present invention is not limited by the proposed models and mechanisms described herein. Without wishing to be bound by theory, FIG. 1, for example, presents a working model showing a role for FGF-1 in complex formation and including the FGF-1:Syn-1 complex, a mechanism featuring proteolytic cleavage of complex component(s) at or near the cell membrane, and the like, all of which may facilitate an understanding of the invention and are included herein for that purpose.

The existence of an FGF-1:Syn-1 complex, proteolytic cleavage of complex component(s) at or near the cell membrane, and release of the FGF-1 molecule from the cell membrane are all disclosed herein. Various working examples appear below and present data showing the following: (i) the FGF-1 homodimer is associated with a p40 fragment of p65 Syn-1 in medium conditioned by cells subject to heat shock; (ii) expression of antisense to Syn-1 attenuates the release of FGF-1 by cells in response to temperature stress; (iii) mutagenesis of FGF-1 cysteine residues suggests a role for Cys-30 mediated FGF-1 homodimer formation in the release of FGF-1 by cells in response to temperature stress; (iv) immunoprecipitation analysis of extracellular FGF-1 released into medium by NIH-3T3 FGF-1 transfectants in response to heat shock reveals the presence of complexes containing novel FGF-1-associated polypeptides, including an extracellular form of Syn-1; and (v) FGF-1 is present in ovine brain as a low affinity heparin-binding complex of p40 Syn-1:FGF-1 homodimer. The present invention further provides compositions and methods for regulating an FGF-1 export pathway that is distinct from the classical endoplasmic reticulum (ER) and Golgi apparatus mediated protein secretion pathway characterized for secretory proteins that, unlike FGF-1, have leader or “signal” sequences.

As illustrated in FIG. 1, the FGF-1 translation product, in response to temperature, unfolds and undergoes oxidation to form a homodimer specifically utilizing the cysteine residue at position 30 (Cys₃₀) in each subunit. It is important to understand that FGF-1 is not a heat-shock response gene; rather, FIG. 1 illustrates the finding that at 42° C., FGF-1 specifically associates with phosphatidylserine (pS) via its COOH-terminal, heparin-binding domain. (See, e.g., Burgess et al., J. Cell Biol. 111:2129-2138, 1990.) As disclosed herein (See, e.g., Example 10.), the model presented in FIG. 1 is a useful model in other “stress” situations and in “non-stress” situations, as well.

The FGF-1 homodimer:pS complex then may associate with either (i) Syn-1; (ii) the complex of Syn-1 and pS (Syn-1:pS) and/or (i); and/or other proteins. However, according to the model of FIG. 1, it is presumed that FGF-1 utilizes the cytosolically oriented membrane face of the conventional exocytotic pathway. (See, e.g., Rothman, Nature 372:55-63, 1994, and references cited therein.) Release from such constraint (i.e., association of the FGF-1 complex with a membrane surface) may be carried out by a protease that liberates and/or permits solubilization of the FGF-1 homodimer:pS:Syn-1 complex as a cytosolic structure. As used herein, the term “protease” includes native or modified enzymes, proteolytic complexes, or functionally active fragments thereof, whether those fragments are in isolated form or in a complex, as long as the variants or fragments retain proteolytic activity.

Evidence from platelet and red blood cell membrane systems suggests that a “flippase” complex may translocate or “flip” pS between extracellularly and intracellularly oriented membrane locales such as the outer and inner leaflets of the plasma membrane (see, Zachowshi, Biochem. J. 294:1-14, 1993). However, regardless of the mechanism involved, the intracellular FGF-1 complex may traverse the plasma membrane and can then be released as a latent heparin- and FGFR-1-binding protein. Activation by a reducing agent may be required for extracellular FGF-1 to associate with the heparin sulfate proteoglycan or other acidic extracellular matrix macromolecules associated with cell surfaces. Such association may then promote FGF-1 interaction with the high affinity cell surface FGF receptor (FGF-R), through which signal transduction may occur, for example by FGF-R mediated tyrosine phosphorylation.

A. FGF-1

As briefly described above, FGF-1 is a member of a group of proteins that lack a canonical leader or signal sequence, and that are often identified as “leaderless proteins.” As used herein, “leaderless protein” refers to a protein or polypeptide that is found in an extracellular environment but lacks a canonical leader sequence. A leader sequence mediates translocation into the ER and is recognized by signal recognition proteins (SRP). Proteins in the extracellular environment include exported proteins found in extracellular spaces, as well as proteins that are membrane bound, but do not include integral membrane proteins. The prototypic leader sequence has an amino-terminal positively charged region, a central hydrophobic region, and a more polar carboxy-terminal region (see, e.g., von Heijne, J. Membrane Biol. 115:195-201, 1990). Leaderless proteins include FGF-1, FGF-2 (and active fragments thereof), as well as S100, interleukin-1α, interleukin-1β, vas deferens protein, platelet-derived endothelial cell growth factor (PD-ECGF), ciliary neurotrophic factor (CNTF), thymosin, parathymosin, galectin, factor XIIIa, thioredoxin, mammary-derived growth inhibitor, rhodanase, HIV tat, lipocortin and the like. (See, e.g., Muesch et al., Trends In Bioch. Sci. 15:86-88, 1990; Jungnickel et al., FEBS Lett. 346:73-77, 1994; and references cited therein.) Within the context of the present invention, leaderless proteins include naturally occurring proteins, polypeptides or variants thereof that lack signal sequences but are exported by cells, as well as non-naturally occurring or recombinant proteins, polypeptides, or variants thereof including chimeric or fusion proteins, that are engineered to lack a leader sequence but are exported by cells. The terms “signal sequence,” “leader peptide,” and “leader sequence” are used interchangeably herein.

In addition, consistent with usage in the art, FGF-1 is representative of molecules identified as ligand molecules. As used herein, the term “ligand” includes any molecule or portion thereof capable of recognizing (having a binding affinity to) a particular binding molecule. Ligands that recognize a particular binding molecule may exist naturally, or can be prepared as genetically engineered or recombinant molecules, or by synthetic methodologies. Representative ligands may include, but are not limited to, growth factors including FGF-1, cytokines, biotin, antibodies, antigens, hormones, or other molecules capable of specific binding interactions with a binding molecule.

The present invention provides compositions and methods for regulating FGF-1 export from cells, and may offer other related advantages. FGF-1 may be useful in a variety of biological and clinical contexts and has been recognized for potentially beneficial effects in a wide variety of cell and tissue types. (See, e.g., Burgess and Maciag, Annu. Rev. Biochem. 58: 575-606 (1989); Friesel and Maciag, FASEB J. 9: 919-925 (1995); Rifkin and Moscatelli, J. Cell Biol. 109: 1-6 (1989); Klagsbrun and Baird, Cell 67: 229-231 (1991).) In certain embodiments of the present invention, a therapeutically effective amount of an agent that increases FGF-1 export refers to an amount of the agent that stimulates sufficient FGF-1 export to produce levels of extracellular FGF-1 that are effective to bring about a desired beneficial effect. For example, by way of illustration and not limitation, regulated FGF-1 export may provide approaches for promoting the repair and healing of soft tissue injuries including burns, lacerations, and cutaneous ulcerations, as well as injuries to the cornea. The up-regulation of FGF-1 also may promote the healing of bone fractures, torn ligaments, torn or inflamed tendons and inflammation of bursae. Similarly, stimulation of FGF-1 release facilitates the regeneration of cartilage and cartilaginous tissue. FGF-1 also may promote central and peripheral nerve tissue repair and maintenance. Complexes containing FGF-1 and Syn-1 have now been identified in the brain, as discussed further in the Examples that follow. Enhanced FGF-1 release also may have a beneficial or therapeutic effect in the amelioration of vascular injury, as FGF may facilitate vascular tissue repair and the growth of new blood vessels (ie., angiogenesis). Other beneficial effects of increased FGF-1 production are presented in U.S. Pat. Nos. 5,223,483 and 5,401,832, the disclosures of which are hereby incorporated by reference. These and other benefits of regulating FGF-1 export by cells will be appreciated by those familiar with the art and are not intended to be limited by the examples provided herein.

It is one aspect of the present invention to provide methods for regulating FGF-1 export from cells using model cell based systems in vitro. Accordingly, compositions and methods are provided whereby regulation of the FGF-1 export pathway can be achieved, including molecular characterization of export pathway components, functional analysis of agents that potentiate or inhibit FGF-1 export by interacting with pathway components, and manipulation of pathway components to regulate FGF-1 export in a desired fashion.

Determination of FGF-1 export by cells may be accomplished using any method for detecting FGF-1 known in the art. In preferred embodiments the method for detecting FGF-1 further permits quantification of FGF-1 exported by cells. Well known methods for determining FGF-1 in soluble components of cell conditioned media may include (but need not be limited to) immunochemical methods using antibodies that specifically bind to FGF-1, such as radioimmunoassay (RIA), enzyme linked immunosorbent assay (ELISA), immunoprecipitation analysis or “western” immunoblot analysis. Other methods for determining FGF-1 may include biochemical analyses based on known structural and binding properties of FGF such as heparin binding, or biological assays quantifying, for example, cellular responses to FGF-1 present in a sample. These and other methods for determining FGF-1 will be known to those familiar with the art and the examples provided herein shall not be limiting.

Characterization of the FGF-1 export pathway need not be limited to determination of exported FGF-1, but may further comprise identification of molecular components of the pathway. As provided herein, such determination may be accomplished by identification of molecular species that preferentially associate with FGF-1 under defined conditions, for example by co-purifying with FGF-1 in an FGF-1 complex. Because FGF-1 exhibits affinity for heparin, for instance, molecules that co-elute with FGF-1 from immobilized heparin adsorbents may be candidate components of an FGF-1 export pathway complex. Candidate FGF-1 complex components so identified may be further manipulated by a variety of means well known in the art, in order to determine their role in FGF-1 export. Such means include, but are not intended to be limited to, assessment of the effects on FGF-1 export of specific binding agents that interact with candidate complex components, or the effects of genetic manipulation of nucleic acids encoding candidate complex components. Using such model systems, it is within the scope of the instant disclosure to provide a method for regulating FGF-1 export from a cell by regulating the formation of molecular complexes that include FGF-1, where such complexes are formed as a prerequisite to FGF-1 export.

As an example of a model system for regulating FGF-1 export from cells, NIH 3T3 cells transfected with a gene encoding FGF-1 to produce FGF-1-NIH 3T3 transfectants release FGF-1 into culture medium as a response to heat shock(42° C. for two hr), where the FGF-1 is released as a non-heparin-binding and biologically inactive structure (Jackson et al., Proc. Natl. Acad. Sci. USA 89: 10691-10695, 1992) referred to herein as latent FGF-1. In contrast, NIH 3T3 cells transfected with genes encoding either FGF-2 or the IL-1α precursor do not release these transfected gene products into culture media as a response to temperature stress, suggesting that FGF-1 may be preferentially released in response to heat shock by a regulated cellular mechanism.

The heparin-binding and mitogenic activities of latent extracellular FGF-1 may be activated by (NH₄)₂SO₄ fractionation of culture medium conditioned by exposure of 3T3 FGF-1 transfectants to heat shock. Without wishing to be bound by theory, crystallographic data demonstrate the presence of a sulfate-binding site (see Zhu et al., Science 251: 90-93, 1991) in FGF-1, such that occupancy of this site may relate to the mechanism by which (NH₄)₂SO₄ activates latent extracellular FGF-1.

Methods disclosed herein for regulating the release of latent FGF-1 by cells in response to temperature stress may include intervention at the level of either cellular transcription or translation, since the release of latent FGF-1 can be inhibited by including either actinomycin D or cycloheximide in the media of cultured cells exposed to heat shock (Jackson et al., PNAS USA 89: 10691-10695, 1992). Because heat shock alters neither steady-state levels of FGF-1 mRNA nor FGF-1 protein levels in such cells, it appears unlikely that the FGF-1 gene itself (or the pMEXneo expression vector used for transfection) includes a heat shock response element. (Jackson et al., 1992)

B. Synaptotagmin (Syn-1)

According to the present invention, Syn-1 is a component of the FGF-1 export pathway. Syn-1 was initially identified as part of the mechanism utilized by synaptic vesicles to dock at nerve terminal endings (Perin et al., J. Biol. Chem. 266:623-629, 1991). Its structure was deduced from a cDNA clone and predicts a protein (p65) with an NH₂-terminal domain (p40), which was anchored to Golgi vesicles by a transmembrane domain extending into the vesicle by a short COOH-terminal intervesicular domain. An additional structural feature predicted from the cDNA sequence was the apparent lack of a classical signal sequence to direct the transport of Syn-1 into the ER-Golgi apparatus. Furthermore, nine of the ten amino acid residues at the NH₂-terminus of Syn are identical to the NH₂-terminal sequence present in the yeast mating factor “α,” a protein which is released, but which also does not contain a classical signal sequence (Astell et al., Cell 27:15-23, 1981).

The present invention provides compositions and methods that relate to formation of molecular complexes containing FGF-1 and Syn-1 in non-neuronal as well as neuronal cells, and to methods of regulating FGF-1 export from cells that proceeds by a mechanism featuring the formation of such complexes. As disclosed herein, Syn-1 is expressed in non-neuronal cells, including human umbilical vein endothelial cells (HUVEC) and murine NIH 3T3 FGF-1 transfectants. Syn-1 expression in these and other non-neuronal cells may be determined by any of a variety of well known methods for detecting the expression of a specific gene where all or part of the nucleotide sequence encoding the gene is known. For example, Syn-1 expression may be detected using reverse transcription-polymerase chain reaction (RT-PCR). Briefly, RNA may be isolated from cells using established procedures and reverse transcribed to yield cDNA (Garfinkel et al., J. Biol. Chem. 267:24375, 1992). PCR amplification of Syn-1 sequences present in the cDNA template is then performed, for example using the following oligonucleotide primers designed to anneal to both mouse and human Syn-1 sequences:

| | | |
|---|---|---|
| (sense) | 5′-CCATTGCCACCGTGGGCCTT-3′ | SEQ ID NO:9 |
| (antisense) | 5′-TCCAAAACAGTTACCACCAC-3′ | SEQ ID NO:10 |

The identities of amplified PCR products as Syn-1 nucleotide sequences is confirmed by DNA sequencing methodologies that are well known in the art, for example dideoxy sequencing.

Syn-1 expression may be required for cellular FGF-1 release in response to heat shock, and Syn-1 may associate with FGF-1 in high Mᵣ complexes detectable in medium conditioned by cells that have been subjected to temperature stress. Syn-1 encoding nucleic acid sequences have been identified in several different species, including for example, human, rodent and bovine Syn-1 DNA sequences, which are highly conserved (95%).

In one aspect, the present invention provides truncated FGF-1 export pathway component Syn-1 molecules, and in another aspect the invention provides nucleic acids encoding truncated FGF-1 export pathway component Syn-1 molecules. A truncated molecule may be any molecule that comprises less than a full length version of the molecule. Truncated molecules provided by the present invention may include truncated biological polymers, and in preferred embodiments of the invention such truncated molecules may be truncated nucleic acid molecules or truncated polypeptides. Truncated nucleic acid molecules have less than the full length nucleotide sequence of a known or described nucleic acid molecule, where such a known or described nucleic acid molecule may be a naturally occurring, a synthetic or a recombinant nucleic acid molecule, so long as one skilled in the art would regard it as a full length molecule. Thus, for example, truncated nucleic acid molecules that correspond to a gene sequence contain less than the full length gene where the gene comprises coding and non-coding sequences, promoters, enhancers and other regulatory sequences, flanking sequences and the like, and other functional and non-functional sequences that are recognized as part of the gene. In another example, truncated nucleic acid molecules that correspond to a mRNA sequence contain less than the full length mRNA transcript, which may include various translated and non-translated regions as well as other functional and non-functional sequences. In other preferred embodiments, truncated molecules are polypeptides that comprise less than the full length amino acid sequence of a particular protein. As used herein “deletion” has its common meaning as understood by those familiar with the art, and may refer to molecules that lack one or more of a portion of a sequence from either terminus or from a non-terminal region, relative to a corresponding full length molecule, for example, as in the case of truncated molecules provided herein. Truncated molecules that are linear biological polymers such as nucleic acid molecules or polypeptides may have one or more of a deletion from either terminus of the molecule or a deletion from a non-terminal region of the molecule, where such deletions may be deletions of 1-1500 contiguous nucleotide or amino acid residues, preferably 1-500 contiguous nucleotide or amino acid residues and more preferably 1-300 contiguous nucleotide or amino acid residues. In certain particularly preferred embodiments truncated nucleic acid molecules may have a deletion of 270-330 contiguous nucleotides. In certain other particularly preferred embodiments truncated polypeptide molecules may have a deletion of 80-140 contiguous amino acids.

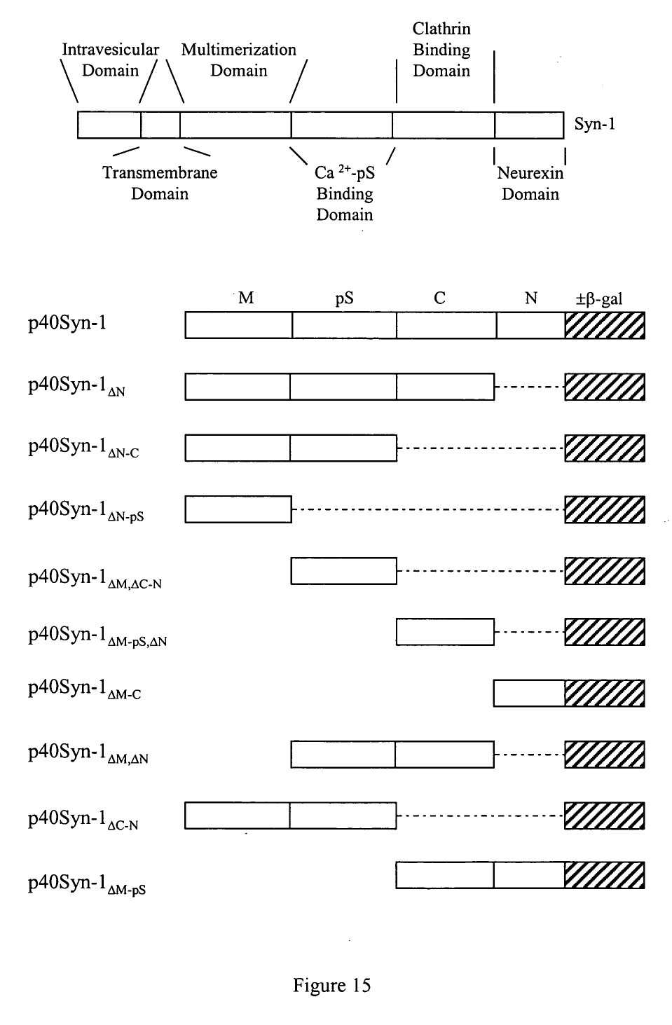

Analysis of Syn-1 truncation deletion mutants provides evidence that truncated Syn-1 proteins according to the present invention may be divided into structural domains that are responsible for particular Syn-1 functional properties. For example, clathrin binding and calcium/phosphatidylserine binding activities reside in distinct structural regions of the Syn-1 molecule. (See, e.g., Zhang et al., Cell 78:751-760, 1994; Perin et al., Nature 345:260-263, 1990.) (FIG. 15).

The use of Syn-1 truncation deletion mutants permits molecular fine regulation of FGF-1 export from a cell as a function of the particular Syn-1 domain that is affected by the deletion, in contrast to molecular regulation of FGF-1 export by other mechanisms provided herein, where either full length Syn-1 is overexpressed, or Syn-1 expression is impaired. As disclosed below using transfected cell lines capable of expressing FGF-1, antisense Syn-1 expression may inhibit Syn-1 expression and lead to impairment of FGF-1 export in response to heat shock. Alternatively, as also provided by the present invention, Syn-1 overexpression in stable FGF-1 transfectants may result in increased FGF-1 release following heat shock. Accordingly, in certain embodiments of the invention, cellular responses to heat shock that produce detectable increases or decreases in FGF-1 export by cells transfected with Syn-1 truncation deletion mutants may permit correlation of a particular Syn-1 structural domain with a particular FGF-1 regulating effect. For example, a domain deletion strategy as set forth in FIG. 15 may be used to determine structure-function relationships within the Syn-1 molecule. As an alternative approach, for example, FGF-1:β-gal 3T3 cell transfectants may be co-transfected with either the full length sense strand DNA encoding Syn-1 open reading frame, or with the truncated sense strand encoding only the p40 Syn-1 open reading frame, to compare the temperature-induced FGF-1 export response in these cells.

According to the present invention, one or more components of complexes that are required for FGF-1 export may include a membrane binding site. Components having a membrane binding site may be full length or truncated FGF-1, Syn-1, S100A13 or other components. Membrane binding sites of such components may be regions, portions, domains, structures, motifs, determinants or the like that are capable of mediating binding interactions with membranes, and membrane binding sites may be either formed within individual complex components or may result from direct or indirect interactions among more than one complex component. Accordingly, more than one molecular component of a complex may contribute structurally to the membrane binding site, or alternatively, a component molecule of the complex may not include any part of the membrane binding site but may influence the conformation of molecular components that do form the binding site.

Membrane binding sites may interact with any cellular membrane, and typically interact with membranes that are in contact with cytosolic components, including the cytoplasmic aspects of intracellular membrane bounded compartments such as vesicles, ER-Golgi constituents, organelles and the like, as well as the cytoplasmic aspect of the plasma membrane. In preferred embodiments, a membrane binding site in a complex binds to a cytoplasmically accessible component of a plasma membrane, which refers to intracellularly oriented plasma membrane components that are associated with the inner leaflet phospholipid bilayer and that are available under physiological conditions for interaction with cytosolic or other cytoplasmic components. In another preferred embodiment, a membrane binding site is a lipid binding site, and in a more preferred embodiment a membrane binding site is a phospholipid binding site. In a particularly preferred embodiment, a membrane binding site is a phosphatidlyserine (pS) binding site. Phosphatidylserine is a negatively charged phospholipid that typically exhibits a restricted subcellular distribution, being localized to the cytoplasmically disposed or inner leaflet of the plasma membrane. (See, e.g., Cullis et al., Biochem. Biophys. Acta 559:399, 1979.) Phosphatidylserine binding sites that occur in proteins are known in the art and include, for example, pS binding sites of FGF-1 (Burgess et al., J. Cell Biol. 1 11:2129, 1990) and annexin (Moss et al., in Novel Calcium Binding Proteins, Springer Verlag, p. 535, 1991).

While it is expected that the expression of an “appropriate” truncated Syn-1 domain deletion mutant will attenuate the release of FGF-1, the degree of attenuation may not be as pronounced as that described herein using the γ-Syn-1 antisense expression plasmid. Therefore, two strategies have been designed to ascertain whether the FGF-1 export pathway is functioning near saturation, or if not, if there are insufficient intracellular levels of the individual truncated Syn-1 domain deletion mutant molecules to overcome the activity of endogenous full length Syn-1 protein. The approaches disclosed herein (i) employ the use of co-transfected cell lines in which an FGF-1 encoding construct can be stably expressed along with both a Syn-1 antisense construct (γ-Syn-1; AUG start site oriented γ-Syn-1 construct) to inhibit endogenous Syn-1 expression and a sense Syn-1 domain deletion mutant construct to direct expression of a truncated Syn-1; and (ii) allow one to obtain stable FGF-1:β-gal 3T3 transfectants that have been co-transfected with a construct encoding the appropriate truncated Syn-1 domain.

The Syn-1 domain deletion strategy may be supplemented by an independent point mutagenesis study in which the role of the Syn-1 cleaving enzyme (Syn-1-CE) cleavage site in the FGF-1 release pathway is assessed, in order to determine whether a Syn-1 point mutant may function as a dominant negative regulator of FGF-1 release in response to temperature stress. Those skilled in the art will be familiar with methodologies for generating and/or detecting Syn-1 point mutations, and for correlating the presence of such mutations with altered proteolysis of Syn-1 by Syn-1-CE. For example by way of illustration and not limitation, point mutations in Syn-1 encoding nucleotide codons that encode amino acids at sites of Syn-1 cleavage by Syn-1-CE may generate Syn-1 mutants that are protease resistant. As provided by the instant disclosure, such protease resistance may profoundly alter the activity of Syn-1 in the FGF-1 export pathway, thus providing a method of regulating FGF-1 export by altering the properties of FGF-1 :Syn-1 complexes.

C. Other Members of the FGF-1:Syn-1 Complex

It is another aspect of the invention to provide a method of identifying additional components of the FGF-1:Syn-1 complex, the formation of which is required for FGF-1 export. According to this aspect of the invention, FGF-1 and additional molecular species (which may include but need not be limited to Syn-1 and/or S100A13) that preferentially associate with FGF-1 may be isolated in the form of a molecular complex based on the present findings that formation of such a multi-component complex precedes and is required for FGF-1 export. Once isolated, the complex may be analyzed by any biochemical, physical or chemical method known to those familiar with the art, in order to characterize the molecular species that are components of the complex, in order to determine the presence of a molecular species that preferentially associates with FGF-1.

Isolation of a complex comprising FGF-1 refers to physical separation of such a complex from its biological source, and may be accomplished by any of a number of well known techniques including but not limited to those described herein, and in the cited references. Once so isolated, the complex comprising FGF-1 may be analyzed to determine the presence of molecular species that preferentially associate with FGF-1 in the complex. Without wishing to be bound by theory, a molecular species that “preferentially associates” with FGF-1 can be any discrete molecular component that may, but need not, directly bind to FGF-1, and may in the alternative bind indirectly to FGF-1 by interacting with one or more additional components that either bind to FGF-1 or interact with still other complex components that bind to FGF-1. These or other mechanisms by which a component may preferentially associate with FGF-1 are within the scope of the claimed methods, so long as isolating a complex comprising FGF-1 also results in isolation of the molecular species that further comprise such a complex.

In particularly preferred embodiments of the invention, a complex comprising FGF-1 may be isolated either prior to or following export of the complex from a cell, where such a cell is capable of producing FGF-1 by expressing a product of an FGF-1 encoding nucleic acid sequence, and where such a cell is further capable of exporting FGF-1 according to criteria described herein. In certain preferred embodiments, the complex comprising FGF-1 may be isolated from the medium conditioned by cells that export FGF-1, following export of the complex from such cells. In certain other embodiments the complex comprising FGF-1 may be isolated prior to export by cells, or from cultured cells. In still other embodiments the complex comprising FGF-1 may be isolated from a biological source that is a biological tissue, for example brain tissue. It is understood that complexes comprising FGF-1 that may be isolated from a biological tissue may include complexes that have been exported from cells and complexes that have not been exported from cells, and these embodiments of the invention are not to be limited according to the export status of a complex comprising FGF-1. Similarly, while in certain embodiments of the invention the complex comprising FGF-1 may further comprise dimeric FGF-1, other embodiments of the invention as they relate to FGF-1 need not be so limited.

As disclosed herein, a complex comprising FGF-1 may include FGF-1 that is the product of an endogenous gene, and may also include FGF-1 that is the product of a transfected gene, which may further include nucleic acid sequences encoding truncated FGF-1 molecules. An “endogenous gene” refers to a gene that is naturally present in the cell from which a complex comprising FGF-1 is isolated, and a “transfected gene” refers to any nucleic acid sequence that is introduced into a cell and expressed therein. In preferred embodiments of the present invention, transfected genes may encode FGF-1 that is present in complexes, and in particularly preferred embodiments transfected genes may encode truncated FGF-1 as described above. For example by way of illustration and not limitation, truncated FGF-1 comprising FGF-1₍₂₁₋₁₅₄₎ lacking amino acids 1-20 of full length human FGF-1, or FGF-1₍₁₅₋₁₅₄₎ having an N-terminal deletion of 14 amino acids relative to the full length human FGF-1 amino acid sequence, may be particularly useful. Those familiar with the art readily appreciate that various other nucleic acid constructs encoding truncated FGF-1 may be useful for transfecting particular cells in order to practice the invention.

Techniques for isolating a complex comprising FGF-1 may include any biological and/or biochemical methods useful for separating the complex from its biological source. Those familiar with the art will be able to select an appropriate method depending on the biological starting material and other factors. Such methods may include, but need not be limited to, cell fractionation, density sedimentation, differential extraction, salt precipitation, ultrafiltration, gel filtration, ion-exchange chromatography, partition chromatography, hydrophobic chromatography, electrophoresis, affinity techniques or any other suitable separation method.

Affinity techniques are particularly useful in the context of the present invention, and may include any method that exploits a specific binding interaction with a component of a complex comprising FGF-1 to effect a separation. For example, because FGF-1 contains a heparin binding domain, an affinity technique such as binding of a complex comprising FGF-1 to immobilized heparin under conditions that preserve the complex may be a particularly useful affinity technique. Other useful affinity techniques include immunological techniques for isolating complexes comprising FGF-1, which techniques rely on specific binding interaction between antibody combining sites for antigen and antigenic determinants present in the complexes. Immunological techniques include, but need not be limited to, immunoaffinity chromatography, immunoprecipitation, solid phase immunoadsorption or other immunoaffinity methods. See, for example, Scopes, R. K., Protein Purification: Principles and Practice, 1987, Springer-Verlag, NY; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques.

As described above, methods for the determination of the presence of FGF-1 are well known in the art. Additionally, the occurrence of FGF-1 in a molecular complex having other components can be determined by a variety of techniques known to those of ordinary skill in the art. For example, determination of the presence of complexes may be made using procedures such as sedimentation, gel electrophoresis, co-purification, immunoblot detection or co-immunoprecipitation, or other techniques, including characterization of additional molecular species present in a complex by physicochemical properties such as spectrometric absorbance, molecular size and/or charge, solubility, peptide mapping, sequence analysis and the like. (See, e.g, Engleka et al., 1992 J. Biol. Chem. 267:11307, which is incorporated by reference, and other references cited above.)

Accordingly, additional separation steps for biomolecules may be optionally employed to further identify molecular species that preferentially associate with FGF-1 in complexes. These are well known in the art and may include any separation methodology for the isolation of proteins, lipids, nucleic acids or carbohydrates, typically based on physicochemical properties of the newly identified components of the complex. Examples of such methods include RP-HPLC, ion exchange chromatography, hydrophobic interaction chromatography, hydroxyapatite chromatography, native and/or denaturing one- and two-dimensional electrophoresis, ultrafiltration, capillary electrophoresis, substrate affinity chromatography, immunoaffinity chromatography, partition chromatography or any other useful separation method. For example, sufficient FGF-1 complex protein may be obtained by for at least partial structural characterization of one or more complex protein components by microsequencing. Using the sequence data so generated, any of a variety of well known suitable strategies for further characterizing the additional protein components present in FGF-1 containing complexes may be employed. For example, nucleic acid probes may be synthesized for screening one or more human cDNA libraries to detect, isolate and characterize a cDNA encoding such complex component(s). Other examples may include use of the partial sequence data in additional screening contexts that are well known in the art for obtaining additional amino acid and/or nucleotide sequence information for the identified FGF-1 complex component. See, e.g., Molecular Cloning: A Laboratory Manual, second edition, edited by Sambrook, Fritsch & Maniatis, Cold Spring Harbor Laboratory, 1989, and the revised third edition thereof. Such approaches may further include nucleic acid library screening based on expression of library sequences as polypeptides, such as binding of such polypeptides to antibodies reactive with FGF-1 complex components, phage display screening approaches or dihybrid screening systems based on protein-protein interactions, and the like, any or which may be adapted to screening for FGF-1 complex components provided by the present invention using routine methodologies with which those having ordinary skill in the art will be familiar. (See, e.g., Bartel et al., In Cellular Interactions in Development: A Practical Approach, Ed. D. A. Harley, 1993 Oxford University Press, Oxford, UK, pp. 153-179, and references cited therein; Parmley et al. 1988 Gene 73:305 and references cited therein; and Katti et al., 1990 J. Mol. Biol. 212:167 and references cited therein; all of which are hereby incorporated by reference.) Preferably extracts of cell conditioned medium, cultured cells, and in particularly preferred embodiments extracts of biological tissues or organs have been shown to be a reliable and stable source of novel FGF-1 complex proteins (e.g., Syn-1, FGF-1), with brain tissue being a further particularly preferred source.

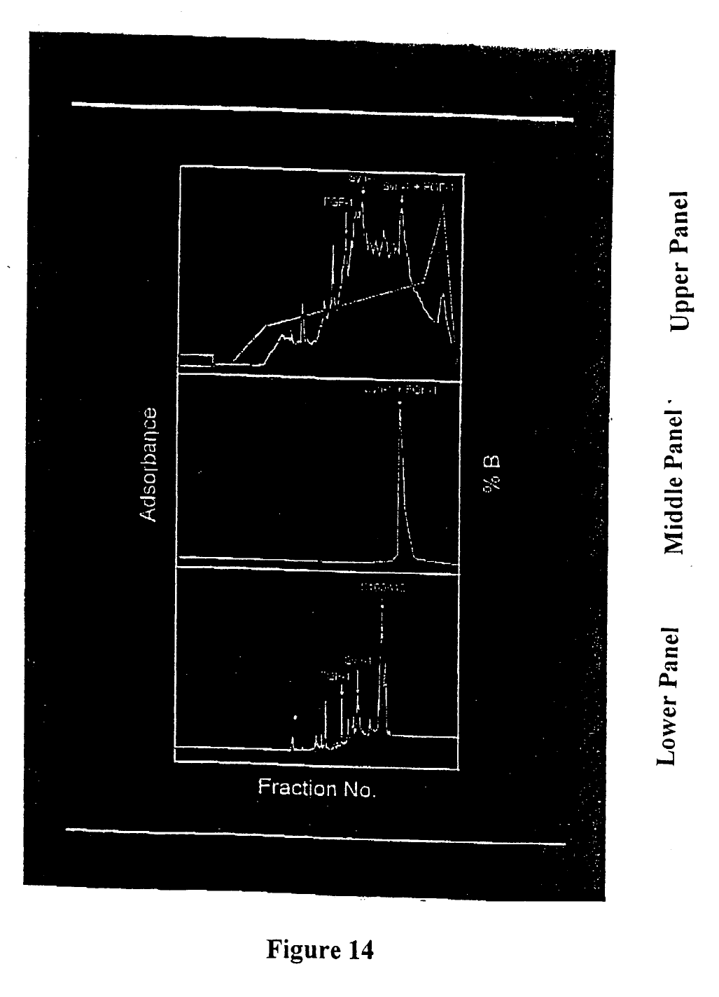

For example, FGF-1:Syn-1 complexes containing additional molecular species may be isolated by heparin affinity chromatography from tissue/organ extracts prepared at neutral pH, and heparin binding material may be treated with denaturing agents. Resolution of such a preparation using reversed phase high performance liquid chromatography (RP-HPLC) may generate multiple additional peaks representing distinct proteins having different elution retention times. Thus, treatment of a tissue preparation comprising the FGF-1:Syn-1 complex with a chaotropic agent (e.g., 8M guanidinium HCl) may be used to demonstrate that in neutral pH extracts of a suitable tissue, FGF-1 is present in complexes that further comprise Syn-1 and other proteins. In certain preferred embodiments of the present invention the biological source for such complexes may be brain tissue. As described herein, these complexes may be unusual among multicomponent molecular complexes containing proteins in that they may be remarkably stable to solvents commonly used to resolve proteins in reverse-phase high performance liquid chromatography (RP-HPLC). See, e.g, Example 9 and FIG. 14.

1. Ovine S100A13 Homolog

Since the identity of the individual protein components of the FGF-1:Syn-1 complex could not be accessed by direct NH₂-terminal sequence analysis, peptide maps of those peaks exhibiting prominent absorbance profiles were obtained. Analysis of the most prominent peaks by NH₂-terminal sequence analysis of multiple proteinase K-derived peptides identified the first peak to be identical to the ovine homolog of human S100A13. (For background on S100A13, see, e.g., Wicki et al., 1996 Biochem. Biophys. Res. Commun. 227:594; and Schafer et al., 1996 Trends Biochem. Sci. 21:134, which are hereby incorporated by reference in their entireties.) Similar analysis of the second prominent absorbance peak demonstrated it to be identical to the ovine homolog of human cyclophilin B. Thus, the present data clearly identifies S100A13 and cyclophilin B to be components of the FGF-1:Syn-1 complex.

S100A13 is a recently identified member of the human S100A gene family with well conserved structural features. (Wicki et al., 1996; Schafer et al., 1996.) Characterization of the isolated S100A13 gene has shown that it contains the prerequisite structural motifs identifying the S100A gene family members as Ca²⁺-, calmodulin-, and acidic phospholipid-binding proteins. Interestingly, S100A proteins have been implicated as chemotactic and neurotrophic agents, which are activities also associated with FGF-1. Although it is known that, like FGF-1, S100A proteins are able to modify intracellular tyrosine phosphorylation (MAPK-dependent) and transcriptional events, no known receptor for any of the S100A family members has been identified. Further, S100A gene expression is diagnostic for metastatic human melanoma. While FGF-1 is well characterized as a mitogen for human melanocytes, its secretion has been implicated in the regulation of human metastatic tumor potential due to its ability to stimulate angiogenesis in vivo.

In addition, S100A13 is a cysteine-free structure that is exported by cells despite the fact that, like FGF-1, it lacks a classical signal peptide sequence to direct it through the conventional ER-Golgi mediated protein secretory pathway. Nevertheless, S100A proteins are well characterized as calmodulin binding proteins, and are involved in the regulation of the filamentous cytoskeleton. As disclosed elsewhere herein, in the non-classical FGF-1 export pathway for leaderless proteins, FGF-1 may interact with the cytosolic aspect of conventional Syn-1 containing cytoplasmic vesicles en route to the inner surface of the plasma membrane. S100A 13 may serve as a conduit between the exocytotic organelle and the calmodulin-rich F-actin cytoskeleton. In addition, observations disclosed herein suggest that (i) the biologic activities previously attributed to S100A gene family members may actually reside in an FGF gene family member present as a contaminant in S100A preparations, and (ii) S100A antagonists may be used to limit the release of FGF-1 from cells via the FGF-1 export pathway.

2. Cyclophilin B

As disclosed herein, Cyclophilin B (CpB) is identified as a component of the ovine brain-derived FGF-1:Syn-1 complex. CpB is a cyclosporin A1 (CSA)-binding protein, which was initially identified as a heat shock-induced peptidylproline cis-trans isomerase (hsp40) involved in protein folding and protein-protein interactions. CsA is well recognized as an immunosuppressant in humans, and gives rise to unusual tissue/organ-specific side effects, including enhanced peripheral nerve repair. FGF-1 is also a potent neurotropic agent in vivo, in addition to its well recognized role as a cell survival factor.

Different cyclophilins are located in different compartments of the cell. Cyclophilin B, which has a peptide signal sequence, is known to reside in the ER. However, cyclophilin B may traffic through the ER-pre-Golgi pathway in association with secreted proteins inside secretory vesicles. Therefore, cyclophilin B isolated from the brain as part of the FGF-1:Syn-1 complex may associate with the intravesicular portion of Syn-1, and may be able to traffic with the vesicle associated complex. Cyclophilin in this context may be necessary to regulate the assembly of the Syn-1 monomer into a dimeric or multimeric structure, to regulate and/or direct the traffic of the secretory vesicles under stress conditions, or to control the proteolytic formation of the p40 fragment from p65 Syn-1 . Regardless of the precise mechanism by which CpB may be related to the FGF-1 export pathway, according to the present invention FGF-1 complex formation is a prerequisite to FGF-1 export. Accordingly, the presence of CpB as well as Syn-1 in such complexes places CpB and agents that influence CpB participation in complex formation within the scope of the methods for regulating FGF-1 export provided by the invention.

According to the instant disclosure, Syn-1 and FGF-1 associate as a multiprotein complex in neural tissue in vivo, and in non-neural cell based in vitro systems. In addition, because two additional proteins present within the neural cell derived FGF-1:Syn-1 complex have been identified as S100A13 and cyclophilin B, agents that promote or inhibit this association may be useful to potentiate or impair the FGF-1 export pathway in vitro and in vivo.

D. Syn-1 Protease

In another embodiment of the invention, the protease responsible for cleaving and releasing Syn-1 into the conditioned medium of heat-shocked NIH 3T3 cells as a proteolytic fragment (p40) of the p65 Syn-1 precursor is disclosed. Although not intended to be limiting, preferred FGF-1 constructs for demonstrating the proteolytic effect are the FGF-1₁₋₁₅₄ and FGF-1₁₋₁₅₄:β-gal 3T3 cell transfectants; similar constructs may also be prepared to generate endothelial cell (EC) and smooth muscle cell (SMC) transfectants. This enzyme is of particular interest because of its ability to cleave the cytosolic domain of Syn-1 on the extravesicular side of the transmembrane sequence, which results in the formation of a soluble cytosol-derived FGF-1 secretory complex.

Once the active site character of the protease is known, conventional methods, using e.g., fluorescent esters and amides as alternative substrates for the protease, will permit the elucidation of its kinetic parameters, as well as permit the definition of an enzyme unit for the determination of specific activity. A combination of protease inhibitor affinity chromatography, heparin-Sepharose™ chromatography, and reversed phase HPLC methods is capable of providing sufficient protein with high specific activity for microsequence analysis, which will provide direct access to the cDNA encoding the protease, for example, through conventional cDNA cloning using the λgt11 HUVEC/NIH 3T3 cell libraries as described (Zhan et al., J. Biol. Chem. 268:24427-24431 (1993); Zhan et al., J. Biol. Chem. 269:20221-20224, 1994).

Identification of the peptide bond(s) in Syn-1 cleaved by the protease may be readily performed by sequence analysis of the p40 product of the Syn-1 (p65) precursor in the protease reaction mixture, since the primary structure of Syn-1 is known. For example by way of illustration and not limitation, a trypsin cleavage site is situated between Syn-1 amino acids 111 and 112, and any of a number of additional basic amino acid residues upstream from this site may also be protease cleavage sites.

Should the cell culture-based strategy for the purification of the Syn-1-cleaving enzyme (Syn-1-CE) prove to be cumbersome, an alternative method is available using bovine brain as a starting material from which to purify and characterize the protease, as described by Maciag et al. (J. Biol. Chem. 257:5333-5336, 1982). High Mᵣ FGF-1 fractions are collected using gel exclusion chromatography, incubated at pH 4.0 to convert the high Mᵣ form of FGF-1 to its conventional low Mᵣ (20 kDa) form, and the low Mᵣ form of FGF-1 is resolved by gel exclusion chromatography and isoelectric focusing. According to this approach, the high Mᵣ form of FGF-1 may contain FGF-1 complexed to Syn-1 as an FGF-1 homodimer (see, e.g., Example 9).

Heparin-Sepharose™ unbound and bound fractions, the latter being eluted with a salt gradient as described below, are screened for the presence of a fraction(s) containing Syn-1 cleavage enzyme (Syn-1-CE) by monitoring proteolytic conversion of [¹²⁵I]p65-Syn-1 to [¹²⁵I]p40-Syn-1 using standard enzymatic assay methodologies that are well known in the art, including for example discrimination between p65 Syn-1 and p40 Syn-1 by gel electrophoresis. Additional protein separation steps may be optionally employed to further isolate active Syn-1-CE; these are well known in the art and may include any separation methodology for the isolation of proteins, typically based on physicochemical properties of the enzyme. Examples of such methods include RP-HPLC, ion exchange chromatography, hydrophobic interaction chromatography, hydroxyapatite chromatography, native and/or denaturing one- and two-dimensional electrophoresis, ultrafiltration, capillary electrophoresis, substrate affinity chromatography, immunoaffinity chromatography or any other useful separation method. Sufficient Syn-1-CE protein is obtained by for at least partial structural characterization by microsequencing, after which a human brain stem cDNA library is screened to isolate and characterize the Syn-1-CE cDNA. Organ extracts have been shown to be a reliable and stable source of novel proteins (i.e., Syn-1, FGF-1, etc.).

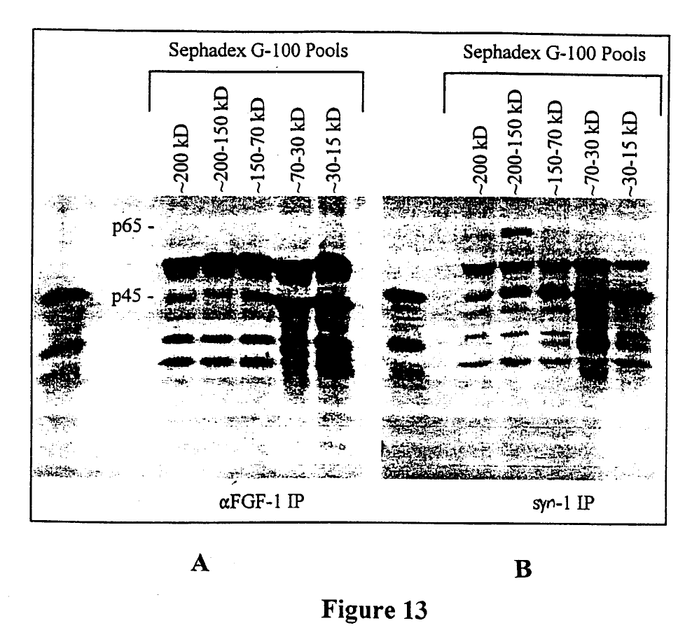

An FGF-1 antibody was able to resolve p40 Syn-1 and low Mᵣ Syn-1 fragments (see, FIG. 13), but not p65 Syn-1 by Syn-1 immunoblot analysis. In contrast, while p65 Syn-1 was readily resolved by Syn-1 immunoprecipitation followed by Syn-1 immunoblot analysis in the high Mᵣ post-Sephadex G-100 pools, p65 Syn-1 was not detected in the high Mᵣ post-Sephadex G-100 pools by FGF-1 immunoprecipitation (FIG. 13). Thus, it is possible that the FGF-1 homodimer may not be able to associate with p65 Syn-1, but may associate with p40 Syn-1.

Moreover, since both FGF-1 and Syn-1 immunoprecipitates were able to resolve lower Mᵣ Syn-1 fragments (p29 and p35) and the lowest Mᵣ Syn-1 fragment, p29, it is possible that the Syn-1 fragment may define the minimal Syn-1 sequence able to associate with the FGF-1 homodimer. To confirm this, p29 Syn-1 may be purified, for example from ovine brain using a combination of ion exchange and reversed phase HPLC techniques, analyzed by Syn-1 immunoblot and conventional silver stained SDS-PAGE analysis, and the NH₂-terminal Syn-1 protein sequenced. CNBr and trypsin p29 Syn-1 fragments may be resolved by e.g., microbore HPLC, and the fragments sequenced.

1. Syn-1 Protease Activity Induced by Stress

According to the methods of regulating FGF-1 export disclosed herein, a cellular response to heat shock characterized by the export of FGF-1 may involve a mechanism whereby formation of a complex comprising FGF-1 and Syn-1 is a prerequisite to FGF-1 export, and further whereby a catalytic event involving specific proteolysis of Syn-1 may be the result of a cytosolic protease activity induced by heat shock. As described below, the presence of a specific protease cleavage site in the extravesicular domain of Syn-1 may be required for FGF-1 release from cells following heat shock. When the Syn-1 extravesicular cytoplasmic domain is present intracellularly, heat shock induced specific protease activity (Syn-1 cleaving enzyme) generates a p40 Syn-1 fragment that is found associated with exported, extracellular FGF-1 in the medium of cells exposed to heat shock. If Syn-1 truncation deletion mutants lacking the specific extravesicular domain protease cleavage site are present in FGF-1 expressing cells, FGF-1 export as a cellular response to heat shock does not occur.

Accordingly, the present invention provides compositions and methods for regulating FGF-1 export from cells. In certain embodiments of the invention, FGF-1 export may be regulated by regulating the activity of the Syn-1 cleaving enzyme (Syn-1-CE). Regulation of Syn-1-CE may be by any of a variety of well known methods for specifically inhibiting proteases, including but not limited to administration of specific Syn-1-CE inhibitors, or engineering mutant Syn-1 molecules that are resistant to Syn-1-CE mediated proteolysis by virtue of having altered amino acid sequences at the protease recognition site(s). As also described below, inhibition of the FGF-1 export pathway response to heat shock may also be realized by inhibition of Syn-1 expression using Syn-1 antisense constructs.

Regulation of FGF-1 export may also take the form of promoting Syn-1-CE activity. Without wishing to be bound by theory, Syn-1-CE activity may be potentiated by a variety of methods, including but not limited to overexpressing Syn-1-CE, genetically engineering a mutant Syn-1-CE having enhanced catalytic activity, and engineering a mutant Syn-1 that exhibits increased sensitivity to Syn-1-CE.

E. Cell Transfections and Synthesis of Proteins

DNA molecules composed of a protein gene or a portion thereof, can be operably linked into an expression vector and introduced into a host cell to enable the expression of these proteins by that cell. Alternatively, a protein may be cloned in viral hosts by introducing the “hybrid” gene operably linked to a promoter into the viral genome. The protein may then be expressed by replicating such a recombinant virus in a susceptible host. In certain embodiments of the present invention, a host cell is transfected with two or more DNA constructs encoding distinct gene products, the expression of which by the host cell is desired. Such cells are referred to herein as “co-transfectants”.

A DNA sequence encoding a protein molecule may be recombined with vector DNA in accordance with conventional techniques. When expressing the protein molecule in a virus, the hybrid gene may be introduced into the viral genome by techniques well known in the art. For example, cloning in filamentous phage is generally described in Sambrook et al., supra. Thus, the present invention encompasses the expression of the desired proteins in either prokaryotic or eukaryotic cells, or viruses which replicate in prokaryotic or eukaryotic cells. Preferably, the proteins of the present invention are cloned and expressed in a virus.

Viral hosts for expression of the proteins of the present invention include viral particles which replicate in prokaryotic host or viral particles which infect and replicate in eukaryotic hosts. Preferably, viruses which replicate in prokaryotic host are used according to the present invention. Illustrative prokaryotic viruses include, but are not limited to, the Ff class of filamentous phage (fd, f1, M13, etc.), bacteriophage λ, bacteriophage T4, and bacteriophage T3. The preferred phage for expressing a protein is the Ff class of filamentous phage (including strains f1, fd and M13), as summarized by G. P. Smith (Current Opinion in Biotechnology, Vol. 2/5, October 1991). “Phage” as used herein also includes “phagemids,” which are plasmids containing DNA sequences that allow the plasmid DNA to be packaged into filamentous phage particles when the plasmid-bearing cell receives an appropriate helper genome (e.g. Mead and Kemper, in Vectors: A Survey of Molecular Cloning Vectors and Their Uses, edited by R. Rodriquez and D. T. Denhardt, Butterworths, 1987, pp. 103-111). Examples of eukaryotic viruses include adenovirus, bovine papilloma virus, simian virus, tobacco mosaic virus and the like.

Expression of the desired protein in a viral host may be accomplished by operably linking the “hybrid” gene encoding a protein to a viral promoter. Alternatively, a promoter from the host in which the virus replicates may also be used. Preferably, the viral surface protein promoter is used to express the protein in the viral host. For example, the DNA molecule encoding a binding molecule can be inserted directly at any location within the surface protein gene. As long as the insertion maintains the correct reading frame for both the binding molecule and the surface protein genes (or portions of these genes), it is possible to express the proteins of the present invention in a viral microorganism. As used herein, the term “binding molecule” refers to a protein or portion thereof which binds to another protein or portion thereof, which is usually termed a ligand.

The expression of the proteins of the present invention can also be accomplished in procaryotic cells. Preferred prokaryotic hosts include bacteria, such as E. coli, Bacillus, Streptomyces, Pseudomonas, Salmonella, Serratia. The most preferred prokaryotic host is E. coli.

To express the desired protein in a prokaryotic cell (such as, for example, E. coli, B. subtilis, Pseudomonas, Streptomyces, etc.), it is necessary to operably link the desired protein encoding sequence to a functional prokaryotic promoter. Such promoters may be either constitutive or regulatable (i.e., inducible or derepressible). Examples of constitutive promoters include the int promoter of bacteriophage λ, and the bla promoter of the β-lactamase gene of pBR322, etc. Examples of inducible prokaryotic promoters include the major right and left promoters of bacteriophage λ (P_{L} and P_{R}), the trp, recA, lacZ, lacI, gal, and tac promoters of E. coli, the a-amylase (Ulmanen et al., J. Bacteriol. 162:176-182 (1985)), and the s-28-specific promoters of B. subtilis (Gilman, et al., Gene 32:11-20 (1984)), the promoters of the bacteriophages of Bacillus (Gryczan, T. J., In: The Molecular Biology of the Bacilli, Academic Press, Inc., NY (1982)), arid Streptomyces promoters (Ward et al., Mol. Gen. Genet. 203:468-478 (1986)). Prokaryotic promoters are reviewed by Glick, B. R., (J. Ind. Microbiol. 1:277-282 (1987)); Cenatiempo, Y. (Biochimie 68:505-516 (1986)); and Gottesman, S. (Ann. Rev. Genet. 18:415-442 (1984)). Proper expression in a prokaryotic cell also requires the presence of a ribosome binding site upstream from the protein-encoding sequence. Such ribosome binding sites are disclosed, for example, by Gold et al. (Ann. Rev. Microbiol. 35:365-404 (1981)).

Eukaryotic hosts for cloning and expression of the proteins of the present invention include insect cells, yeast (especially Saccharomyces), fungi (especially Aspergillus), and mammalian cells (such as, for example, human or primate cells) either in vivo, or in tissue culture.

The expression of the desired protein in eukaryotic hosts requires the use of eukaryotic regulatory regions. Such regions will, in general, include a promoter region sufficient to direct the initiation of RNA synthesis. Preferred eukaryotic promoters include the promoter of the mouse metallothionein I gene (Hamer et al., J. Mol. Appl. Gen. 1:273-288 (1982)); the TK promoter of Herpes virus (McKnight, S., Cell 31:355-365 (1982)); the SV40 early promoter (Benoist et al., Nature (London) 290:304-310 (1981)); the yeast gal4 gene promoter (Johnston et al., Proc. Natl. Acad. Sci. (USA) 79:6971-6975 (1982); Silver et al., Proc. Natl. Acad Sci. (USA) 81:5951-5955 (1984)). As is widely known, translation of eukaryotic mRNA is initiated at the codon which encodes the first methionine. For this reason, it is preferable to ensure that the linkage between a eukaryotic promoter and a DNA sequence which encodes the desired protein does not contain any intervening codons which are capable of encoding a methionine (i.e., AUG).

The desired protein encoding sequence and an operably linked promoter may be introduced into a recipient prokaryotic or eukaryotic cell either as a non-replicating DNA (or RNA) molecule, which may either be a linear molecule or, more preferably, a closed covalent circular molecule. Since such molecules are incapable of autonomous replication, the expression of the desired protein may occur through the transient expression of the introduced sequence. Alternatively, permanent expression may occur through the integration of the introduced sequence into the host chromosome. For expression of the desired protein in a virus, the hybrid gene operably linked to a promoter is typically integrated into the viral genome, be it RNA or DNA. Cloning into viruses is well known and thus one of skill in the art will know numerous techniques to accomplish such cloning.

Cells which have stably integrated the introduced DNA into their chromosomes can be selected by also introducing one or more markers which allow for selection of host cells which contain the expression vector. The marker may complement an auxotrophy in the host (such as leu2, or ura3, which are common yeast auxotrophic markers), biocide resistance, e.g., antibiotics, or resistance to heavy metals, such as copper, or the like. The selectable marker gene can either be directly linked to the DNA gene sequences to be expressed, or introduced into the same cell by co-transfection.

In another embodiment, the introduced sequence will be incorporated into a plasmid or viral vector capable of autonomous replication in the recipient host cell. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

For a mammalian host, several possible vector systems are available for expression. One class of vectors utilize DNA elements which provide autonomously replicating extra-chromosomal plasmids, derived from animal viruses such as bovine papilloma virus, polyoma virus, adenovirus, or SV40 virus. A second class of vectors relies upon the integration of the desired gene sequences into the host chromosome. Cells which have stably integrated the introduced DNA into their chromosomes may be selected by also introducing one or more markers which allow selection of host cells which contain the expression vector. The marker may provide for prototropy to an auxotrophic host, biocide resistance, e.g., antibiotics, or resistance to heavy metals, such as copper or the like. The selectable marker gene can either be directly linked to the DNA sequences to be expressed, or introduced into the same cell by co-transformation. Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol. 3:280 (1983), and others.

Prokaryotic vectors used to express the proteins of the present invention include plasmids such as those capable of replication in E. coli such as, for example, PBR322, ColE1, pSC101, pACYC 184, πVX. Such plasmids are, for example, disclosed by Maniatis, T., et al. (In: Molecular Cloning, A Laboratory Manual, Cold Spring Harbor Press, Cold Spring Harbor, N.Y. (1982)). Bacillus plasmids include pC194, pC22 1, pT127, etc. Such plasmids are disclosed by Gryczan, T. (In: The Molecular Biology of the Bacilli, Academic Press, NY (1982), pp. 307-329). Suitable Streptomyces plasmids include pIJ101 (Kendall et al., J. Bacteriol. 169:4177-4183 (1987)), and Streptomyces bacteriophages such as øC31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704 (1986)), and Izaki, K. (Jpn. J. Bacteriol. 33:729-742 (1978)).

Once the vector or DNA sequence containing the constructs has been prepared for expression, the DNA constructs may be introduced or transformed into an appropriate host. Various techniques may be employed, such as protoplast fusion, calcium phosphate precipitation, electroporation, or other conventional techniques. As is well known, viral sequences containing the “hybrid” protein gene may be directly transformed into a susceptible host or first packaged into a viral particle and then introduced into a susceptible host by infection. After the cells have been transformed with the recombinant DNA (or RNA) molecule, or the virus or its genetic sequence is introduced into a susceptible host, the cells are grown in media and screened for appropriate activities. Expression of the sequence results in the production of the protein of the present invention.

Using antibodies specific for FGF-1 or Syn-1, co-immunoprecipitation of recombinant FGF-1 Cu²⁺-induced homodimer (see, e.g., Engleka et al., 1992 J. Biol. Chem. 267:11307) with recombinant p29 Syn-1 followed by FGF-1/Syn-1 immunoblot analysis may be used to confirm the interaction between FGF-1 and/or FGF-1 homodimer and p29 Syn-1. Varying the immunoprecipitation conditions may further permit determination of regulation FGF-1:Syn-1 interactions as a function of pS concentration, temperature, pH and ionic strength. Even if post-translational modifications of FGF-1/Syn-1 occur, sequencing of p29 Syn-1 will identify those residues that are post-translationally modified.

The p29 Syn-1 fragment, or any of the above-described fragments or genes may be expressed using known expression system, for example the Baculovirus, whereupon the p29 Syn-1 fragment would be expressed as a GST fusion protein. Fluorescence temperature spectroscopy studies of the FGF-1/p29 Syn-1 complex may also provide useful information relative to changes in protein conformation.

F. Regulation of FGF-1 Export

1. Inhibitors/Enhancers

A variety of useful inhibitors and enhancers that inhibit or promote the export of FGF-1 are contemplated by the present invention. As one will observe from a review of FIG. 1 and the related Examples, a variety of compositions and constructs are useful in this regard. Inhibitors/enhancers may act to regulate FGF-1 export by a variety of mechanisms. Regardless of the mechanism, inhibitors/enhancers inhibit or promote, respectively, FGF-1 export where such an effect can be readily determined according to the disclosure herein.

Without wishing to be bound by theory, inhibitors or enhancers may operate at the level of one or more of: (i) inhibition or potentiation of the activity of Syn-1-CE, the protease that cleaves Syn-1; (ii) an inhibitor/enhancer that is a regulatory molecule comprising a Syn-1 nucleic acid construct encoding a variant Syn-1 that is either protease resistant or highly protease sensitive, and in particular a Syn-1-CE resistant (or sensitive) Syn-1 variant; (iii) an inhibitor/enhancer that inhibits or promotes the binding of an FGF-1 export complex to a cytoplasmically accessible plasma membrane component of the inner leaflet of the plasma membrane (for example, pS), which may be accomplished by one or more of blocking (or promoting interaction with) inner leaflet binding sites with an inhibitor/enhancer molecule that specifically binds to such binding sites on the membrane, blocking (or promoting interaction with) membrane binding sites on Syn-1, blocking (or promoting interaction with) membrane binding sites on FGF-1, blocking (or promoting interaction with) membrane binding sites on S100A13, or blocking (or promoting interaction with) binding sites on any other FGF-1 export complex component or combinatorial membrane binding site formed by interaction of two or more such complex components; (iv) an inhibitor that is an antisense Syn-1 molecule or a ribozyme that specifically cleaves Syn-1 encoding mRNA; (v) an inhibitor/enhancer that blocks (or promotes) FGF-1 binding to Syn-1; or (vi) an inhibitor/enhancer that blocks (or promotes) S100A13 binding to FGF-1 export complexes, including binding to FGF-1, Syn-1 or to other FGF-1 complex components.

Description of these and related embodiments is presented below, in most cases with reference to inhibitors. Because, however, it is within the contemplation of the invention that under particular circumstances enhancement rather than inhibition of a particular intermolecular interaction within the FGF-1 export pathway for leaderless proteins may be desirable, it is intended that methods of regulating FGF-1 export from a cell include methods that inhibit FGF-1 export as well as methods that promote FGF-1 export, and the scope of the invention should not be otherwise limited.

a) Enzyme Regulatory Molecules

One “class” of inhibitors includes compounds or compositions that associate with an enzyme, such as a protease, in such a manner as to inhibit the normal function of the enzyme. Such inhibition can be effected by a variety of ways, including binding of the inhibitor to a site on the enzyme such that the substrate binding site is blocked through stearic hindrance; binding of the inhibitor to the active site of the enzyme and thus preventing the substrate from having access to the active site; binding of the inhibitor to the enzyme in such a manner that changes the secondary or tertiary structure of the enzyme and therefore inhibits its activity (allosteric effects); disrupting or preventing the interaction of a required cofactor with the enzyme; and in other ways appreciated by those of skill in the art.

b) FGF-1 Complex Assembly Regulatory Molecules

Another “class” of inhibitors includes compounds or compositions that prevent the interaction of one of the molecules in the FGF-1 export pathway with another molecule or with an intracellular vesicle (or component thereof), thereby disrupting the FGF-1 export pathway. For example, inhibitors that inhibit the formation of a complex comprising FGF-1, or inhibit the complexation of FGF-1 and Syn-1, are useful as described herein. Such inhibitors include compounds or compositions that bind to FGF-1, to Syn-1, to pS, or to any of the proteins in the FGF-1 export pathway, and prevent said protein(s) from interactions that are a prerequisite to the export of FGF-1. For example, an antibody (or immunologically active fragments thereof) which binds to Syn-1 in the cytosol, thereby making it unavailable for complex formation with FGF-1, are also useful as disclosed herein.

Antibodies may be polyclonal or monoclonal. In certain preferred embodiments, antibodies to be used as FGF-1 complex assembly regulatory molecules are monoclonal antibodies, and in more preferred embodiments immunologically active antibody fragments are used. Immunologically active antibody fragments comprise portions of any immunoglobulin molecule that retain the ability to specifically bind to a cognate antigen. Antibodies or immunologically active antibody fragments may include, but need not be limited to, antibodies or active fragments that are naturally occurring, induced or genetically engineered immunoglobulins or active fragments derived therefrom, including single chain antibodies, chimeric immunoglobulin fusion proteins, hybrid antibodies, monomeric or multimeric or oligomeric antibodies, or other forms of antibodies with which those having skill in the art will be readily familiar. In some preferred embodiments, antibodies or immunologically active antibody fragments are human immunoglobulins or “humanized” immunoglobulins having antigen binding domains identified in xenogeneic species and genetically engineered into human immunoglobulin framework sequences (See, e.g., U.S. Pat. Nos. 5,585,089, 5,530,101, 5,693,761 and 5,693,762, and references cited therein, all of which are hereby incorporated by reference in their entireties.).

c) Intracellular Trafficking Regulatory Molecules

Inhibitors that prevent or disrupt intracellular trafficking of any component of the FGF-1 export pathway (see, e.g., FIG. 1) also include compounds and compositions which inhibit or otherwise disrupt the expression of one or more proteins involved in the FGF-1 export pathway. Therefore, inhibitory agents that selectively interfere with the transcription and/or translation of genes that express the proteins involved in the FGF-1 export pathway are useful as disclosed herein, as are inhibitors which disrupt post-translational processing and modification of said proteins. (See, e.g., Muesch et al., 1990 Trends in Bioch. Sci. 15:86; Rothman 1994 Science 372:55; Cleves, 1997 Curr. Biology 7:R318; Jungnickel et al., 1994 FEBS Lett. 346:73.)

Also, as disclosed herein, a number of proteins and related molecules (e.g., nucleotides, cofactors, etc.) are found in a cell that has been exposed to some type of stress. As described herein, stable complexes comprising FGF-1 and Syn-1 have been isolated and identified.

The involvement of other proteins in the formation of a complex comprising FGF-1 and Syn-1 is also evident, in vivo as well as in vitro. Some of these “other proteins” have also been found in complexes that include FGF-1 and Syn-1, as disclosed herein.

Tracing the putative pathway back from the eventual release of FGF-1 from the cell—which pathway is broadly represented by the diagram set forth in FIG. 1 (albeit understood that the invention is not limited to, or by, the putative pathway)—one can appreciate that inhibitors or enhancers affecting FGF-1 export or release from the cell can be effective at various points in the pathway. For instance, such inhibitors or enhancers—which are referred to collectively herein as “agents”—may disrupt or stimulate the release of FGF-1 from the cell membrane. Alternatively, such agents may act to disrupt or stimulate the protease or protease complex that catalyzes the release of FGF-1 from the FGF-1:Syn-1 complex.

Because cyclophilin B (CpB) is believed to interact with Syn-1, and since CpB is present in various complexes which also include FGF-1 and Syn-1, agents which stimulate or inhibit the interaction between CpB and Syn-1 may also up-regulate or down-regulate FGF-1 release. Moreover, since the order of assembly of the involved molecules into the complex may be a critical factor in addition to—or separate from—the physical presence of the molecule in the complex, any agent which interferes with or streamlines the assembly process may be an effective inhibitor or enhancer, as well.

Thus, any agent which interferes with the participation of CpB in the formation of a complex containing FGF-1 and/or Syn-1 is expected to have an up- or down-regulatory effect on FGF-1 export. Useful inhibitors would thus include molecules which bind or otherwise affect CpB availability, including the cyclosporins (particularly cyclosporin A), steroids, and other agents with anti-inflammatory effects. Molecules such as estrogen, hsp90, cyclophilin A, and p59 may also be useful agents as described herein, as these molecules may form complexes with, or otherwise interact with CpB in a manner to make CpB unavailable (or less readily available) to participate in complexes comprising FGF-1.

Altering the amounts, availability and activities of calcineurin A, pS, and/or calmodulin, and modulating the calcium levels (e.g., the availability of Ca²⁺ ions to bind with pS/calmodulin) in the cell are expected to affect the association of molecules involved in FGF-1 release, whether those molecules are present in the final complex from which FGF-1 is released, or whether they are participants in intermediate events. Therefore, changes in the levels and interaction of the foregoing molecules is expected to stimulate or inhibit formation of the FGF-1-containing complex.

As disclosed herein, S100A13 is also involved in the putative pathway leading to FGF-1 release from the cell. Thus, any agent which interferes with or enhances the assembly of complexes containing S100A13 is expected to inhibit or stimulate FGF-1 export. Similarly, agents which disrupt or enhance the interaction of S100A13 with precursors in the series of interactions leading to the formation of complexes containing FGF-1 and/or Syn-1 will be useful.

Finally, agents which disrupt or enhance the interaction of Syn-1 with the secretory vesicle, or with the ability of Syn-1 to complex with FGF-1 or any other molecule involved in FGF-1-containing complexes, would be useful for the purposes disclosed herein. Logically, any agent which disrupts or stabilizes the conformation of any molecule involved in the FGF-1-containing complex would have an inhibitory or stimulatory effect, respectively.

2. Assays for Screening Inhibitors/Enhancers

Inhibitors of the secretion or export of leaderless proteins—and FGF-1 is described herein as exemplary, particularly within the context of the present invention—may be identified by an assay, such as the assays described herein. Briefly, in a preferred assay, a cell expressing FGF-1 or another leaderless protein (e.g. an FGF-1 mutein or a biologically active fragment thereof) is treated with the candidate inhibitor and the amount of FGF-1 detected as an extracellular protein is compared to the amount detected without treatment.

In any of the following assays (which are intended to be exemplary and do not constitute an exhaustive list), a compound inhibits export if there is a statistically significant reduction in the amount of protein detected extracellularly in the assay performed with the inhibitor compared to the assay performed without the inhibitor. Preferably, the inhibitor reduces export of the protein of interest by at least 50%, even more preferably 80% or greater, and also preferably, in a dose-dependent manner. In addition, there should be no statistically significant effect on the appearance of either the secreted protein or the cytosolic protein. Preferably, there is less than a 10% increase or decrease in the appearance of these two proteins.

Similarly, in any of these assays, a compound stimulates or enhances export if there is a statistically significant increase in the amount of protein detected extracellularly in the assay performed with the enhancer compared to the assay performed without the enhancer. Preferably, the enhancer increases export of the protein of interest by at least 50%, even more preferably 80% or greater, and also preferably, in a dose-dependent manner. In addition, there should be no statistically significant effect on the appearance of either the secreted protein or the cytosolic protein. Preferably, there is less than a 10% increase or decrease in the appearance of these two proteins.

Within the context of the present invention, therefore, an inhibitor must satisfy various criteria. First, it must inhibit the export of FGF-1. Second, it must not inhibit export of a representative secreted protein with a leader sequence. Third, it must not promote expression of a representative cytosolic protein in the extracellular environment. Once inhibitors satisfying these requirements are identified, the utilization of assay procedures to identify the manner in which the export of FGF-1 is inhibited are particularly useful.

Thus, inhibitors of FGF-1 export include various substances (e.g. therapeutic agents) which interfere with, inhibit or prevent (a) the formation of an FGF-1:Syn-1 complex; (b) the formation of a complex comprising (or including) FGF-1, Syn-1, or both; (c) the formation of a complex comprising FGF-1, Syn-1, cyclophilin-B, or any combination thereof; and (d) the formation of a complex comprising FGF-1, Syn-1, cyclophilin-B, S100A13, or any combination thereof. Inhibitors of FGF-1 export also include therapeutic agents which interfere with, inhibit or prevent (e) the release of FGF-1 from a complex including any one or more of the components recited in (a)-(d) above; or (f) trafficking of a complex comprising any one or more of the components recited in (a)-(d) above to the cell membrane.

Inhibitors of FGF-1 export contemplated within the scope of the present invention also include therapeutic agents which inhibit the trafficking of FGF-1 to the cell membrane by interfering with other mechanisms.

As disclosed herein, the present invention also contemplates the identification and use of therapeutic agents which stimulate or enhance the secretion of FGF—and more particularly, FGF-1. Thus, in a preferred assay, a cell expressing FGF-1 or another leaderless protein (e.g. an FGF-1 mutein or a biologically active fragment thereof) is treated with the candidate enhancing or up-regulating agent and the amount of FGF-1 detected as an extracellular protein is compared to the amount detected without treatment.

An enhancer must satisfy various criteria, within the context of the present invention. First, it must enhance the export of FGF-1. Second, it must not enhance export of a representative secreted protein with a leader sequence. Third, it must not promote expression of a representative cytosolic protein in the extracellular environment. Once enhancers satisfying these requirements are identified, the utilization of assay procedures to identify the manner in which the export of FGF-1 is enhanced are particularly useful.

Thus, enhancers of FGF-1 export include various substances (e.g. therapeutic agents) which stimulate or enhance (a) the formation of an FGF-1:Syn-1 complex; (b) the formation of a complex comprising (or including) FGF-1, Syn-1, or both; (c) the formation of a complex comprising FGF-1, Syn-1, cyclophilin-B, or any combination thereof; and (d) the formation of a complex comprising FGF-1, Syn-1, cyclophilin-B, S100A13, or any combination thereof. Enhancers of FGF-1 export also include therapeutic agents which enhance (e) the release of FGF-1 from a complex including any one or more of the components recited in (a)-(d) above; or (f) trafficking of a complex comprising any one or more of the components recited in (a)-(d) above to the cell membrane.

Enhancers of FGF-1 export contemplated within the scope of the present invention also include therapeutic agents which stimulate or increase the trafficking of FGF-1 to the cell membrane by enhancing or up-regulating other mechanisms.

Assays to detect leaderless protein, secreted protein, and cytosolic protein in a cell-based assay include immunoprecipitation of proteins labeled in a pulse-chase procedure, ELISA, 2-D gels, protein stains (e.g., Coomassie blue), HPLC, Western Blot, biological assays, and phagokinetic tracts. Such assays may be employed in the identification of useful up- or down-regulating therapeutic agents. In all these assays, test cells expressing and exporting a leaderless protein (e.g., FGF-1) or other protein of interest are incubated with and without the candidate inhibitor or enhancer.

Immunoprecipitation is an assay that may be used to determine inhibition or enhancement of the release/production of FGF-1 or any other protein(s) involved in the aforementioned complexes. Briefly, using FGF-1 as an example, cells that naturally express FGF-1 (or a biologically active fragment or mutein thereof) or express FGF-1 or a variant thereof from an introduced vector construct are labeled with ³⁵S-methionine and/or ³⁵S-cysteine for a brief period of time, typically 15 minutes or longer, in methionine- and/or cysteine-free cell culture medium. Following pulse-labeling, cells are washed with medium supplemented with excess unlabeled methionine and cysteine plus heparin if the leaderless protein is heparin-binding. Cells are then cultured in the same chase medium for various periods of time.

Candidate inhibitors or enhancers are added to cultures at various concentration. Culture supernatant is collected and clarified. Supematants are incubated with anti-FGF-1 immune serum or a monoclonal antibody, or with anti-tag antibody if a peptide tag is present, followed by a developing reagent such as Staphylococcus aureus Cowan strain I, protein A-Sepharose®, or Gamma-bind™ G-Sepharose®. (See, e.g., U.S. Pat. No. 5,437,995, which discloses an FGF-1 antibody useful as described herein. The disclosures of said patent are incorporated herein by reference.)

Immune complexes are pelleted by centrifugation, washed in a buffer containing 1% NP-40 and 0.5% deoxycholate, EGTA, PMSF, aprotinin, leupeptin, and pepstatin. Precipitates are then washed in a buffer containing sodium phosphate pH 7.2, deoxycholate, NP-40, and SDS. Immune complexes are eluted into an SDS gel sample buffer and separated by SDS-PAGE. The gel is processed for fluorography, dried, and exposed to x-ray film.

Alternatively, ELISA may be used to detect and quantify the amount of FGF-1 (or other protein(s) involved in the complexes noted herein) present in cell supernatants. ELISA is preferred for detection in high throughput screening. Briefly, when FGF-1 is the “test protein,” 96-well plates are coated with an FGF-1 antibody or anti-tag antibody, washed, and blocked with 2% BSA. Cell supernatant is then added to the wells. Following incubation and washing, a second antibody (e.g., to FGF-1) is added. The second antibody may be coupled to a label or detecting reagent, such as an enzyme or to biotin.

Following further incubation, a developing reagent is added and the amount of FGF-1 determined using an ELISA plate reader. The developing reagent is a substrate for the enzyme coupled to the second antibody (typically an anti-isotype antibody) or for the enzyme coupled to streptavidin. Suitable enzymes are well known in the art and include horseradish peroxidase, which acts upon a substrate (e.g., ABTS) resulting in a colorimetric reaction. It will be appreciated that rather than using a second antibody coupled to an enzyme, the FGF-1 antibody may be directly coupled to the horseradish peroxidase, or other equivalent detection reagent. If necessary, cell supernatants may be concentrated to raise the detection level.

ELISA may also be readily adapted for screening multiple candidate inhibitors or enhancers with high throughput. Briefly, such an assay is conveniently cell-based and performed in 96-well plates. If test cells naturally or stably express the protein of interest—e.g., FGF-1—the cells are plated at 20,000 cells/well. If the cells do not naturally express the protein, transient expression is achieved, such as by electroporation or Ca₂PO₄-mediated transfection.

For electroporation, an exemplary assay would comprise dispensing 100 μl of a mixture of cells (150,000 cells/ml) and vector DNA (5 μg/ml) into individual wells of a 96-well plate. The cells are electroporated using an apparatus with a 96-well electrode (e.g., ECM 600 Electroporation System, BTX, Genetronics, Inc.). Optimal conditions for electroporation are experimentally determined for the particular host cell type. Voltage, resistance, and pulse length are the typical parameters varied. Guidelines for optimizing electroporation may be obtained from manufacturers or found in protocol manuals, such as Current Protocols in Molecular Biology (Ausubel et al. (ed.), Wiley Interscience, 1987).

Cells are diluted with an equal volume of medium and incubated for 48 hours. Electroporation may be performed on various cell types, including mammalian cells, yeast cells, bacteria, and the like. Following incubation, medium with or without inhibitor or enhancer is added and cells are further incubated for 1-2 days. At this time, culture medium is harvested and the protein is assayed by any of the assays herein. Preferably, ELISA is used to detect the protein. An initial concentration of 50 μM is tested. If this amount gives a statistically significant reduction or increase of export, the candidate inhibitor or enhancer is further tested in a dose response.

Alternatively, concentrated supernatant may be electrophoresed on an SDS-PAGE gel and transferred to a solid support, such as nylon or nitrocellulose. The protein of interest is then detected by an immunoblot (see, e.g., Harlow, Antibodies: A Laboratory Manual, Cold Spring Harbor Laboratory, 1988), using an antibody to the protein of interest containing an isotopic or non-isotopic reporter group. These reporter groups include, but are not limited to enzymes, cofactors, dyes, radioisotopes, luminescent molecules, fluorescent molecules and biotin. Preferably, the reporter group is ¹²⁵I or horseradish peroxidase, which may be detected by incubation with 2,2′-azino-di-3-ethylbenzthiazoline sulfonic acid. These detection assays described above are readily adapted for use if the protein of interest contains a peptide tag. In such case, the antibody used will bind to the peptide tag. Other assays include size or charge-based chromatography, including HPLC, and affinity chromatography.

Other proteins of interest (e.g., FGF-2, PD-ECGF) may be assessed in similar manner. Proteins of interest displaying other functions may be assayed in other appropriate bioassays available in the art.

Alternatively, or as a further assessment of candidate inhibitors, inhibition assays may be performed by assaying the extent of binding between a protein of interest and its transport protein or proteins. A host cell expressing both the protein of interest and transport protein endogenously or following transfection are treated with candidate inhibitors. The binding may be measured by a variety of different methods. A co-precipitation assay in which antibodies to either protein are used to precipitate the preselected proteins may be used. The precipitates are assayed by gel electrophoresis for disruption of the interaction. The protein of interest may be a fusion protein with a tag peptide and anti-tag peptide antibodies are used for precipitation. As described above, the membrane bound α1 subunit or fragment may be used in this assay.

An assay for identifying an inhibitor or enhancer of export may be performed using isolated transport molecule(s) and the protein of interest. Isolated components are preferably obtained by recombinant expression and purified by standard methodologies. In such an assay, the isolated components are mixed, along with any necessary cofactors, in the presence or absence of the candidate inhibitor or enhancer. The extent of binding of the protein of interest and transport molecule is measured. This assay may conveniently be performed in an ELISA or ELISA-style format.

Briefly, using inhibitors as exemplary, the transport molecule is adhered to the wells of a 96-well plate. The protein of interest with or without candidate inhibitors is added to the wells and incubated. Unbound protein is washed away, and the protein of interest is detected by labeled antibody as described herein, for example. Variations on this assay may be used. For example, the components may be attached to Biacore™ chips (Biacore AB, Uppsala, Sweden) or similar solid phase detection device.

The activity of a therapeutic agent of interest (e.g., inhibitor or enhancer) may also be measured by in vivo models of disease. For example, a cell that exports the protein of interest to the extracellular environment is introduced into a local milieu where the activity of the protein can be measured. In the case of a cell that exports FGF-1, for example, the cell will promote vascularization or angiogenesis, inflammation, clotting, or gliosis on neighboring cells. For example, in the case of a cell exporting FGF-1 that is inoculated along with tumor cells, vascularization of the tumor will ensue. Accordingly, an inhibitor of FGF-1 export will inhibit growth of the tumor. One skilled in the art will recognize that the export levels of the protein may be varied through the use of promoters of varying strength. In addition, cells exporting the protein may be transformed stably or express the protein transiently. The site and route of administration depends in part upon the protein and its normal site of action.

For proteins of interest that cause cell motility, such s FGF, a phagokinetic tract assay may be used to determine the amount of protein of interest exported (Mignatti et al., J. Cellular Physiol. 151:81-93, 1992). In this assay, cells are allowed to migrate on a microscope cover slip coated with colloidal gold. Under dark field illumination, the gold particles appear as a homogenous layer of highly refringent particles on a dark background. When a cell migrates on the substrate, it pushes aside the gold particles producing a dark track. An image analyzer may be used to measure the length of the tracks. Under predetermined, standard conditions, cell motility directly correlates with the amount of FGF-2 produced by the cells. The choice of the bioassay will depend, at least in part, by the protein of interest tested.

In vivo assays may be used to confirm that an inhibitor or enhancer affects export of protein of interest. For measuring angiogenic activity, standard assays include the chicken chorioallantoic membrane assay (Aurbach et al., Dev. Biol. 41:391, 1974; Taylor and Folkman, Nature 247:307, 1982) and inhibition of angiogenesis in tumors. For some proteins of interest, an assay measuring inhibition of tumor growth, such as in a murine xenogeneic tumor model, may be appropriate.

3. Antisense Regulation of FGF-1 Export

Nucleic acids and oligonucleotides for use as described herein can be synthesized by any method known to those of skill in this art (see, e.g., WO 93/01286, U.S. application Ser. No. 07/723,454; U.S. Pat. No. 5,218,088; U.S. Pat. No. 5,175,269; U.S. Pat. No. 5,109,124). Identification of oligonucleotides and ribozymes for use as antisense agents and DNA encoding genes for targeted delivery for genetic therapy involve methods well known in the art. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)).

Antisense nucleotides, triplex molecules, and ribozymes may also be useful regulators of protein export, for example as inhibitors or enhancers of FGF-1 export. Antisense nucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary to or bind the sense strand of DNA or mRNA that encodes a protein involved in an FGF-1 export pathway, or a protein mediating any other unwanted process such that inhibition of translation of the protein is desirable.

A ribozyme is an RNA molecule that specifically cleaves RNA substrates, such as mRNA, resulting in inhibition or interference with cell growth or expression. There are at least five known classes of ribozymes involved in the cleavage and/or ligation of RNA chains. Ribozymes can be targeted to any RNA transcript and can catalytically cleave such transcript (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). Any such ribozyme or nucleic acid encoding the ribozyme may be delivered to cells as disclosed herein or by methods with which those skilled in the art will be familiar.

Ribozymes, and the like may be delivered to the targeted cells by DNA encoding the ribozyme linked to a eukaryotic promoter, such as a eukaryotic viral promoter, such that upon introduction into the nucleus, the ribozyme will be directly transcribed. In such instances, the construct will also include a nuclear translocation sequence, generally as part of the ligand or as part of a linker between the ligand and nucleic acid binding domain.

As non-limiting examples according to the present invention, antisense and/or ribozyme constructs may be targeted to alter the expression in cells of Syn-1 and/or any other component of the FGF-1 export pathway. Such regulation may be applicable inhibition or promotion of FGF-1 export, to regulation of clathrin associated receptor recycling or cellular responses to extracellular ligands that interact with cell surface receptors, or to other regulated processes.

G. Administration

As described above, FGF-1 is useful for repairing wounded, injured and/or damaged tissue or organs among other uses. Repair or treatment of means that symptoms are lessened, or the progression of the disease or conditions are halted or delayed, or wounded, injured or damaged tissue or organs are made measurably or noticeably less wounded, injured or damaged as determined by at least one criterion, or healed. Therefore, agents useful for the enhancement or stimulation of secretion and/or export of FGF-1 into the bloodstream are advantageous for accelerating, repairing or healing wounded, injured and or damaged tissue.

In contrast, the over expression of FGF-1 has been associated with the uncontrolled production of tissue, such as occurs in the initial development or growth of tumors, including cancerous tumors. Consequently, agents useful for inhibiting, reducing or abolishing the secretion and/or export of FGF-1 into the bloodstream are advantageous for inhibiting, reducing, or halting the growth of a particular class of unwanted tissue, including tumors.

By example, cells to be treated are contacted with an inhibitor or an enhancer at a therapeutically effective dosage. Contacting may be effected by incubation of cells ex vivo or in vivo, such as by topical treatment, delivery by specific carrier, or by vascular supply.

Similarly, therapeutic agents which up-regulate, stimulate or otherwise facilitate the secretion of FGF-1 are also useful for ameliorating a variety of conditions, including repair situations, such as the repair of spinal cord, nerve or ulcerative damage, injury or degeneration.

The inhibitors or enhancers disclosed herein may be formulated into pharmaceutical compositions suitable for topical, local, intravenous and systemic application. Time release formulations are also desirable. Effective concentrations of one or more of the conjugates are mixed with a suitable pharmaceutical carrier or vehicle. The concentrations or amounts of the conjugates that are effective requires delivery of an amount, upon administration, that ameliorates the symptoms or treats the disease. Typically, the compositions are formulated for single dosage administration.

Therapeutically effective concentrations and amounts may be determined empirically by testing the therapeutic agents or substances of the present invention in known in vitro and in vivo systems, such as those described herein; dosages for humans or other animals may then be extrapolated therefrom.

Candidate tumors for treatment as described herein include those with receptors for FGF. Such tumors include, but are not limited to, melanomas, teratocarcinomas, ovarian carcinomas, bladder tumors, and neuroblastomas. Other diseases, disorders, and syndromes are suitable for treatment and include rheumatoid arthritis, diabetic retinopathy, psoriasis, and the like.

Pharmaceutical carriers or vehicles suitable for administration of the therapeutic agents provided herein include any such carriers known to those skilled in the art to be suitable for the particular mode of administration. In addition, the agents may be formulated as the sole pharmaceutically active ingredient in the composition or may be combined with other active ingredients.

The compositions of the present invention may be prepared for administration by a variety of different routes. Local administration is preferred. The therapeutic agent may be mixed with suitable excipients, such as salts, buffers, stabilizers, and the like. If applied topically, such as to the skin and mucous membranes, the agent may be in the form of gels, creams, and lotions. Such solutions, particularly those intended for ophthalmic use, may be formulated as, for example, 0.01%-10% isotonic solutions, pH about 5-7, with appropriate salts (see, e.g., U.S. Pat. No. 5,116,868).

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of toxicity such as sodium chloride or dextrose. Parenteral preparations can be enclosed in ampules, disposable syringes or multiple dose vials made of glass, plastic or other suitable material.

If therapeutic agents of the present invention are administered intravenously, suitable carriers include physiological saline or phosphate buffered saline (PBS), and solutions containing thickening and solubilizing agents, such as glucose, polyethylene glycol, and polypropylene glycol and mixtures thereof. Liposomal suspensions may also be suitable as pharmaceutically acceptable carriers. These may be prepared according to methods known to those skilled in the art.

The therapeutic agent may be prepared with carriers that protect it against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others. For example, the composition may be applied during surgery using a sponge, such as a commercially available surgical sponge (see, e.g., U.S. Pat. Nos. 3,956,044 and 4,045,238).

The agents or substances of the invention can be administered by any appropriate route, for example, orally, parenterally, intravenously, intradermally, subcutaneously, or topically, in liquid, semi-liquid or solid form and are formulated in a manner suitable for each route of administration. Preferred modes of administration depend upon the indication treated. Dermatological and ophthalmologic indications will typically be treated locally; whereas, tumors, restenosis, and infections will typically be treated by systemic, intradermal or intramuscular modes of administration.

The therapeutic agent is included in the pharmaceutically acceptable carrier in an amount sufficient to exert a therapeutically useful effect in the absence of undesirable side effects. It is understood that number and degree of side effects depends upon the condition for which the agents are administered. For example, certain toxic and undesirable side effects are tolerated when treating life-threatening illnesses, such as tumors, that would not be tolerated when treating disorders of lesser consequence. The concentration of therapeutic agent in the composition will depend on absorption, inactivation and excretion rates thereof, the dosage schedule, and amount administered, as well as other factors known to those of skill in the art.

The therapeutic agent may be administered one time, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

H. FGF Constructs.

Constructs expressing FGF alone or as a fusion protein are advantageous for use as described in the Examples that follow. The production of sufficient quantities of expression product (e.g., recombinant variants of FGF-1) for use in the various model systems and assays as disclosed herein advantageously employs a variety of known cell lines and vectors. Constructs expressing FGF as provided herein may include constructs encoding and expressing truncated FGF, as described above.

For example, U.S. Pat. No. 5,395,756 describes the production of acidic FGF protein (FGF-1), using an expression vector containing a cDNA sequence encoding a human acidic FGF protein with a T7 promoter upstream therefrom. Vectors including a sequence encoding a deletion-type mutein lacking up to 43 amino acids at the N-terminus are also disclosed. Accordingly, vectors useful in the transformation of host cells are available in the art, as are methods for transfecting same to permit the resultant expression of the gene/gene product of interest.

There are also advantages to maintaining the Cys30 residue and to deleting the first 20 amino acid residues. Cys30, in addition to participating in FGF-1 dimerization as described above, enables FGF-1 to subsequently utilize its COOH-terminal domain (residues 112-138) to associate with pS in an unfolded state at 42° C. Because the ps-binding domain in FGF-1 coincides with the domain responsible for cytosolic retention of the intracellular FGF-1 translation product as a non-nuclear, cytosolic protein, FGF-1:β-gal mutants may be used to define the boundaries of this domain required for FGF-1₁₋₁₅₄ secretion.

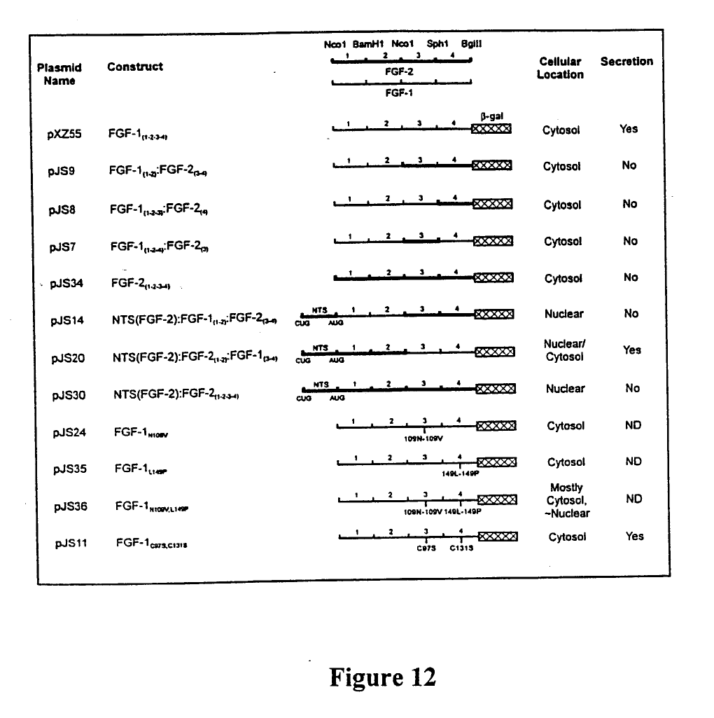

A number of synthetic FGF-1 cDNAs are available to construct a variety of FGF-1:FGF-2 cassette mutants, each containing the reporter gene, β-gal and stable NIH 3T3 cells. A list of exemplary chimera and point mutant FGF-1:β-gal chimera, each of which may be obtained, is shown in FIG. 12. The intracellular traffic of these chimeric proteins has been studied in detail using confocal β-gal immunofluorescence, X-gal staining and β-gal immunoblot analysis of cytosol and nuclear fractions prepared as previously described (see, Zhan et al. (1992)). These studies also suggest that the domain in FGF-1 responsible for cytosolic retention which enables FGF-1 to access the FGF-1 export pathway is not present in FGF-2 (see, FIG. 12). Moreover, FGF-2:β-gal is not exported in response to temperature stress (data not shown) under conditions identical to those described for the data in FIG. 12. These data also confirm the premise that nuclear-associated FGF-1 is not accessible to the FGF-1 export pathway.

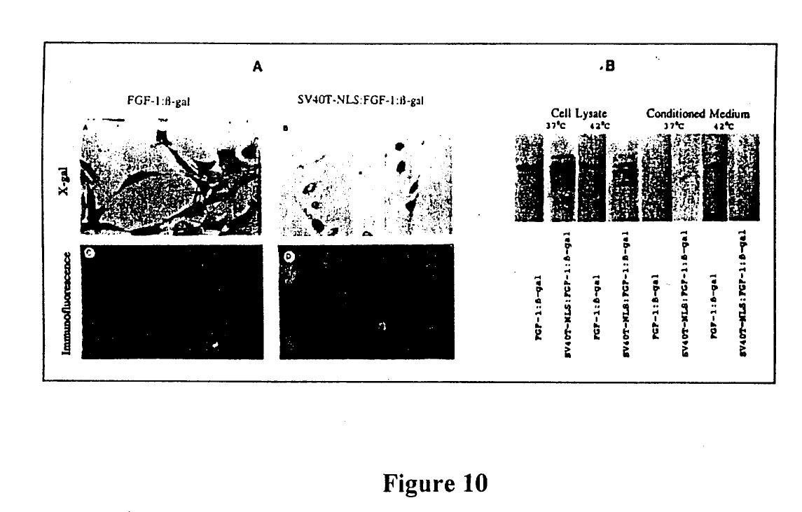

FIG. 12 shows FGF-1 mutants and chimeric constructs prepared by recombinant circle PCR (RC-PCR; see Friedman, et al., Biochem. Biophys. Res. Commun. 198: 1203-8 (1994)). Stable 3T3 cell transfectants for each of these constructs have been isolated, and the cytosolic level of the individual FGF-I₂₁₋₁₅₄ mutant/chimeric proteins are similar as measured by FGF-1 immunoblot analysis. The traffic of the individual constructs are shown; these data are derived from cell fractionation/immunoblot analysis, X-gal staining and β-gal immunohistochemical analysis as described in FIG. 10. Export (mislabeled on the drawing as “secretion”) of the individual construct was measured as described in FIG. 10. NTS=Nuclear Translocation Signal.

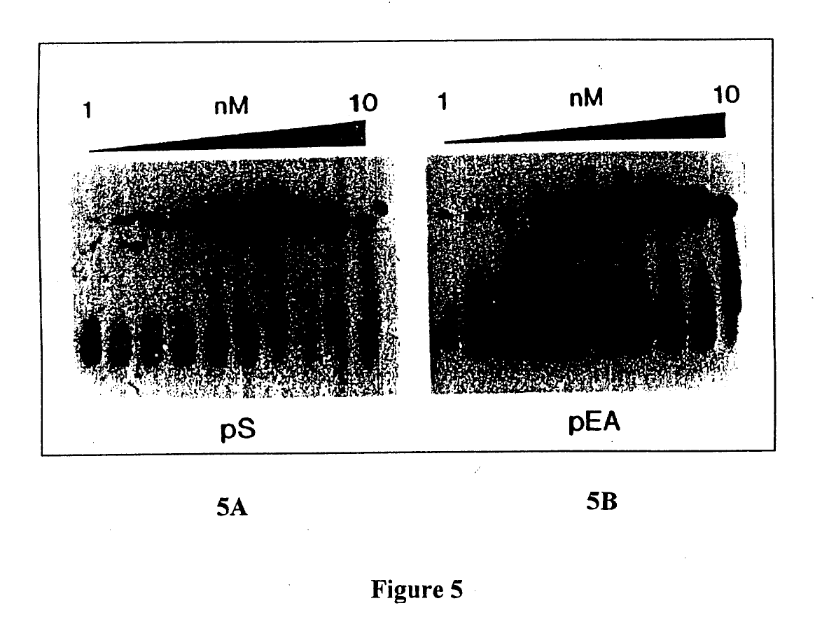

Of particular interest is the secretion of the N109 point mutant (FIG. 12), which exhibits cytosolic localization. This mutant is of interest because the FGF-1 crystalline structure (Zhu et al., Science 251:90-93 (1991)) predicts that residue N109 may be involved in the formation of a hydrogen bond with residue K124. Thus, if a mutant such as the FGF-1 N109:β-gal mutant is not exported in response to temperature stress as proposed, the residue can be mutated in fill length FGF-1₁₋₁₅₄:β-gal (I) to determine the cytosol/nuclear locale of the chimera using the immunoblot/cell fractionation, X-gal staining and confocal immunofluorescence microscopy strategies as described (Zhan et al. (1992); Savion et al., (1994)); (ii) to determine whether the chimera is accessible to the heat shock-induced FGF-1 export pathway using conventional FGF-1 and/or β-gal immunoblot analysis of (NH₄)₂SO₄ and DTT activated conditioned medium as described (Jackson et al. (1992); Jackson et al. (1995); Tarantini et al. (1995)); and (iii) to determine not only whether the N109 point mutant is biologically active as a mitogen using established biological growth and DNA synthesis assays (e.g., Maciag et al. 1981 and 1984), but also whether the N109 point mutant is able to bind specifically to pS using the solid phase-binding (Tarantini et al. (1995)) and native gel shift assays as described in Example 3 and FIG. 5.

For example, wild-type FGF-1₁₋₁₅₄ translation is relatively poor in both conventional prokaryotic and eukaryotic expression systems. This poor translational efficiency of FGF-1 may be related to the presence of an exaggerated stem-loop structure with the FGF-1₁₋₁₅₄ mRNA, which was predicted by computer modeling (Forough et al., Biochem. Biophys. Acta 1090: 293-298 (1991)). The sequence of DNA encoding human FGF-1 has been reported in Jaye et al., Science 233:541-545, 1986; also see U.S. Pat. No. 5,223,483. It is also anticipated that muteins of FGF-1 will be useful in the within-disclosed compositions, complexes and according to the methods disclosed herein (see, e.g., U.S. Pat. No. 5,223,483).

Interestingly, mammalian cells are known to constitutively express reasonable levels of the FGF-1 transcript containing relatively small levels of the FGF-1 protein (Libermann et al., EMBO J. 6: 1627-1632 (1987)). However, prior to the present invention, it has been difficult to study FGF-1 export using these cell types in vitro.

In order to circumvent the apparent inefficient translation of the FGF-1₁₋₁₅₄ mRNA, a synthetic gene encoding FGF-1₂₁₋₁₅₄ was prepared, in which the putative stem-loop mRNA structures have been minimized, especially the exaggerated stem structure which may result from the hybridization of the 3′-and 5′-ends of the FGF-1₁₋₁₅₄ ORF (see, Forough et al., (1991)). All of the functional (heparin-binding, receptor-binding) and biological (induction of cell migration, growth, DNA synthesis, angiogenesis, and neurotrophic behavior) features in vitro and in vivo are conserved in FGF-1₂₁₋₁₅₄ and are indistinguishable from those observed with FGF-1₁₋₁₅₄ (Burgess et al., J. Biol. Chem. 260:11389-11392 (1985)).

Moreover, expression of FGF-1₂₁₋₁₅₄ with or without the FGF-4₍ₛₛ₎ leader from stable transfectants (using the pMEXneo expression vector) in mammalian cells yields sufficient functional protein which is readily visualized using conventional heparin adsorption and immunologic procedures. The FGF-4 signal or leader sequence (FGF-4₍ₛₛ₎ is useful in the production of soluble FGF-1—i.e., an FGF-1 molecule including FGF-4₍ₛₛ₎ is forcibly transported out of the cell. NIH 3T3 cells have proven to be useful in the production of FGF-1 molecules when transfected with FGF-1₂₁₋₁₅₄.

Other deletions of the FGF-1 gene may accomplish high expression of a functional FGF-1 protein, so long as regions encoding critical functional and biological features of the protein are conserved to retain the in vitro and in vivo function of the molecule. Key attributes of the molecule appear to include, for example, both the cys30 residue and the COOH-terminal domain (residues 112-138), as disclosed below in sections addressing the construction of the FGF-1 molecule.

In one embodiment of the invention, the FGF-1₁₋₁₅₄ N109 construct is expressed using the pET3c expression vector, and the recombinant protein purified using either heparin and/or Cu²⁺ Sepharose affinity and/or reversed phase and/or ion exchange HPLC methods. The FGF-1₁₋₁₅₄ N109 mutant will traffic to the cytosol, but will not be exported in response to heat shock and will associate poorly, if at all, with pS. Similar studies with FGF-1₁₋₁₅₄ K124:β-gal will confirm these data.

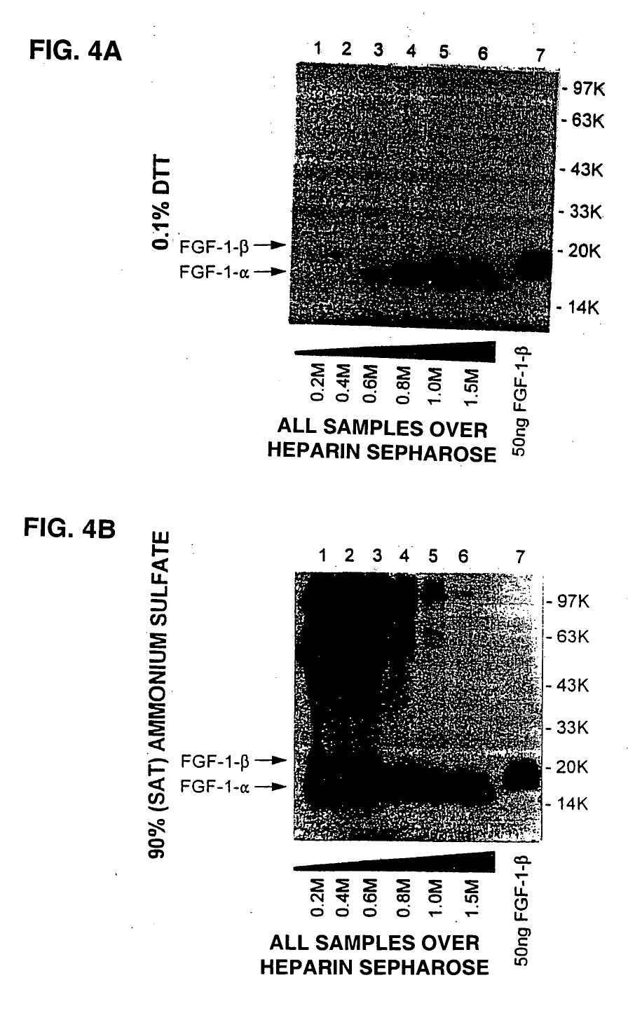

Another embodiment demonstrates that the model pathway established for FGF-1 export (FIG. 1), using FGF-1 lacking the NH₂-terminal residues 1-20 also applies to the full length FGF-1₁₋₁₅₄ translation product. The endogenous FGF-1₁₋₁₅₄ protein is observed in the conditioned medium in response to temperature stress (FIG. 4). Consequently, the FGF-1₂₁₋₁₅₄ construct was originally used so that it could be distinguished from the endogenous FGF-1₁₋₁₅₄ and the transfected FGF-1₂₁₋₁₅₄ proteins. Both forms of FGF-1 are indistinguishable in terms of their abilities to modify the cell migration and growth of a wide variety of target cells both in vitro and in vivo. Moreover, the expression of FGF-1₁₋₁₅₄ in the NIH 3T3 cell is only approximately 60% lower than the expression of the FGF-1₂₁₋₁₅₄ protein. Thus, secretion of FGF-1₁₋₁₅₄ should be reportable. However, even if it were not, an FGF-1₁₋₁₅₄:β-gal chimera could be readily constructed to study the secretion of the chimera after obtaining stable NIH 3T3 cell transfectants using increased sensitivity afforded by the enzymatic activity of the reporter gene as previously described (Zhan et al. (1992); Savion et al., (1994)).

Although these experiments have been exemplified in NIH 3T3 cells, the proposed mechanisms are expected to be applicable to other cell types. Thus, for example, endothelial cells (EC) and vascular smooth muscle cells (SMC) may be transfected with the FGF-1₁₋₁₅₄:β-gal chimera using the pMEXneo or other mammalian expression plasmid. Moreover, the exemplified use of the FGF-1₁₋₁₅₄:β-gal chimera permits the evaluation of the release of both the chimera and the endogenous form of FGF-1 since immunoblot analysis will resolve these proteins as Mᵣ 118 kDa and 20 kDa proteins.

As demonstrated in the NIH 3T3 cell, extracellular markers, such as lactate dehydrogenase, may be used to indicate cell lysis and to identify stable vascular cell transfectants expressing β-gal, IL-1α and FGF-2 for use as alternative controls. When stable β-gal, IL-1α and FGF-2 NIH 3T3 cell transfectants have been utilized, none of these proteins were released in response to temperature stress (data not shown). Thus, the model FGF-1 export pathway is generally applicable to numerous cell lines as well, including, but not limited to, HUVEC-ECV cells, murine LE-II cells, murine adipose EC and various rat SMCs. As those of skill in the relevant art will appreciate, there may be some differences between the various cell lines; however, such differences do not exceed the scope of the present invention.

As described in more detail herein, the levels of extracellular FGF-1 release in response to heat shock can be accurately quantitated by methods routinely used in the art, such as ELISAs. The stability of the recombinant FGF-1 protein, as well as the potential interference of endogenous fluids with the FGF-1 protein are readily determined prior to beginning the ELISA measurement for in vivo correlates. Control of other variables is within the routine practice of one of ordinary skill in the art.

In order that those skilled in the art can more fully understand this invention, the following examples are set forth. These examples are included solely for the purpose of illustration, and various modifications may be made without deviating from the spirit and scope of the invention. Accordingly, the invention is not limited except as by the appended claims.

### EXAMPLES

In the following examples and protocols, restriction enzymes, ligase, labels, and commercially available reagents were utilized in accordance with the manufacturer's recommendations. Standard methods and techniques for cloning, isolation, purification, labeling, and the like, as well as the preparation of standard reagents were performed essentially in accordance with Molecular Cloning: A Laboratory Manual, second edition, edited by Sambrook, Fritsch & Maniatis, Cold Spring Harbor Laboratory, 1989, and the revised third edition thereof.

Antibodies are essential to many of the following experiments. Rabbits are preferred for production of polyclonal antibodies. Procedurally, six rabbits are injected intradermally or intramuscularly with GST fusion or MAP peptides suspended in Freund's Complete Adjuvant (1:1), and bled in accordance with standard procedures. Booster injections are administered as necessary to maintain optimum antibody titer.

### Example 1

### Characterization of P40 SYN-1 as a Heparin- and Phosphatidylserine (PS)-Binding Protein

The serendipitous observations of Perin et al. (1991) led the present inventors to examine the conditioned medium of heat-shocked FGF-1 NIH 3T3 transfectants for the presence of Syn-1. Medium conditioned by heat shock (2 hr., 42° C.), as well as conditioned media (2 hr., 37° C.) was obtained from FGF-1 NIH 3T3 cell transfectants, activated by the addition of (NH₄)₂SO₄ and absorbed/eluted from heparin Sepharose. An immunoblot analysis of each sample was performed using a Syn-1 antibody. The antibodies to Syn-1 and to FGF-1 used and discussed throughout the present experiments were produced in the inventors' laboratories through the use of a combination of synthetic or recombinant Syn-1 and/or FGF-1 protein molecules. Recombinant p40 Syn-1 (100 ng in lane 3) served as a positive control.

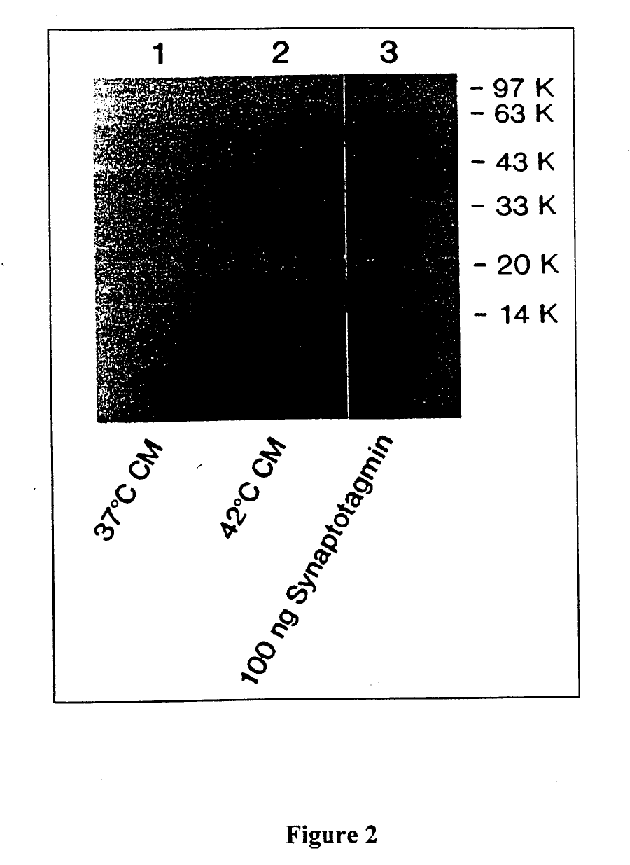

As shown in FIG. 2, by comparing lane 2 with the control media, a Syn-1 fragment was identified as a p40 heparin-binding protein. The mobility of the protein is interesting in that the recombinant p40 Syn-1 has an apparent Mᵣ 42 kDa, while the Syn-1 in the temperature conditioned media has an apparent Mᵣ ˜40 kDa.

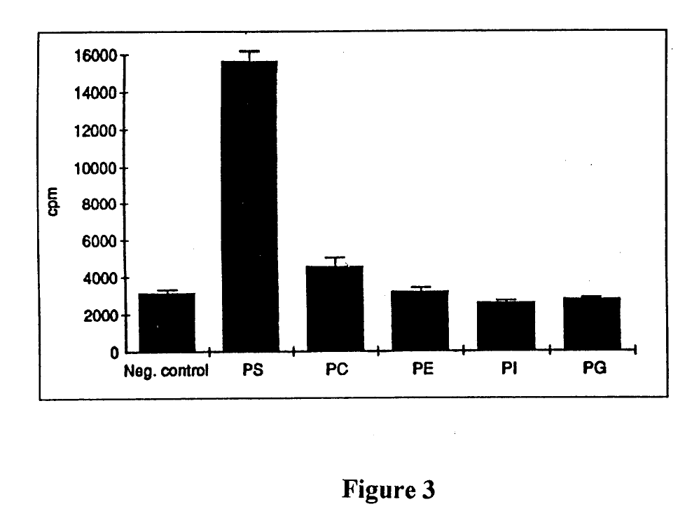

The cDNA encoding human Syn-1 (p65) was then used to express the p40 form of Syn-1 in the pGEX-KG prokaryotic expression system (Pharmacia) as a GST fusion protein. Recombinant human p40 Syn-1 was expressed in the pET3 expression system, purified by heparin affinity and RP-HPLC, iodinated using lactoperoxidase methods according to Engleka et al. (1992), and added to plastic wells coated with 10 μM phospholipids as described by Perin et al., Nature 345:260-263 (1990). Negative control, no phospholipid; pS, phosphatidylserine; pC, p1 choline; pE, pL ethanol-amine; p1, pL inositol; and pG, pL glycerol. Data reported as CPMs bound (±std deviation) after three washes with 0.1M Tris, pH 7.4 containing 0.15 M NaCl. As shown in FIG. 3, the pLS-binding property of p40 Syn-1 was confirmed.

Iodination of the recombinant p40 protein permitted the characterization of p40 Syn-1 as a heparin-binding protein which elutes from immobilized heparin at 0.6M NaCl (data not shown). Interestingly, the p40 protein, originally characterized in 1985 as a heparin-binding protein, eluted with FGF-1 at 1.0M NaCl. The apparent discrepancy in the property of heparin elution was explained as an interaction between p40 Syn-1 and FGF-1, or as a post-translational modification of p40 Syn-1.

### Example 2

### FGF-1 is Present in Temperature-Conditioned Medium as a High Mᵣ Complex

Although evidence points to interaction between p40 Syn-1 and FGF-1, to date conventional methods (gel analysis, exclusion chromatography and ligand affinity chromatography at a variety of pH and ionic strengths) have failed to provide evidence of direct interaction between these recombinant proteins. Consequently, experiments were designed to determine whether SDS was interfering with the resolution of FGF-1 as a large Mᵣ complex.

Immunoblot analysis of conditioned medium from heat shocked FGF-1 NIH 3T3 transfectants was activated by addition of either DTT or (NH₄)₂SO₄, absorbed to heparin Sepharose, and gradient eluted with NaCl. A non-reduced, limited SDS-Page FGF-1 immunoblot was performed using FGF-1 antiserum at pH 7.2 on the samples as described by Jackson et al. (1992). SDS was eliminated from the running gel.

As shown in FIG. 4, FGF-1 can be clearly identified as a single band with a characteristic heparin elution profile. However, although FGF-1 eluting from immobilized heparin near 1.0M NaCl was present as a single band, FGF-1 eluting from the heparin column prior to 0.6M NaCl were resolved as multiple bands. Because proteins may migrate in the limited SDS-PAGE gel on the basis of both size and charge, it is not possible to assign accurate Mᵣ to these bands. Hence Mᵣs have been assigned for comparative purposes. However, the results have been interpreted as indicating that high Mᵣ FGF-1-containing complexes can be identified by limited SDS-PAGE analysis of (NH₄)₂SO₄-activated, but not from DTT-activated heat-shocked conditioned medium from NIH 3T3 FGF-1 transfectants.

The sensitivity of the high Mᵣ FGF-1 containing complexes also demonstrates the significance of FGF-1 cysteine residues in complex formation. Purification of the FGF-1 present in the 0.2-0.4M NaCl-eluted fractions yielded FGF-1 as a homodimer.

### Example 3

### FGF-1 is a Phosphatidylserine (pS)-Binding Protein

An examination of the ability of FGF-1 to associate with phospholipids using conventional phospholipid-binding methods suggested that while FGF-1 is able to associate with pS, FGF-1 does not recognize other phospholipids (Lippincott-Schwartz et al. (1989)). It was possible to utilize synthetic peptides to map the domain within FGF-1 responsible for pS-binding, and the domain defined by residues 112-138, which has been previously characterized as the dominant heparin-binding domain in FGF-1 (Burgess et al., J. Cell Biol. 111:2129-2138 (1990)), and which appeared to be involved in the binding to pS (Tarantini et al., (1995)). In addition, the cys-free FGF-1 mutant which binds heparin and is active as a BALB/c 3T3 cell mitogen was not able to associate with pS. Id.

To confirm the FGF-1 pS-binding activity established by these solid phase binding assays, novel gel retardation methods were used to visualize the FGF-1:pS complex. Native PAGE (pH 6.8) analysis was performed with varying amounts of phosphatidylserine (pS) and phosphatidylethanolamine (pEA), but with a constant amount of [¹²⁵I]-FGF-1. The pL were premixed with cold FGF-1 (10 ng), followed by the addition of the [¹²⁵I]-FGF-1 probe, and the entire sample was subjected to electrophoresis with the exception of the last lane in each gel which contained 50 μg of either pS or pEA containing cold FGF-1 (10 ng) which was loaded without the probe. In this situation, [¹²⁵I]-FGF-1 was added to the top of the lane immediately prior to electrophoresis in an attempt to visualize the level of non-specific trapping of the probe in the pL micelle.

As shown in FIG. 5, the migration of FGF-1 in native PAGE gels can be significantly retarded by the presence of increasing concentrations of pS. In contrast, pEA was inefficient in retarding the electrophoretic mobility of FGF-1. The combined data indicates that FGF-1 is a pS-binding protein, suggesting that the interaction between Syn-1 and FGF-1 involves an interaction between the phospholipid component of these proteins as a co-factor, and that the interaction is involved in the regulation of FGF-1 export. Indeed, ligand affinity chromatography with immobilized FGF-1 and/or Syn-1 and conventional gel exclusion chromatography is used in the presence and absence of pS to monitor the formation of an FGF-1:Syn-1 complex as a means of re-evaluating the earlier results.

### Example 4

### The Role of Cu²⁻ Oxidation to Induce Heterodimer Formation Between FGF-1 and P40 Syn-1

In view of the following previously established principles: (I) reducing agents are able to activate the latent form of FGF-1 resolved in temperature-conditioned medium as an inefficient heparin-binding complex, (ii) both Syn-1 and FGF-1 are heparin-and pLS-binding proteins, (iii) Cu²⁺ is able to induce FGF-1 homodimer formation, and (iv) FGF-1 cys residues are critical for FGF-1 export, experiments were designed to examine the ability of Cu²⁺ to oxidized Syn-1 homodimer formation and Syn-1:FGF-1 heterodimer formation.

Purified FGF-1 and p40 Syn-1 were incubated with, and without, Cu²⁺ for 10 min. and resolved on two non-reduced SDS-PAGE gels (A and B), as described by Maciag et al., Recent Prog. Horm. Res. 49:105-123 (1993). The systems were identical except gel 5B was immunoblotted with FGF-1 antibody, whereas gel 5A was not.

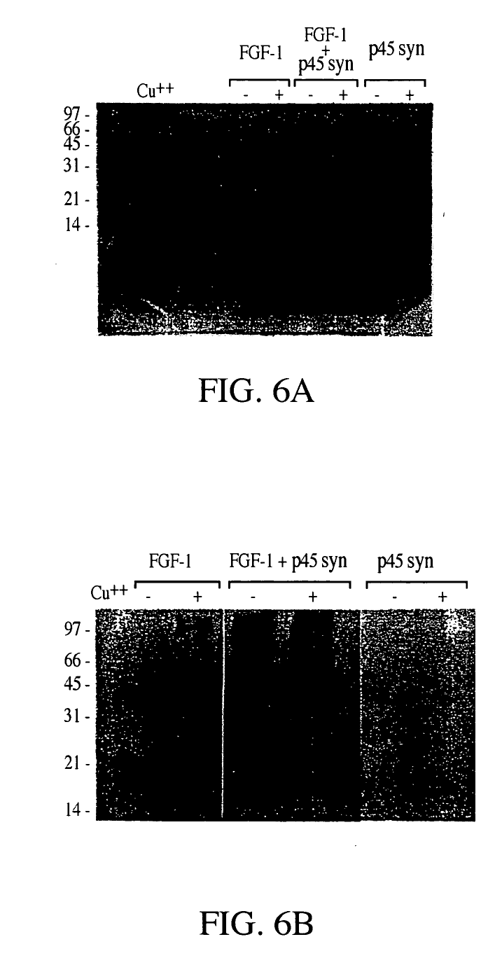

As shown in FIG. 6A, Cu²⁺ is able to induce the formation of the FGF-1 homodimer but was not able to induce the formation of a Syn-1 homodimer. However, when FGF-1 and p40 Syn-1 were added together, non-reduced SDS-PAGE was able to resolve a unique component with an apparent Mᵣ˜66,000 (FIG. 6A). To determine whether this band contained FGF-1, this experiment was repeated and analyzed by FGF-1 immunoblot analysis. As shown in FIG. 6B, the band previously resolved as a putative Syn-1:FGF-1 heterodimer contained FGF-1.

### Example 5

### Co-Localization of Syn-1 and FGF-1 as a High Mᵣ Complex in Temperature Conditioned Medium

In view of the following previously established principles: (I) FGF-1 and p40 Syn-1 are both pS-binding proteins, (ii) FGF-1 and Syn-1 may associate with each other through a pS:protein rather than in a direct protein:protein interaction, and (iii) FGF-1 can be resolved as a low ionic strength heparin-binding and DTT sensitive high Mᵣ complex in temperature-conditioned medium activated with (NH₄)₂S0₄ (Jackson et al. (1995); Tarantini et al. (1995)), experiments were designed utilizing the limited SDS-PAGE system in which SDS is removed from the sample buffer to evaluate the co-localization of FGF-1 and Syn-1. The reasoning was that if sufficient SDS was present in the sample buffer to electrophoretically resolve the sample as a function of Mᵣ, it would be insufficient to disrupt any potential pL:protein interaction.

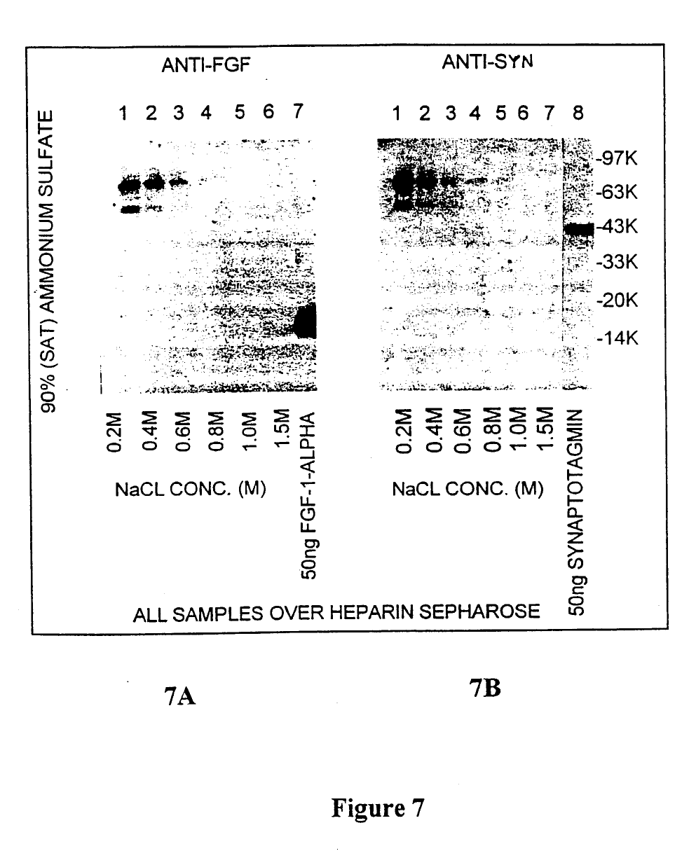

Following the protocol described in Example 2, an FGF-1 immunoblot analysis was conducted using the non-reduced, limited SDS-PAGE system to resolve (NH₄)₂S0₄ activated heat shock conditioned medium from NIH 3T3 cell FGF-1 transfectants, previously absorbed to heparin-Sepharose and gradient eluted with NaCl. As shown in FIG. 7B, FGF-1 was identified as a high Mᵣ complex in the low NaCl elution fractions (<0.6M) and as a low Mᵣ protein in the high NaCl elution fractions (>1.0M).

After this immunoblot was stripped, the same blot was probed with a Syn-1 antibody. As shown in FIG. 7B, the low Mᵣ FGF-1 signal (Lane 6) and the recombinant FGF-1 standard (Lane 7) were not detected; however, the high Mᵣ signals previously detected with an FGF-1 antibody were also detected by the Syn-1 antibody only in those samples which were eluted from immobilized heparin as low ionic strength heparin-binding proteins (Lanes 1,2,3). The combined data indicate that both FGF-1 and Syn-1 can be co-localized using the non-reduced, limited SDS-PAGE system as high Mᵣ complexes eluting from heparin-Sepharose with a NaCl gradient profile similar to that described in FIG. 4 for the FGF-1 homodimer. Indeed, these data support the initial premise that FGF-1 is able to interact with Syn-1 in heat shock conditioned medium. Further, purification of FGF-1 from the 0.2-0.4M NaCl-eluted fractions has been shown to resolve FGF-1 in these fractions as a homodimer (Jackson et al. (1995)).

### Example 6

### Stable Antisense (Γ)-Syn-1, Sense FGF-1 NIH 3T3 Cell Co-Transfectants Exhibit Poor Growth, an FGF-1 Growth Response and Do Not Export FGF-1 in Response to Heat Shock

To further evaluate the role of Syn-1 in the heat shock-induced FGF-1 export pathway, the NIH 3T3 cell FGF-1 transfectants were co-transfected with an antisense (γ) Syn-1 cDNA (1410 bp) designed to repress the translation of the endogenous Syn-1 mRNA using a hygromycin-resistant expression plasmid. The reasoning behind this experiment was that if Syn-1 was involved in the regulation of the FGF-1 export pathway, it should be possible to at least attenuate the release of FGF-1 in response to temperature stress under conditions where the translation of the Syn-1 mRNA is repressed. A Syn-1 cDNA fragment encoding the Syn-1 open-reading frame was used for these studies rather than the more traditional antisense-AUG oligomer approach because the latter method is more appropriate for the translational repression of low abundance transcripts; plus stable clones could be obtained for further biochemical analysis.

The experimental design and protocols were identical to those described in Jackson et al. (1995), and Tarantini et al. (1995). Two stable clones were evaluated including one in which the intracellular levels of FGF-1 were reduced as a result of the selection process. The pXZ38 cells were used as control transfectants, and #10 Syn-1 and #19 Syn-1 are the low and high FGF-1 expressing γ-Syn-1, FGF-1 NIH 3T3 cell co-transfectants, respectively.

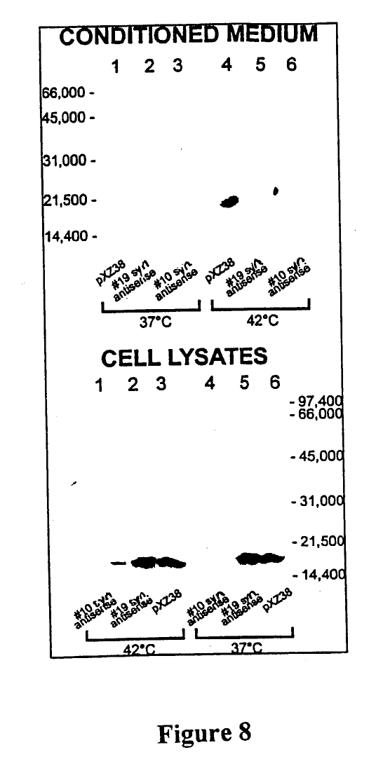

As shown in FIG. 8, FGF-1 was not observed in medium conditioned by heat shock in either of the co-transfectant clones, suggesting that the repression of Syn-1 translation inhibits the release of FGF-1 in response to heat shock. This further supports the conclusion that Syn-1 is involved in the regulation of FGF-1 export.

FIG. 8 shows that antisense (γ) Syn-1 represses the release of FGF-1 in response to heat shock. Immunoblot analysis of conditioned medium from heat shocked FGF-1 NIH 3T3 transfectants cotransfected with antisense (γ) Syn-1 cDNA, as compared with control conditioned media; and cell lysates under comparable conditions. pXZ38 cells=control transfectants; #10 Syn-1 and #19 Syn-1 are low and high FGF-1 expressing γ-Syn-1, FGF-1 NIH 3T3 cell co-transfectants, respectively. The experimental design and procedures used are as described in Jackson, et al., J Biol. Chem. 270:33-36 (1995) and Tarantini, et al., J. Biol. Chem. (1995)).

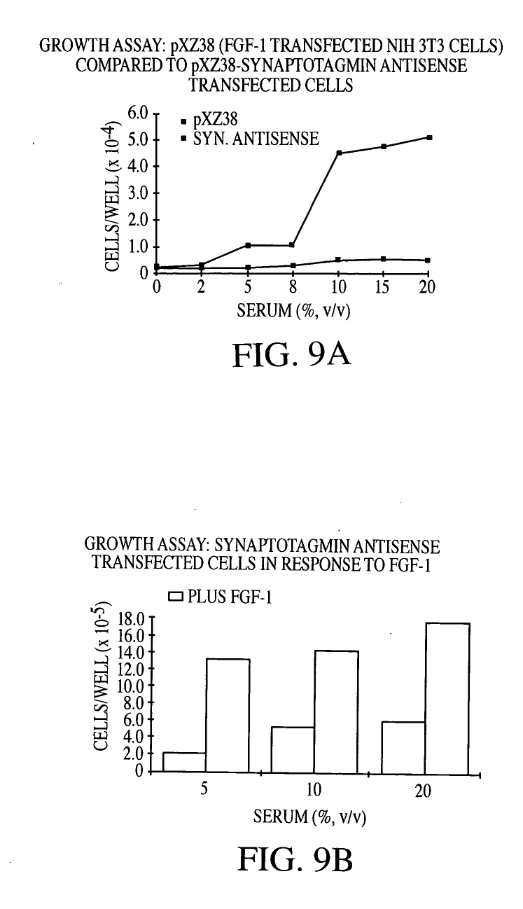

Additionally, the γ-Syn-1, sense FGF-1 co-transfectants grew slowly in response to serum (FIG. 9). A fetal bovine serum (FBS) growth response curve shows FGF-1 and γ-Syn-1, FGF-1 co-transfectants as a function of FBS (FIG. 9A); and a second curve shows the growth response of γ-Syn-1, FGF-1 co-transfectants (FIG. 9B) as a function of FBS in the presence and absence of FGF-1 (10 ng/ml containing 5 μg/ml heparin). The cells were seeded at a density of 1×10⁴ cells/cm², and a viable cell number was reported after 5 days.

FIG. 9 also shows that the addition of exogenous FGF-1 to these cells significantly increased their ability to grow in the presence of 20% FBS. When tested, stable γ-Syn-1 NIH 3T3 cell transfectants (lacking the FGF-1 cDNA) exhibited similar growth properties.

### Example 7

### The FGF-1 Export Pathway Does Not Restrict the Release of a Large Molecular Weight (Mᵣ) Form of FGF-1

In order to determine whether a size restriction exists preventing high Mᵣ complexes from exiting the cell, a chimera was prepared in which the reporter gene, β-galactosidase (gal), was ligated to the carboxy-terminus of FGF-1 (COOH-terminal β-gal chimera) as previously described (Zhan et al., Biochem. Biophys. Res. Commun. 188:982-991 (1992)). In addition, another chimera was also constructed in which the nuclear localization signal (NLS) from the SV40 large T antigen (SV40T) gene was ligated at the amino-terminus of FGF-1:β-gal (NH₂-terminal SV4OT NLS COOH-terminal β-gal chimera). These constructs were transfected into NIH 3T3 cells and stable transfectants obtained and examined for the cytosolic and nuclear presence of the reporter gene using either immunofluorescence microscopy with an anti-β-gal antibody or X-gal staining as described by Zhan et al. (1992).

As shown in FIG. 10, X-gal and immunofluorescence staining for the β-gal reporter gene demonstrates cytosolic staining for the FGF-1:β-gal chimera, but nuclear staining for the SV40T-NLS:FGF-1:β-gal protein. In FIG. 10A, the reporter gene may be seen in the nucleus of the SV4OT NLS:FGF-1:β-gal transfectants and in the cytosol of the FGF-1:β-gal transfectants.

The stable transfectants were also subjected to heat shock (2 hrs, 42° C.), and the conditioned medium processed for β-gal immunoblot analysis as described by Jackson et al. (1995), and Tarantini et al., J. Biol Chem. (1995) (see, Example 6), and immunoblot analysis for β-gal performed. The cells were lysed in boiling SDS which does not conserve the nuclear compartment and the total cell lysate processed as described by Zhan et al. (1992).

As shown in FIG. 10B, while FGF-1:β-gal was readily detected in all NIH 3T3 cell lysates and in the heat shock conditioned medium, the SV40T-NLS:FGF-1:β-gal protein was only present in the NIH 3T3 cell lysates. The fact that the SV40T-NLS:FGF-1:β-gal protein was not detected in the heat shock conditioned medium, indicates that intranuclear FGF-1 is probably not accessible to the components involved in regulating the FGF-1 export pathway. Thus, placement of intracellular FGF-1 in an organelle which restricts its access to Syn-1, completely attenuates the temperature-induced FGF-1 export pathway. However, the presence of FGF-1:β-gal in heat shock conditioned medium as a Mᵣ˜118 kDa chimera argues that the mechanism used to release FGF-1 does not exclude high Mᵣ forms of FGF-1. Thus, high Mᵣ FGF-1:Syn-1-containing complexes are probably restricted from the FGF-1 export pathway.

In FIG. 10B, the FGF-1:β-gal and SV40T NLS:FGF-1 β-gal both appear to migrate with an apparent Mᵣ 118 kDa since the SV40T NLS only contributes 6 amino acid residues to the total mass of the chimera.

### Example 8

### The Identification of Putative FGF-1-Associated Proteins Present in the Temperature-Conditioned Medium of FGF-1:B-Gal NIH 3T3 Cell Transfectants

Because the presence of the FGF-1:β-gal chimera could not be resolved in medium conditioned by heat shock (FIG. 10B) as a structurally intact protein, experiments were designed to determine whether immunoprecipitation strategies could be utilized to resolve the presence of proteins associated with FGF-1 using (³⁵S)-met/cys labeled transfectants. There was concern about using either Syn-1 or FGF-1 antibodies for this purpose because similar strategies employed in receptor-mediated signaling, DNA and mRNA transacting factors, and intracellular trafficking arts have suggested that antibodies against the protein of interest that are used to immunoprecipitate the protein of interest as a non-covalent multi-protein complex can disrupt the conformational integrity of the complex, and thus irreversibly modify associative protein:protein interactions. Consequently, the FGF-1:β-gal chimera secretion data produced by these experiments are particularly significant because they indicate the possibility of using β-gal antibodies for this purpose in a non-disruptive manner since β-gal-transfected NIH 3T3 cells do not release β-gal into the conditioned medium in response to heat shock (data not shown).

Moreover, immunoprecipitation of (³⁵S)-met/cys-labeled γ-gal NIH 3T3 cell transfectants resolved only the presence of β-gal as a radiolabeled protein from cell lysates prepared by Dounce homogenization (data not shown). Thus, it was determined that if β-gal immunoprecipitation from either cell lysates or medium conditioned by heat shock of FGF-1:β-gal NIH 3T3 cell transfectants were able to resolve multiple radiolabeled proteins, the bands would presumably represent proteins associated with FGF-1, and not β-gal. Of course, one cannot eliminate the possibility that the protein resolved by β-gal immunoprecipitation are associated with both FGF-1 and β-gal.

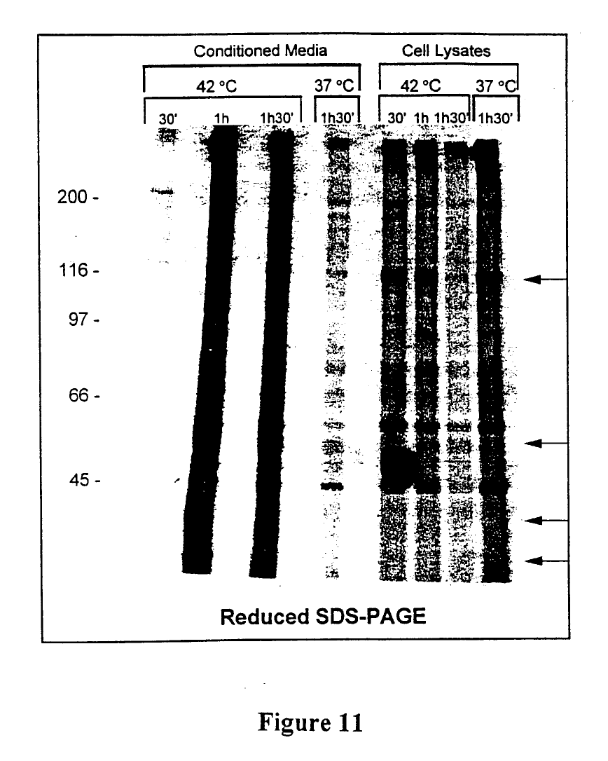

Thus, FGF-1:β-gal NIH 3T3 cell transfectants were metabolically labeled with (³⁵S)-met/cys and subjected to heat shock (42° C., 2 hr). Cell lysate and conditioned medium were immunoprecipitated with anti-β-gal antiserum and analyzed by SDS-PAGE. As shown in FIG. 11, the immunoprecipitate resolved not only the presence of the FGF-1:β-gal chimera (Mᵣ˜118 kDa) in the medium conditioned by heat shock, and not in control conditioned medium; it also resolved additional polypeptides with apparent Mᵣs of approximately 70, 40 and 20 kDa (marked with arrows in FIG. 11). Similar radiolabeled bands were identified in the heat shock cell lysate but not in the control lysate. The combined data suggest that these bands probably represent proteins associated with each other as a high Mᵣ complex; and since the FGF-1:β-gal chimera is the target of this complex, they are presumed to be FGF-1:β-gal-associated proteins.

### Example 9

### The Identification of an S100 Family Member and Other Proteins Associated with the FGF-1:Syn-1 Complex

In view of the determination, as described herein, that a p40 fragment of Syn-1 is complexed with the FGF-1 homodimer in medium from heat shocked FGF-1 NIH 3T3 cells transfected in vitro, and further in view of the occurrence of FGF-1 derived from bovine tissue extracts as a high molecular weight, non-covalent complex (Burgess et al., 1985 J. Biol. Chem. 260:11389), the FGF-1:Syn 1 complex was further examined to determine whether or not other proteins were associated with the complex.

Ten (1.5 kg) unstripped ovine brains (Pel-Freeze® Biologicals, Rogers, Ariz.) were homogenized in 1.3 volumes of 50 mM Tris-CHI, pH 7.4, for 2 min in a Waring blender. The homogenate was centrifuged at 10,000×g for 1 h, and the supernatant was filtered through sterile gauze. The filtrate was subjected to stepwise salt fractionation with 50 and 95% (HN₄)₂SO₄ saturation, and the precipitates were collected by centrifugation as described (Maciag et al., 1979 Proc. Nat. Acad. Sci. U.S.A. 76:56-74). The 95% (NH₄)₂SO₄ saturation precipitate was resuspended in 100 ml of 50 mM Tris-HCl, pH 7.4, and dialyzed for 18 h against 50 volumes of the resuspension buffer using a Spectra/Por (M₂ 12-14,000) dialysis membrane (Spectrum Medical Industries Inc., Houston, Tex.). All purification procedures were performed at 4° C.

A 2.4×22-cm plastic column containing 25 ml of hydrated heparin-Sepharose™ CL-6B was equilibrated with 10 volumes of 50 mM Tris-HCl, pH 7.4, and the brain extract was absorbed twice over the immobilized heparin. The column was washed with at least 10 bed volumes of the resuspension buffer until the absorbance of the eluate at λ=280 nm was less than 0.01. Three batch fractions were eluted with 100 ml of 50 mM Tris-HCl, pH 7.4, containing 0.4 M NaCl, 07 M NaCl, and 1.5 M NaCl, and samples from each NaCl eluate (25 ml) were absorbed to a C4 column (Vydac™, Hesperia, Calif.) conditioned in 0.1% trifluoroacetic acid (Pierce, Rockford, Ill.). RP-HPLC was performed as described (Burgess et al, 1985. Biol Chem. 260:11389) using a linear gradient of acetonitrile (40 to 100%) in 0.1% (v/v) trifluoroacetic acid at a flow rate of 1 ml/min, and the effluent was monitored at λ=214 nm. Samples were collected as absorbance peaks independent of volume in 1.0 M Tris-HCl, pH 7.4, in an attempt to maintain aggregate integrity and analyzed by FGF-1 and Syn-1 immunoblot analysis as described herein and in Jackson et al (1992 Proc. Nat. Acad. Sci. U.S.A. 89:10691) except that the ECL (Amersham, Arlington Heights, Ill.) system was used for protein detection. Although many peaks exhibited the presence of both FGF-1 and Syn-1 by immunoblot analysis, only the 1.5 M NaCl heparin-Sepharose elution fraction contained a unique absorbance peak that contained both FGF-1 and Syn-1 by immunoblot analysis at a dilution of 1:100. This peak was re-chromatographed on a microbore 300-A C4 Aquapore™ RP300 column (Perkin-Elmer, Norwalk, Conn.), and bound proteins were eluted as absorbance peaks at λ=214 nm with a linear gradient (40-100%) of 70% (v/v) acetonitrile in 0.1% (v/v) trifluoroacetic acid and a flow rate of 0.2 ml/min.

Approximately 10 μg of protein from each major peak were subjected to proteolytic digestion using lysyl endopeptidase C (Boehringer Mannheim, Indianapolis, Ind.) as described (Egerton et al, 1992. Immunol 149:1847). Peptides were isolated-by RP-HPLC using an Applied Biosystems (Foster City, Calif.) model 130 separation system as instructed by the manufacturer. Isolated peptides were subjected to automated Edman degradation using either an Applied Biosystems model 473A or 477A protein sequenator, according to the manufacturer's recommendations. Proteins were identified by comparison of the amino acid sequences obtained for several of those peptides against an NCBI (National Center for Biotechnology Information) protein sequence data base using the BLAST (Basic Local Alignment Search Tool) program.

Immunoblot analysis of fractions resolved by heparin affinity chromatography identified a fraction of ovine brain extract in which both FGF-1 and Syn-1 were present. This fraction, when further resolved by RP-HPLC, revealed multiple fractions containing FGF-1, Syn-1, as well as a fraction containing both FGF-1 and Syn-1. See, FIG. 14, top panel.

Since the fraction containing both FGF-1 and Syn-1 potentially represented a complex of these two proteins, an assay was undertaken to separate the FGF-1 :Syn-1 fraction from the other containment proteins, if any. Thus the fraction containing both FGF-1 and Syn1 was collected, and further resolved by RP-HPLC. As shown in the middle panel of FIG. 14, the fraction containing both FGF-1 and Syn-1 eluted with a retention time similar to that seen in the previous observation (FIG. 14, top panel), thus confirming the presence of the FGF-1 and Syn-1 proteins in this fraction.

Since the fraction represented a potentially stable complex between FGF-1 and Syn-1, a study was designed to determine whether FGF-1 and Syn-1 could be dissociated using a denaturant. Consequently, the fraction was treated with 8M guanidinium HCl, a chaotropic agent, and the fraction was again resolved by RP-HPLC. As shown in the bottom panel of FIG. 14, multiple peaks were generated from the putative FGF-1:Syn-1 complex, including peaks with retention times similar to that which had previously been identified as FGF-1 and Syn-1. In addition, the FGF-1 and Syn-1 analysis confirmed the identity of these peaks.

Surprisingly, however, additional peaks with different elution retention times were also generated by treatment of the FGF-1:Syn-1 complex with a chaotropic agent. This demonstrated that FGF-1 is not only complexed with Syn-1 in neutral pH extracts of ovine brain, but also with other proteins. Further, it was determined that this complex is remarkably stable to exposure to RP-HPLC solvents.

Conventional protein sequencing methods, however, failed to identify the other proteins associated with either FGF-1, Syn-1 or both in the Syn-1:FGF-1 complex (FIG. 14, bottom panel). Moreover, Edman degradation of the individual peaks resolved by RP-HPLC did not yield an NH₂-terminal sequence, showing that each of the components of the FGF-1:Syn-1 complex contain a blocked NH₂-terminus, including FGF-1 and Syn-1. This determination suggested that the purification of the multiprotein FGF-1:Syn-1 complex can be achieved under conditions which do not result in the hydrolytic modification of the NH₂-terminus of any component of this complex.

Since the identity of the individual protein components of the FGF-1:Syn-1 complex could not be accessed by direct NH₂-terminal sequence analysis, peptide maps of those peaks exhibiting prominent absorbance profiles were obtained. Analysis of the most prominent peaks by NH₂-terminal sequence analysis of multiple proteinase K-derived peptides identified the first peak to be identical to the ovine homolog of human S100A13. Similar analysis of the second prominent absorbance peak demonstrated it to be identical to the ovine homolog of human cyclophilin B. Thus, the present data clearly identifies S100A13 and cyclophilin B to be components of the FGF-1:Syn-1 complex.

S100A13 is a recently identified member of the human S100A gene family with well conserved structural features. (Wicki et al., 1996 Biochem. Biophys. Res. Commun. 227:594; Schafer et al., 1996 Trends Biochem. Sci. 21:134) Characterization of the isolated S100A13 gene has shown that it contains the prerequisite structural motifs identifying the S100A gene family members as Ca²⁺, calmodulin-, and acidic phospholipid-binding proteins. In addition, S100A13 is a cysteine-free structure which is exported even though, like FGF-1, it lacks a classical signal peptide sequence to direct it through the conventional ER-Golgi apparatus-mediated exocytotic pathway. Nevertheless, S100A proteins are well characterized as a calmodulin-binding protein, and are involved in the regulation of the filamentous cytoskeleton. Data suggest that FGF-1 may utilize the cytosolic side of conventional Syn-1 containing vesicles for traffick to the inner surface of the plasma membrane. Thus, S100A13 may serve as a conduit between the exocytotic organelle and the calmodulin-rich F-actin cytoskeleton. In addition, data suggest that (i) the biologic activities previously associated with the S100A gene family members may actually be the result of an FGF gene family contaminant found in relatively low specific activity preparations, and (ii) SI OOA antagonists could prove useful to limit the release of FGF-1.

The other component of the multiprotein complex, cyclophilin B (CpB) is a cyclosporin A1 (CSA)-binding protein, which was initially identified as a heat shock-induced peptidylproline CIS-trans isomerase (hsp40) involved in protein-folding and protein-protein interactions. CsA is well recognized as an immune suppressor in humans, having novel tissue/organ-specific side effects, including enhanced peripheral nerve repair in man.

Thus, the data provide evidence to support an association of Syn-1 and FGF-1 as a multiprotein complex in neural tissue in vivo, and to confirm the characterization of the FGF-1:Syn-1 complex derived from in vitro systems. In addition, because two additional proteins present within the FGF-1:Syn-1 complex have been identified as S100A13 and cyclophilin B, it is assumed that agents which agonize or antagonize this association will provide useful reagents for the inhibition and/or activation of the FGF-1 secretion pathway in vitro and in vivo.

### Example 10

### Isolating Syn-1-Cleaving Enzyme (Syn-1 Ce, Syn-1 Protease)

Because the data in FIG. 7 indicated that Syn-1 and FGF-1 are present in heat-shocked condition medium as a high Mᵣ complex which elutes from heparin-Sepharose at 0.4M NaCl, this system was tested. A neutral extract of bovine brain was prepared as described by Jaye et al. (1986), and resolved by 0.4M NaCl elution from heparin-Sepharose. The 0.4M NaCl eluted fractions were pooled, lyophilized, and the fraction resolved by Sephadex G-100 gel exclusion chromatography at pH 7.0 as previously described by Maciag et al. (1982). The fractions were pooled and subjected to either immunoprecipitation with a Syn-1 antibody followed by immunoblot analysis using an FGF-1 antibody (data not shown) or immunoprecipitation with an FGF-1 antibody followed by reduced immunoblot analysis using a Syn-1 antibody (FIG. 13).

The samples from the Sephadex G-100 column were Western blotted as described by Zhan et al., J. Biol Chem. 269:20221-20224 (1994). The gel on the left of FIG. 13 represents FGF-1 immunoprecipitation followed by Syn-1 immunoblot analysis. The gel on the right of FIG. 13 represents Syn-1 immunoprecipitation followed by Syn-1 immunoblot analysis.

If the high Mᵣ fractions contained FGF-1 homodimer:Syn-1 complex, it is possible to not only visualize FGF-1 in the Syn-1 immunoprecipitate, but also to visualize a Syn-1 fragment in the FGF-1 immunoprecipitate. Indeed, it was possible to detect FGF-1 in the Syn-1 immunoprecipitate and the p40 Syn-1 fragment in the FGF-1 immunoprecipitate in the high Mᵣ Sepharose G-100 fractions (FIG. 13). The combined data not only confirm the data derived from the cell culture effort relative to the association between a Syn-1 fragment and FGF-1 using a physiologic relevant organ, but they also predict that the neutral pH brain extract may contain a protease or proteolytic-type of a complex, which was able to successfully modify p65 Syn-1 and convert it to p40 Syn-1.

The Syn-1-CE open-reading frame is defined and characterized as previously described for other novel cDNAs (see, e.g., Zhan et al. (1993); Hla et al., Biochem. Biophys. Res. Comm. 167:637-643 (1990); Hla et al., J. Biol. Chem. 265:9308-9313 (1990)), including the identification of putative structure-function domains, motifs and/or sites. This provides an appropriate resource for the expression of Syn-1-CE as a recombinant protein using the pET3c prokaryotic expression plasmid either as a non-modified protein or as a GST fusion protein as described (Zhan et al. (1994)).

The Syn-1-CE recombinant is purified using either the GST tag or by conventional protease affinity methods, permitting this resource to be used not only for the generation of Syn-1-CE antibodies, but also as an (¹²⁵I)-ligand to probe to determine whether Syn-1 and/or FGF-1 homodimer/monomer associate with Syn-1-CE using reduced/nonreduced immunoprecipitation/immunoblot analysis as described in FIG. 13. The Syn-1-CE antibody will also prove useful to study the biosynthesis of Syn-1-CE using conventional pulse-chase in either ECV and/or SMC FGF-1₁₋₁₅₄ transfectants relative to the synthesis of Syn-1.

### Example 11

### Temperature Stress-Induced FGF-1/Syn-1 Complex Export Depends on Extravesicular Syn-1 Domains

In this Example, NIH-3T3 cells transfected with a recombinant gene construct encoding a fusion protein comprising FGF-1 joined through its carboxy terminus to the reporter molecule β-galactosidase (β-gal) (Zhan et al., Biochem. Biophys. Pes. Commun. 188:982, 1992) were cotransfected with recombinant DNA constructs encoding Syn-1 or Syn-1 deletion mutants to assess the contribution of Syn-1 domains structurally defined by their positions within the Syn-1 encoding sequence to the export of FGF-1:Syn-1 complexes by cotransfectants in response to temperature stress.

Plasmid isolation, production of competent cells, transformation and bacteriophage manipulations were carried out according to published procedures (Sambrook et al., Molecular Cloning, a Laboratory Manual, Cold Spring Harbor Laboratory Press, Cold Spring Harbor, N.Y., 1989). Stable transfection of NIH-3T3 cells with the FGF-1-β-gal construct, and export into the conditioned medium of the FGF-1-β-gal fusion protein by these cells in response to heat shock, were as described above in Example 7. Briefly, NIH 3T3 cells obtained from the American Type Culture Collection (ATCC, Bethesda, Md.) were stably transfected with a plasmid containing the FGF-1:β-gal construct as described (Shi et al., J. Biol. Chem. 272:1142, 1997). A plasmid encoding the full length p65 form of rat Syn-1 comprising amino acids 1-421 (SWISS-PROT Accession Number P21707; Perin et al., 1990 Nature 345:260) was provided by Dr. T. C. Sudhof (Univ. of Texas Southwestern Medical Center, Dallas, Tex.) and cloned into the PCRII vector (InVitrogen Corp., San Diego, Calif.) after generating a Nde I site at the amino terminus encoding sequence, and a Bam HI site at the carboxy terminus encoding sequence of the p65 insert by PCR, using primers complementary to the ends of the p65 encoding sequences and to the desired restriction enzyme recognition sites according to well known procedures.

A Syn-1 truncation deletion mutant, p65(Δ120-214), comprising the p65 coding sequence from which the nucleotides encoding amino acids 120-214 have been deleted, was made by removing the Afl II-Kpn I fragment from the p65 Syn-1 construct resulting in the loss of amino acids 120-214 and the addition of the tripeptide sequence GID between amino acids 119 and 215. A second Syn-1 truncation deletion mutant, p40, comprising the p65 coding sequence from which the nucleotides encoding amino acids 1-118 have been deleted, was made by removing the 5′ sequences at the AfI II site of the p65 nucleotide sequence. The p40 product thus comprises Syn-1 amino acids 119-421 plus the Myc epitope tag at the carboxy terminus. For transfection analysis, p65 Syn-1, p65(Δ120-214) and p40 were cloned into the pMEXhygro vector, which was generated from the pMEXneo vector (Martin et al., Lab. Invest. 23:86, 1970) by removing the Stu I-Nru I fragment containing the neomcin resistance gene and inserting a 1774 base pair Hpa I-Eco 31 fragment containing the hygromycin B resistance promoter and gene from the p3'SS vector (Stratagene, San Diego, Calif.).

Stable NIH 3T3 FGF-1:β-gal transfectants were co-transfected with either p65 Syn-1, p65(Δ120-214) or p40 and maintained in Dulbecco's modified Eagle's medium (DMEM, GIBCO-BRL, Grand Island, N.Y.) containing 10% (v/v) bovine calf serum (BCS, Hyclone, Logan, Utah), 1× antibiotic-antimycotic (GIBCO-BRL) and 200 μg/ml G418 (GIBCO-BRL) and 250 μg/ml hygromycin (Boehringer Mannheim, Indianapolis, Ind.) on sterile tissue culture dishes coated with 10 μg/cm² human fibronectin prepared from human plasma by affinity adsorption to a column of immobilized type I collagen by methods well known in the art. Confluent monolayers of co-transfectants were rinsed with DMEM containing 4 U/ml heparin (Upjohn, Kalamazoo, Mich.) and subjected to heat shock (42° C. for 100 min) in DMEM containing 4 U/ml heparin and 0.5% BCS as described (Jackson et al., Proc. Nat. Acad. Sci. USA 89:10691, 1992). For each Syn-1 construct, three independent co-transfectant clones were tested.

Following heat shock, conditioned media were collected, filtered using 0.2 μm filters (Millipore, Bedfore, Mass. ) according to the supplier's recommendations, and activated with 0.1% (w/v) dithiothreitol (DTT, Sigma, St. Louis, Mo.) for 2 h at 37° C. (Jackson et al., J. Biol. Chem. 270:33, 1995). Conditioned media were then passed over a column of heparin-Sepharose™ CL-6B (Pharmacia Biotech, Piscataway, N.J.) equilibrated and eluted with 50 mM Tris pH 7.4 containing 10 mM EDTA (TE buffer, TEB). Following TEB washing, the column was eluted with 1.5 M NaCl in TEB and the eluate collected and concentrated using a Centricon™ 10 concentrator (Amicon, Inc., Beverly, Mass.) according to the manufacturer's instructions. Aliquots of the concentrated eluates were analyzed by 10% sodium dodecylsulfate polyacrylamide gel electrophoresis (SDS-PAGE) as described (Jackson et al., J. Biol Chem. 270:33, 1995). Electrophoretically separated samples were further characterized by immunoblot analysis as described (Jackson et al., Proc. Nat. Acad. Sci. USA 89:10691, 1992). Briefly, following electrophoretic transfer of resolved components to nitrocellulose filters, the filters were incubated for 2 h at 42° C. in TCB (24 mM Tris pH 7.4, 136 mM NaCl, 2 mM KCl, 0.1% (v/v) Tween 20) (Sigma, St. Louis, Mo.) containing 5% (w/v) nonfat dry milk. Filters were probed for 1 h at room temperature with antibodies diluted in TCB containing 5% (w/v) nonfat dry milk, washed with TCB and developed using the ECL chemiluminescent detection system (Amersham, Cleveland, Ohio) according to the manufacturer's instructions.

Antibodies were either 5 μg/ml polyclonal rabbit anti-human FGF-1 antibodies (Sano et al., J. Cell Biol. 110:1417, 1990) or 5 μg/ml polyclonal rabbit anti-rat Syn-1 antibodies. Immunogens for the production of anti-Syn-1 antibodies were recombinant rat Syn-1 polypeptides liberated by thrombin cleavage from glutathione S-transferase fusion proteins comprising rat Syn-1 amino acids 96-421 fused to GST as described (Bennett et al., J. Neurosci. 13:1701, 1993) except that strain BL21 E. coli used to express the fusion proteins were lysed with 10 μg/ml lysozvme in 50 mM Tris pH 8.8, 10 mM EDTA, 10 mM glucose. Thrombin-cleaved proteins were further purified by absorption to heparin-Sepharose™ equilibrated in 50 mM phosphate pH 7.5 and elution using a gradient of 0-1.5 M NaCl in the same phosphate buffer. Rabbit anti-rat Syn-1 antibodies were generated by immunizing six-month old female New Zealand white rabbits (Hazelton Research, Madison, Wis.) with recombinant rat Syn-1 polypeptides, 1 mg of protein emulsified in complete Freund's adjuvant (Calbiochem, San Diego, Calif.) injected intradermally and followed by 200 μg booster immunizations in incomplete Freund's adjuvant (Calbiochem) according to standard protocols. Immune sera were collected and affinity purified by binding to purified recombinant rat Syn1(96-421)immobilized on a Problot PVDF membrane (Applied Biosystems, Inc., Foster City, Calif.) that had been pre-blocked at 42° C. with 5% (w/v) bovine serum albumin in TCB. The membrane was washed 3 times with 0.05% (v/v) Triton-X100 in 50 mM Tris pH 7.4, 150 mM NaCl, the antibodies eluted from the membrane using 0.2M glycine pH 2.8, and the recovered antibody solution was then neutralized with 1 M K₂HPO₄ pH 7.4.

The p40 Syn-1 proteolytic cleavage product appeared in heat shock conditioned media from NIH-3T3 FGF-1-β-gal:p65 Syn-1 co-transfectants with the same kinetics as FGF-1-βgal, a result that was unchanged when culture media were supplemented with protease inhibitors during the heat shock treatment. Conditioned media were also assayed for lactate dehydrogenase (LDH) activity as a measure of cell lysis during the heat shock treatment (Bergmeyer, in Methods of Enzymatic Analysis, H.-U. Bergmeyer, ed., 1965 Academic Press, NY, pp. 736-743), and the percentage of total cell lysate LDH activity in conditioned media from heat shocked cells (8.5%) was not significantly higher that that in cells maintained at 37° C. for a comparable duration (5.6%).

To rule out possible dependence of p40 Syn-1 export from cells on cytosolic FGF-1 expression, levels of p40 in heat shock conditioned media from two p65 Syn-1 transfected 3T3 cell lines that do not express high levels of FGF-1 were examined. In p65 Syn-1 NIH 3T3 transfectants, which express only low levels of endogenous FGF-1, and in p65 Syn1 NIH 3T3 cells co-transfected with a Cys-free mutant form of FGF that is not exported (Jackson et al., J. Biol Chem. 270:33, 1995), p40 release following heat shock was similar to that observed in the NIH 3T3 FGF-1-β-gal:p65 Syn-1 co-transfectants.

Examination of the effects of specific pharmacologic inhibitors on the heat shock induced release of p40 demonstrated that, as has been previously reported for FGF-1 release (Jackson et al., Proc. Nat. Acad. Sci. USA 89:10691, 1992), p40 release from NIH 3T3 FGF-1-β-gal:p65 Syn-1 co-transfectants is inhibited by the transcription inhibitor actinomycin D and the translation inhibitor cycloheximide, but is unaffected by brefeldin A, an inhibitor of the classical protein secretion pathway for proteins having signal sequences. Also as previously reported for FGF-1 released by cells after heat shock (Jackson et al., 1992), higher levels of heparin binding p40 were detectable in heat shock conditioned media from NIH 3T3 FGF-1-β-gal:p65 Syn-1 co-transfectants when the conditioned media were activated with either ammonium sulfate or DTT.

Conditioned media from NIH 3T3 FGF-1-β-gal:p65(Δ120-214) co-transfectants subjected to heat shock were examined for the presence of Syn-1 derived polypeptides, to ascertain whether the absence of the deleted 95 amino acids from Syn-1 affects the FGF-1 and p40 export pathways. Immunoblot analysis of heat shock conditioned media for Syn-1 products exported from these co-transfectants revealed neither detectable intact Syn-1 p65(Δ120-214) polypeptides nor proteolytic fragments of this deletion mutant. In addition, no FGF-1 was detectable in these conditioned media, demonstrating that the deleted 95 amino acid domain, which contains a calcium binding site, a pS binding site and a casein kinase II phosphorylation site (See, e.g., Sudhoff et al., Neuron 17:379, 1996; Sutton, Cell 80:929, 1995; Brose et al., J. Biol. Chem. 270:25273; and references cited therein.), is required for both Syn-1 and FGF-1 to access the heat shock induced protein export pathway. Examination of NIH 3T3 FGF-1-β-gal:p65(Δ20-214) co-transfectant cell lysates by immunoblot analysis revealed that intracellular proteolytic fragments of p65(Δ120-214) were detectable, and that the cells were unable to export these fragments.

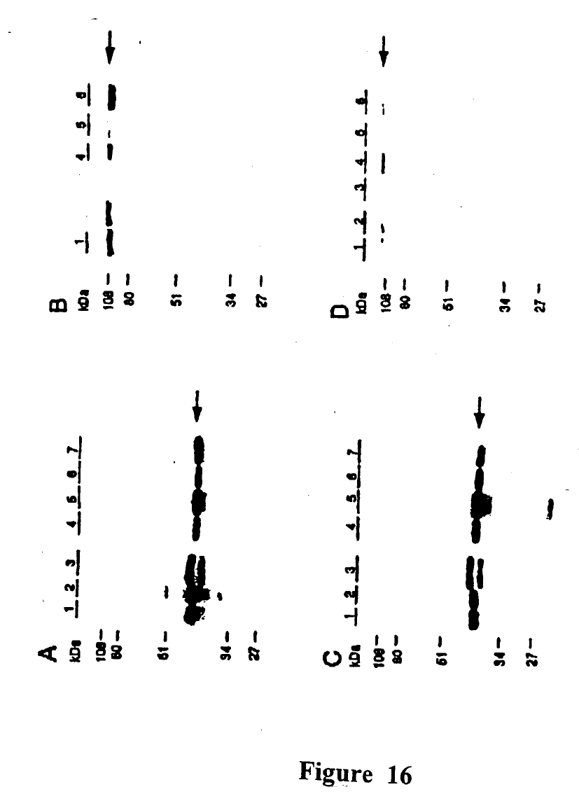

The observation that only the p40 extravesicular portion of the vesicular transmembrane p65 Syn-1 protein was detectable in the medium conditioned by NIH-3T3 FGF-1-β-gal:p65 Syn-1 co-transfectants after heat shock suggested that one or more intracellular proteases cleave p65 Syn-1 as part of the heat shock response. Stable NIH-3T3 FGF-1-β-gal transfectants co-transfected with the Syn-1 p⁴0 construct described above, which lacks Syn-1 amino acids 1-118, were constructed and subjected to heat shock. These NIH-3T3 FGF-1-β-gal:p40 Syn-1 co-transfectants constitutively exported the p40 product even without exposure to heat shock (FIG. 16A, 16B) and heat shocking these co-transfectants potentiated the release of p40 from these cells (FIG. 16C, 16D). The NIH-3T3 FGF-1-β-gal:p40 Syn-1 co-transfectants did not respond to heat shock by releasing increased amounts of FGF-1. Additionally, NIH-3T3 FGF-1-β-gal:p40 Syn-1 co-transfectants did not constitutively release FGF-1.

### Example 12

### S100A13-Binding Amlexanox Interferes with Temperature Stress-Induced Release of FGF-1/Syn-1 Complexes

Characterization of the FGF-1:Syn-1 complex from ovine brain extracts revealed the presence of an ovine homolog of the human S100A13 protein in such complexes, as described above in Example 9. In this Example, the effects of members of the amlexanox family of pharmacologic agents known to bind to S100A13 (Oyama et al., Biochem. Biophys. Res. Commun. 240:341, 1997) were evaluated in cell based assay systems for export of FGF1:Syn-1 complexes in vitro. NIH 3T3 FGF-1-β-gal:p65 Syn-1 co-transfectants were constructed and maintained as described above in Example 11 except that heat shock was at 41.5° C. for 90 min. Cells were heat shocked in the absence or presence of amlexanox (also known as AA673, Amoxanox, and Solfa) or one of three amlexanox derivatives (AA617, AA648 or AA777), all of which were obtained from Dr. G. Goto (Takeda Chemical Industries, Osaka, Japan). Following heat shock, conditioned medium was recovered from cell cultures, activated with DTT, adsorbed to heparin-Sepharose and characterized for the presence of FGF-1-β-gal and Syn-1 in the heparin binding fraction by immunoblot analysis as described in Example 11 and in Jackson et al. (Proc. Nat. Acad. Sci. USA 89:10691, 1992).

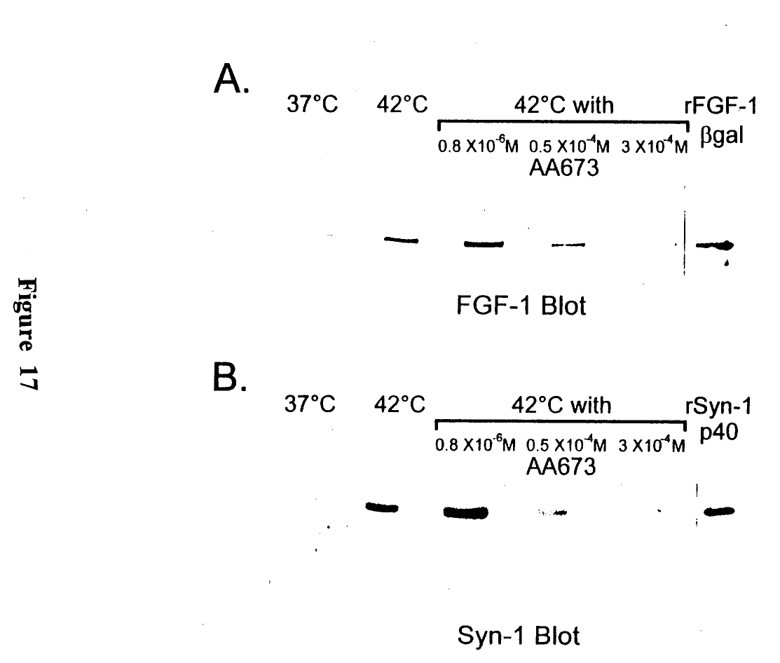

In a concentration dependent fashion, amlexanox inhibited the release of both FGF-1-β-gal and Syn-1 from NIH 3T3 FGF-1-β-gal:p65 Syn-1 co-transfectants in response to heat shock (FIG. 17). Amlexanox concentrations used were within the range where this agent exhibits pharmacologic effects as an anti-allergic and anti-inflammatory drug (See, e.g., Saijo et al, Int. Arch. Allerg. Appl. Immunol. 77:315, 1985; Saijo et al., Int. Arch. Allerg. Appl. Immunol. 78:43, 1985 ; Saijo et al., Int. Arch. Allerg. Appl. Immunol. 79:231, 1986.). Using a different NIH 3T3 cell line stably transfected with FGF-1 instead of FGF-1-β-gal, amlexanox also inhibited FGF-1 release in response to heat shock (not shown). A 3T3 cell line transfected with a chimeric FGF-1 construct having a functional signal sequence for secretion via the classical ER-Golgi protein secretory pathway (Forough et al., J. Biol. Chem. 268:2960, 1993) was unaffected by amlexanox by several phenotypic criteria, including secretory activity (not shown).

Amlexanox was also tested for an effect on the heat shock response of NIH-3T3 FGF-1-β-gal:p40 Syn-1 co-transfectants described above in Example 11. Media were conditioned by two 150 mm dishes of NIH3T3 cells stably co-transfected with FGF-1:β-Gal and rat p40 Syn-1 after exposure to temperature stress (41.5° C., 90 min) with or without treatment. The media were collected and resolved by 10% acrylamide SDS-PAGE under reducing conditions followed by immunoblot analysis for Syn-1 as described herein and in the cited references.

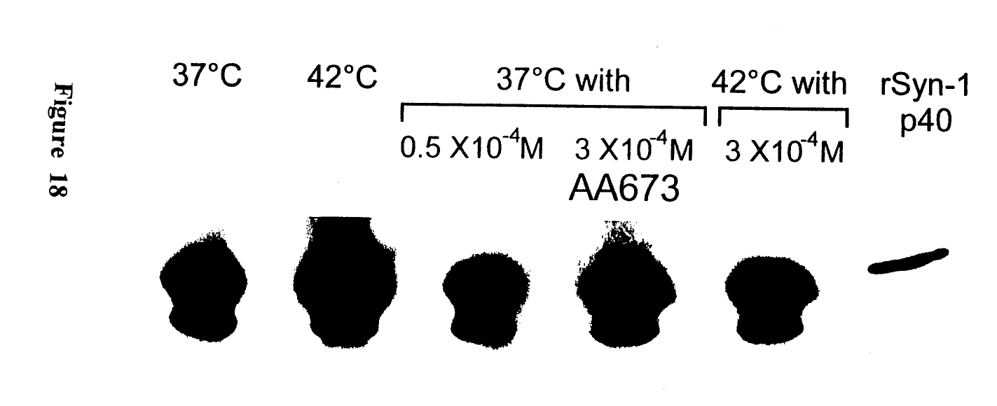

As shown in FIG. 18, amlexanox had no effect on the constitutive release of p40 Syn-1 from these cells, but did inhibit the enhanced p40 export by these cells in response to heat shock. (FIG. 18: Lane 1: media derived from NIH3T3 cell FGF-1:β-Gal and p40 Syn1 co-transfectants maintained at 37° C. for 90 min. Lane 2: media derived from NIH3T3 cell FGF-1:β-gal and p40 Syn-1 co-transfectants following heat shock. Lanes 3 and 4: media derived from NIH3T3 cell FGF-1:β-gal and p40 Syn-1 co-transfectants maintained at 37° C. in the presence of 5×10⁻⁵M, and 3×10⁻¹M AA673 (amlexanox) respectively. Lane 5: media derived from NIH3T3 cell FGF-1:β-gal and p40 Syn-1 co-transfectants following heat shock in the presence of 3×10⁻⁴M, AA673. Lane 6: 50 ng of recombinant rat p40 Syn-1.)

Chemical derivatives of amlexanox were tested for their effects on FGF-1 and p40 Syn-1 release by NIH 3T3 FGF-1 transfectants in response to heat shock. Media were conditioned by three 150 mm dishes of NIH3T3 cells stably transfected with FGF-1α (residues 21-154) after exposure to 41.5° C. for 90 min with or without amlexanox or the indicated amlexanox derivative. The media were collected and resolved by 12.5% acrylamide SDS-PAGE under reducing conditions followed by immunoblot analysis for FGF-1 as described herein.

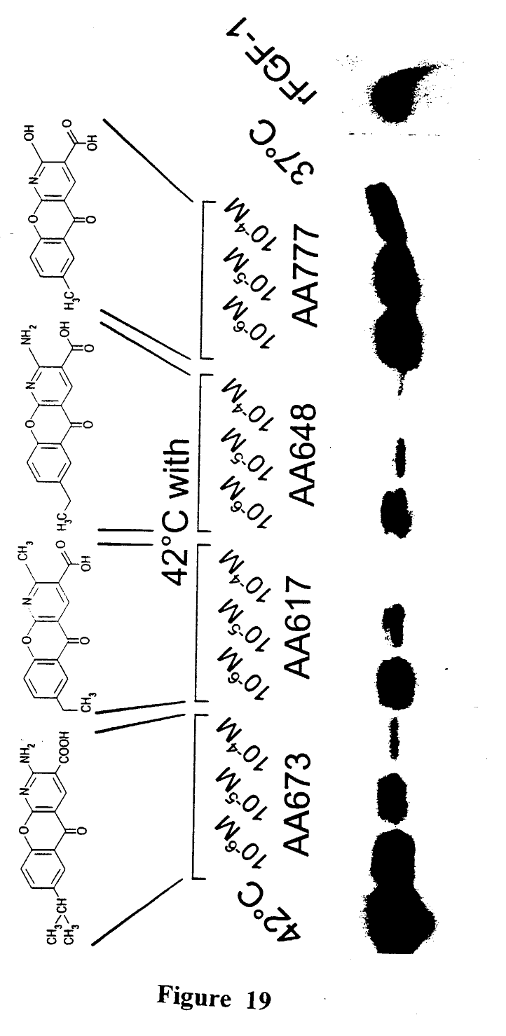

As shown in FIG. 19, immunoblot analysis of media conditioned by cells undergoing heat shock in the presence of amlexanox (AA673) or either of its derivatives AA617 and AA648 revealed inhibition of both FGF-1 and p40 Syn-1 release in response to temperature stress. A third derivative, AA777, did not exhibit these inhibitory effects in these analyses. (FIG. 19: Lane 1: media derived from NIH3T3 cell FGF-1 transfectants following heat shock in the absence of any drug; Lanes 2 through 4: media derived from NIH3T3 cell FGF-1 transfectants following heat shock in the presence of 1×10⁻⁶ M, 1×10⁻⁵ M, 1×10⁻⁴ M, A673 (amlexanox) respectively; Lanes 5 through 7: media derived from NIH3T3 cell FGF-1 transfectants following heat shock in the presence of 1×10⁻⁶ M, 1×10⁻⁵ M, 1×10⁻⁴ M AA617 respectively; Lanes 8 through 10: media derived from NIH3T3 cell FGF-1 transfectants following heat shock in the presence of 1×10⁻⁶ M, 1×10⁻⁵ M, 1×10⁻⁴ M AA648 respectively; Lanes 11 through 13: media derived from NIH3T3 cell FGF-1 transfectants following heat shock in the presence of 1×10⁻⁶ M, 1×10⁻⁵ M, 1×10⁻⁴ M, AA777 respectively; Lane 14: media derived from NIH3T3 cell FGF-1 transfectants maintained at 37° C. for 90 min; Lane 15, 50 ng of recombinant human FGF-1α. The structures of amlexanox (AA673) and its derivatives are also shown.

Although the present invention has been described with reference to the presently preferred embodiments, the skilled artisan will appreciate that various modifications, substitutions, omissions and changes may be made without departing from the spirit of the invention. Accordingly it is intended that the scope of the present invention be limited only by the scope of the following claims, including equivalents thereof.

We claim:

1. A method of regulating FGF-1 export from a cell comprising administering a therapeutic agent in an amount sufficient to inhibit the export of FGF-1, wherein said therapeutic agent inhibits the formation of a complex comprising FGF-1, and wherein the formation of said complex is a prerequisite to FGF-1 export.

2. A method according to claim 1 wherein said complex further comprises Syn-1.

3. A method according to claim 1 wherein said therapeutic agent inhibits the release of FGF-1 from a complex comprising FGF-1 and Syn-1.

4. A method according to claim 1 wherein said therapeutic agent inhibits the release of FGF-1 from, or the association of FGF-1 with, an intracellular vesicle.

5. A method according to claim 1 wherein said therapeutic agent is selected from the group consisting of:
a. an antibody;
b. an immunologically active fragment of an antibody; and
c. an anti-inflammatory agent.

6. A method according to claim 1 wherein said therapeutic agent is selected from the group consisting of a Syn-1 antisense molecule and a Syn-1 antisense molecule encoding agent.

7. A method according to claim 1 wherein said therapeutic agent is selected from the group consisting of a ribozyme that specifically cleaves a Syn-1 encoding nucleic acid and a ribozyme encoding agent, wherein said ribozyme specifically cleaves a Syn-1 encoding nucleic acid.

8. A method of regulating FGF-1 export from a cell comprising administering a therapeutic agent in an amount sufficient to promote the export of FGF-1, wherein said therapeutic agent promotes the formation of a complex comprising FGF-1, and wherein the formation of said complex is a prerequisite for FGF-1 export.

9. A method according to claim 8 wherein said complex further comprises Syn-1.

10. A method according to claim 8 wherein said therapeutic agent promotes the release of FGF-1 from Syn-1 at or near the plasma membrane.

11. A method according to claim 8 wherein said therapeutic agent promotes the release of a complex comprising FGF-1 from an intracellular vesicle.

12. A method according to claim 8 wherein said therapeutic agent promotes the association of a complex comprising FGF-1 with an intracellular vesicle.

13. A method of regulating FGF-1 export, comprising inhibiting a protease that cleaves Syn-1 in an FGF-1:Syn-1 complex, thereby inhibiting the export of said FGF-1:Syn-1 complex.

14. A method of regulating FGF-1 export, comprising increasing the activity of a protease that cleaves Syn-1 in an FGF-1:Syn-1 complex, thereby promoting the release of FGF-1 at or near the plasma membrane.

15. A therapeutic agent which regulates the export of FGF-1 from a cell, wherein said agent:
a. inhibits export of FGF-1;
b. does not inhibit secretion of a leader sequence-containing protein; and
c. inhibits the binding between the FGF-1 and an intracellular vesicle.

16. A therapeutic agent according to claim 15, wherein said agent inhibits the formation of an FGF-1 :Syn-1 complex.

17. A therapeutic agent according to claim 15 wherein said agent inhibits the release of FGF-1 from a complex comprising FGF-1.

18. A therapeutic agent according to claim 15 wherein said agent inhibits the association of FGF-1 with an intracellular vesicle.

19. A therapeutic agent according to claim 15 wherein said agent inhibits the release of FGF-1 from an intracellular vesicle.

20. A therapeutic agent according to claim 15 wherein said agent is selected from the group consisting of:
a. an antibody;
b. an immunologically active fragment of an antibody; and
c. an anti-inflammatory agent.

21. A therapeutic agent according to claim 15 wherein said agent is selected from the group consisting of a Syn-1 antisense molecule and a Syn-1 antisense molecule encoding agent.

22. A therapeutic agent according to claim 15 wherein said agent is selected from the group consisting of a ribozyme that specifically cleaves a Syn-1 encoding nucleic acid and a ribozyme encoding agent, wherein said ribozyme specifically cleaves a Syn-1 encoding nucleic acid.

23. A therapeutic agent according to claim 15 wherein said agent is a protein derived from a recombinant molecule.

24. An isolated, purified protease that cleaves Syn-1 in an FGF-1:Syn-1 complex and which thereby regulates the export of FGF-1.

25. An FGF-1 homodimer:Syn-1 complex.

26. A nucleic acid molecule encoding a truncated Syn-1 molecule, comprising a nucleotide sequence of SEQ ID NO:1 having a deletion.

27. The nucleic acid molecule of claim 26 wherein the truncated Syn-1 molecule lacks a site for cleavage by a protease that cleaves Syn-1 in an FGF:Syn-1 complex.

28. The nucleic acid molecule of claim 26 wherein the truncated Syn-1 molecule lacks a membrane binding site.

29. The nucleic acid molecule of claim 28 wherein the membrane binding site binds to the plasma membrane.

30. The nucleic acid molecule of claim 29 wherein the membrane binding site binds to a cytoplasmically accessible component of a plasma-membrane.

31. The nucleic acid molecule of claim 28, wherein the membrane binding site comprises a phosphatidylserine binding site.

32. A truncated Syn-1 molecule, comprising an amino acid sequence of SEQ ID NO: 2 having a deletion.

33. The truncated Syn-1 molecule of claim 32 wherein the truncated Syn-1 molecule lacks a site for cleavage by a protease that cleaves Syn-1 in an FGF:Syn-1 complex.

34. The truncated Syn-1 molecule of claim 32 wherein the truncated Syn-1 molecule lacks a membrane binding site.

35. The truncated Syn-1 molecule of claim 34 wherein the membrane binding site binds to the plasma membrane.

36. The truncated Syn-1 molecule of claim 35 wherein the membrane binding site binds to a cytoplasmically accessible component of a plasma membrane.

37. The truncated Syn-1 molecule of claim 34 wherein the membrane binding site comprises a phosphatidylserine molecule.

38. A nucleic acid molecule encoding a truncated Syn-1 molecule, comprising a nucleotide sequence of SEQ ID NO:1 having a deletion such that the truncated Syn-1 molecule lacks at least one of the domains selected from the group consisting of a lipid binding domain, a domain containing a site for cleavage by a protease that cleaves Syn-1 in an FGF:Syn-1 complex, a calcium binding domain and a domain containing a casein kinase II phosphorylation site.

39. A truncated Syn-1 molecule, comprising an amino acid sequence of SEQ ID NO:2 having a deletion such that the truncated Syn-1 molecule lacks at least one of the domains selected from the group consisting of a lipid binding domain, a domain containing a site for cleavage by a protease that cleaves Syn-1 in an FGF:Syn-1 complex, a calcium binding domain and a domain containing a casein kinase II phosphorylation site.

40. A nucleic acid molecule encoding a truncated Syn-1 molecule, comprising a nucleic acid sequence of SEQ ID NO:3 or portion thereof.

41. A truncated Syn-1 molecule, comprising an amino acid sequence of SEQ ID NO:4 or portion thereof.

42. A nucleic acid molecule encoding a truncated Syn-1 molecule, comprising a nucleic acid sequence of SEQ ID NO:5 or portion thereof.

43. A truncated Syn-1 molecule, comprising an amino acid sequence of SEQ ID NO:6 or portion thereof.

44. A nucleic acid molecule encoding a Syn-1 molecule that lacks a site for cleavage by a protease that cleaves Syn-1 in an FGF-1:Syn-1 complex.

45. A method of regulating FGF-1 export from a cell comprising administering a therapeutic agent in an amount sufficient to inhibit the export of FGF-1, wherein said therapeutic agent inhibits the binding of a complex comprising FGF-1 to a cellular membrane, wherein the formation of said complex is a prerequisite to FGF-1 export.

46. The method of claim 45 wherein the cellular membrane is a plasma membrane or a cytoplasmically accessible component thereof.

47. The method of claim 46 wherein the cytoplasmically accessible component comprises a phosphatidylserine molecule.

48. The method of claim 45 wherein the cellular membrane is an intracellular vesicle membrane or a cytoplasmically accessible component thereof.

49. The method of claim 48 wherein the cytoplasmically accessible component comprises a phosphatidylserine molecule.

50. The method of claim 45 wherein said complex further comprises Syn-1.

51. A method of regulating FGF-1 export from a cell comprising administering a therapeutic agent in an amount sufficient to inhibit the export of FGF-1, wherein said therapeutic agent inhibits the release of a complex comprising FGF-1 from a cellular membrane, wherein the formation of said complex is a prerequisite to FGF-1 export;

52. The method of claim 51 wherein the cellular membrane is a plasma membrane or a cytoplasmically accessible component thereof

53. The method of claim 52 wherein the cytoplasmically accessible component comprises a phosphatidylserine molecule.

54. The method of claim 51 wherein the cellular membrane is an intracellular vesicle membrane or a cytoplasmically accessible component thereof.

55. The method of claim 54 wherein the cytoplasmically accessible component comprises a phosphatidylserine molecule.

56. The method of claim 51 wherein said complex further comprises Syn-1 .

57. A method of regulating FGF-1 export from a cell comprising administering a therapeutic agent in an amount sufficient to promote the export of FGF-1, wherein said therapeutic agent promotes the binding of a complex comprising FGF-1 to a cellular membrane, wherein the formation of said complex is a prerequisite to FGF-1 export.

58. The method of claim 57 wherein the cellular membrane is a plasma membrane or a cytoplasmically accessible component thereof.

59. The method of claim 58 wherein the cytoplasmically accessible component comprises a phosphatidylserine molecule.

60. The method of claim 57 wherein the cellular membrane is an intracellular vesicle or a cytoplasmically accessible component thereof.

61. The method of claim 60 wherein the cytoplasmically accessible component comprises a phosphatidylserine molecule.

62. The method of claim 57 wherein said complex further comprises Syn-1.

63. A method of regulating FGF-1 export from a cell comprising administering a therapeutic agent in an amount sufficient to promote the export of FGF-1, wherein said therapeutic agent promotes the release of a complex comprising FGF-1 from a cellular membrane, wherein the formation of said complex is a prerequisite to FGF-1 export.

64. The method of claim 63 wherein the cellular membrane is a plasma membrane or a cytoplasmically accessible component thereof.

65. The method of claim 64 wherein the cytoplasmically accessible component comprises a phosphatidylserine molecule.

66. The method of claim 63 wherein the cellular membrane is an intracellular vesicle or a cytoplasmically accessible component thereof.

67. The method of claim 66 wherein the cytoplasmically accessible component comprises a phosphatidylserine molecule.

68. The method of claim 63 wherein said complex further comprises Syn-1.

69. The method of any one of claims 1, 8, 45, 51, 57 and 63 wherein said complex further comprises S100A13.

70. A method according to claim 69 wherein said therapeutic agent binds to S100A13.

71. The method of claim 70 wherein the therapeutic agent is an amlexanox compound.

72. The method of claim 71 wherein the amlexanox compound is selected from the group consisting of amlexanox AA673, amlexanox derivative AA617 and amlexanox derivative AA648.

73. A method of identifying a component in a complex the formation of which is a prerequisite to FGF-1 export, comprising:
isolating from a cell capable of expressing and exporting FGF-1 a complex comprising FGF-1; and determining the presence in said isolated complex of one or more molecular species, and therefrom identifying a component of said complex.

74. A method of identifying a component in a complex, the formation of which is a prerequisite to FGF-1 export, comprising:
a. isolating from a cell capable of expressing and exporting FGF-1 a complex comprising FGF-1;
b. determining whether formation of said isolated complex is a prerequisite to FGF-1 export; and
c. detecting the presence in isolated complexes from step (b) of one or more molecular species, and therefrom identifying a component of said complex.

75. A method of identifying a component in a complex the formation of which is a prerequisite to FGF-1 export, comprising:
a. isolating. from a cell capable of expressing and exporting FGF-1 a complex comprising FGF-1 and Syn-1; and
b. detecting the presence in said isolated complex of one or more molecular species, and therefrom identifying a component of said complex.

76. The method of claim 75 wherein Syn-1 is a truncated Syn-1.

77. The method of any one of claims 73, 74, 75 or 76 wherein the molecular species preferentially associates with FGF-1.

78. The method of any one of claims 73, 74, 75 or 76 wherein the molecular species associates indirectly with FGF-1.

79. The method of claim 73 wherein isolation of the complex comprising FGF-1 follows export of said complex.

80. The method of claim 73 wherein isolation of the complex comprising FGF-1 precedes export of said complex.

81. The method of claim 73 wherein the complex comprising FGF-1 further comprises dimeric FGF-1.

82. The method of claim 73 wherein the complex comprising FGF-1 is isolated from a source selected from the group consisting of cell conditioned medium, cultured cells and a biological tissue.

83. The method of claim 82 wherein the biological tissue is brain.

84. The method of claim 73 wherein the complex comprising FGF-1 further comprises Syn-1.

85. The method of claim 73 wherein the complex comprising FGF-1 further comprises S100A13.

86. The method of claim 73 wherein FGF-1 in an isolated complex is a product of an endogenous gene.

87. The method of claim 73 wherein FGF-1 in an isolated complex is a product of a transfected gene.

88. The method of claim 87 wherein the transfected gene encodes full length FGF-1.

89. The method of claim 87 wherein the transfected gene encodes truncated FGF-1.

90. The method of claim 89 wherein the transfected gene encodes FGF-1(₂₁₋₁₅₄).

91. The method of claim 89 wherein the transfected gene encodes FGF-1 having an N-terminal deletion of at least 14 amino acids.

92. The method of claim 73 wherein the complex comprising FGF-1 is isolated by an affinity technique.

93. The method of claim 92 wherein the affinity technique is an immunological technique.

94. The method of claim 93 wherein the immunological technique is selected from the group consisting of immunoaffinity chromatography, immunoprecipitation, and solid phase immunoadsorption.

95. The method of claim 92 wherein the affinity technique is heparin binding.

96. A method of identifying an agent that inhibits formation of a complex comprising FGF-1 wherein the formation of said complex is a prerequisite to FGF-1 export, comprising:
exposing to a candidate agent a cell capable of expressing FGF-1 and of forming a complex which is a prerequisite to exporting FGF-1, said complex including FGF-1; and
determining the presence or absence of said complex, and therefrom identifying an agent that inhibits the formation of said complex that is a prerequisite to FGF-1 export.

97. A method of identifying an agent that promotes formation of a complex comprising FGF-1 wherein the formation of said complex is a prerequisite to FGF-1 export, comprising:
exposing to a candidate agent a cell capable of expressing FGF-1 and of forming a complex which is a prerequisite to expressing and exporting FGF-1, said complex including FGF-1; and
determining the presence or absence of said complex, and therefrom identifying an agent that promotes the formation of said complex that is a prerequisite to FGF-1 export.

98. The method of either claim 96 or claim 97 wherein the step of determining the presence or absence of the complex comprises determining the presence or absence of at least one molecular species that preferentially associates with FGF-1.

99. The method of either claim 96 or claim 97 wherein the step of determining the presence or absence of the complex comprises determining the presence or absence of at least one molecular species that associates indirectly with FGF-1.

100. A nucleic acid molecule encoding a truncated FGF-1, comprising a nucleotide sequence of SEQ ID NO:7 having a deletion.

101. The nucleic acid molecule of claim 97 wherein the truncated FGF-1 binds Syn-1.

102. The nucleic acid molecule of claim 100 wherein the truncated FGF-1 binds phosphatidylserine.

103. A truncated FGF-1 molecule, comprising an amino acid sequence of SEQ ID NO:8 having a deletion.

104. The truncated FGF-1 molecule of claim 103 wherein the truncated FGF-1 binds Syn-1.

105. The truncated FGF-1 molecule of claim 103 wherein the truncated FGF-1 binds phosphatidylserine.

106. A nucleic acid molecule encoding a fusion protein comprising a nucleic acid sequence encoding a truncated FGF-1 that binds Syn-1 and a nucleic acid sequence encoding a desired polypeptide.

107. A nucleic acid molecule encoding a fusion protein comprising a nucleic acid sequence encoding a truncated FGF-1 that binds phosphatidylserine and a nucleic acid sequence encoding a desired polypeptide.


SEQUENCE LISTING (10 sequences)

SEQ ID NO: 1-8 - skipped (8 entries)

SEQ ID NO: 9 - DNA, 20 bp, Artificial Sequence
  Sense PCR Primer
ccattgccaccgtgggcctt

SEQ ID NO: 10 - DNA, 20 bp, Artificial Sequence
  Antisense PCR Primer
tccaaaacagttaccaccac